(19) United States
(12) Patent Application Publication
(10) Pub. No.: US 2024/0374617 A1
(43) Pub. Date: Nov. 14, 2024

(54) METHODS OF ACTIVATING MICROGLIAL CELLS

(71) Applicants: The Research Foundation for the State University of New York, Albany, NY (US); SYRACUSE UNIVERSITY, SYRACUSE, NY (US)

(72) Inventors: William Kerr, Syracuse, NY (US); Chiara Pedicone, Syracuse, NY (US); John Chisolm, Syracuse, NY (US); Shawn Dormann, Syracuse, NY (US)

(21) Appl. No.: 18/783,183

(22) Filed: Jul. 24, 2024

Related U.S. Application Data
(63) Continuation of application No. 17/262,784, filed on Jan. 25, 2021, now abandoned.
(60) Provisional application No. 62/875,831, filed on Jul. 18, 2019.
(60) Provisional application No. 62/712,566, filed on Jul. 31, 2018.

(51) Int. Cl. A61K 31/575; A61K 31/4045; A61K 31/568; A61K 31/5685; A61K 31/57

(52) U.S. Cl. CPC A61K 31/575; A61K 31/4045; A61K 31/568; A61K 31/5685; A61K 31/57

(57) ABSTRACT

The present disclosure provides methods of using compositions that inhibit SII2-containing inositol 5′-phosphatases (SHIPs) for activating microglial cells, as well as methods for using such compositions for treatment or ameliorating of neurodegenerative disorders in a subject.

### RELATED APPLICATIONS

This application claims priority from U.S. Provisional Patent Application No. 62/712,566, filed on Jul. 31, 2018; and U.S. Provisional Patent Application No. 62/875,831, filed Jul. 18, 2019, both of which are incorporated by reference herein in their entireties.

### FIELD OF THE INVENTION

The disclosure relates generally to the biological effect of SHIP1/2 inhibitors on microglial cells.

### BACKGROUND

Genome wide association studies (GWAS) have identified single nucleotide polymorphisms (SNP)s in the INPP5D (SHIP1) gene that are associated with Alzheimer disease (AD) risk. A recent study showed that such SNPs are associated with increased SHIP1 mRNA expression suggesting that targeting SHIP1 activity in vivo could be a potential therapeutic strategy in AD. Other studies also suggest that dampening or reducing SHIP1 activity could have potential efficacy in AD. For example, SHIP1 limits signaling by key receptors (TREM2, CCR2, Dectin1) that promote microglial survival and proliferation in the CNS (TREM2, Dectin1), their effector function including responding to Aβ plaques (TREM2) or the migration of peripheral monocyte-macrophages (mo-MΦ) into the CNS and toward Aβ plaques (CCR2). In the peripheral immune system, SHIP1 and its paralog SHIP2 are co-expressed, including in terminally differentiated macrophages. SHIP1 influences signaling downstream of the following receptors elaborated by terminally differentiated myeloid cells: TREM2, Dectin1, FcγR, c-Kit, G-CSF-R and CCR2 while SHIP2 regulates M-CSF and FcγR signaling. Thus, SHIP1 and/or SHIP2 might also play a role in cell signaling pathways that control microglial proliferation, survival, effector functions and/or their chemotaxis.

### SUMMARY

The summary of the technology described herein is non-limiting and other features and advantages of the invention will be apparent from the following detailed description of the technology, and from the claims.

It is demonstrated that pan-SHIP1/2 inhibitory compounds (e.g., K149, K118, K161) can increase growth of microglial cells, increase the size of the lysosomal/phagosomal compartment in microglial cells, and increase the phagocytosis of the beta-amyloid (1-42) peptide.

The instant disclosure provides a method for activating microglial cells with inhibitors of SHIP. Accordingly, in one aspect, the instant disclosure provides a method of activating microglial cells in a subject suffering from an illness or condition for which microglial cells provide a host defense, said method comprising: administering a safe and effective amount of a SHIP1 inhibitor, SHIP2, and/or a pan-SHIP1/2 inhibitor to the subject. In some embodiments, the SHIP inhibitor is a pan-SHIP1/2 inhibitor.

In some embodiments, the illness or condition is a neurodegenerative disorder. In some embodiments, the neurodegenerative disorder is characterized by death of neurons. In some embodiments, the neurodegenerative disorder is characterized by misfolded proteins. In some embodiments, the misfolded proteins are beta-amyloid, comprising the beta-amyloid (1-42) peptide.

In some embodiments, the illness or condition is Alzheimer's disease.

In some embodiments, the activating microglia results in one or more of the following: promotion of the growth of microglial cells, an increase in the size of the lysosomal/phagosomal compartment in microglial cells, an increase in the phagocytosis of dead neurons by microglial cells, and an increase in the phagocytosis of beta-amyloid protein or beta-amyloid (1-42) peptide by microglial cells.

In some embodiments, activating microglial cells results in an increase in the size of the lysosomal/phagosomal compartment in microglial cells. In some embodiments, activating microglial cells results in an increase in the phagocytosis of dead neurons by microglial cells. In some embodiments, activating microglial cells results in an increase in the phagocytosis of beta-amyloid protein or beta-amyloid (1-42) peptide by microglial cells. In some embodiments, the microglial cells showing an increase in the phagocytosis of dead neurons are CD11b+CD45 low cells. In some embodiments, the microglial cells showing an increase in the phagocytosis of beta amyloid protein or beta-amyloid peptide are CD11b+CD45 low cells.

In some embodiments, the SHIP1 inhibitor, SHIP2 inhibitor and/or a pan-SHIP1/2 inhibitor for activating microglial cells is a SHIP inhibitor compound of formula (I):

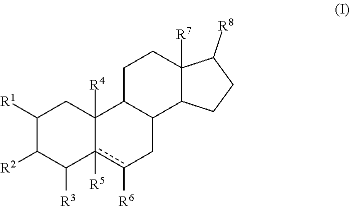

wherein
[symbol] represents a single or double bond;
R¹ is selected from the group consisting of hydrogen, hydroxy, mercapto, C₁-C₆ alkoxy, aryloxy, C₁-C₆ alkylthio, C₆-C₁₂ arylthio, C₁-C₆ alkylcarbonamido, C₆-C₁₂ arylcarbonamido, C₁-C₆ alkylsulfonamido, C₆-C₁₂ arylsulfonamido, substituted or unsubstituted amino, oxycarbonyl and C₁-C₆ aminoalkyl;
R² is selected from the group consisting of hydrogen, hydroxy, mercapto, C₁-C₆ alkoxy, aryloxy, C₁-C₆ alkylthio, C₆-C₁₂ arylthio, C₁-C₆ alkylcarbonamido, C₆-C₁₂ arylcarbonamido, C₁-C₆ alkylsulfonamido, C₆-C₁₂ arylsulfonamido, substituted or unsubstituted amino, oxycarbonyl and C₁-C₆ aminoalkyl; R³ is hydrogen, C₁-C₄ alkyl, or C₁-C₄ haloalkyl;
R³ is hydrogen, C₁-C₄ alkyl, or C₁-C₄ haloalkyl;
R⁴ is hydrogen, C₁-C₄ alkyl, or C₁-C₄ haloalkyl;
R⁵ is hydrogen, C₁-C₄ alkyl, hydroxy, C₁-C₆ alkoxy, mercapto, C₁-C₆ alkylthio, substituted or unsubstituted amino, or C₁-C₄ haloalkyl;
R¹ is hydrogen, C₁-C₄ alkyl, hydroxy, C₁-C₆ alkoxy, mercapto, C₁-C₆ alkylthio, substituted or unsubstituted amino, or C₁-C₄ haloalkyl;
R⁷ is hydrogen, C₁-C₄ alkyl, or C₁-C₄ haloalkyl; and
R⁸ is hydrogen, C₁-C₁₂ alkyl, or C₁-C₁₂ haloalkyl, and pharmaceutically acceptable esters, salts, and prodrugs thereof. In some embodiments, the SHIP inhibitor is a pan-SHIP1/2 inhibitor.

In some embodiments, R¹ and R² are a substituted or unsubstituted amino.

In some embodiments, R¹ and R² are NH₂ or NH₃Cl.

In some embodiments, the compound of Formula (I) is selected from the group consisting of:

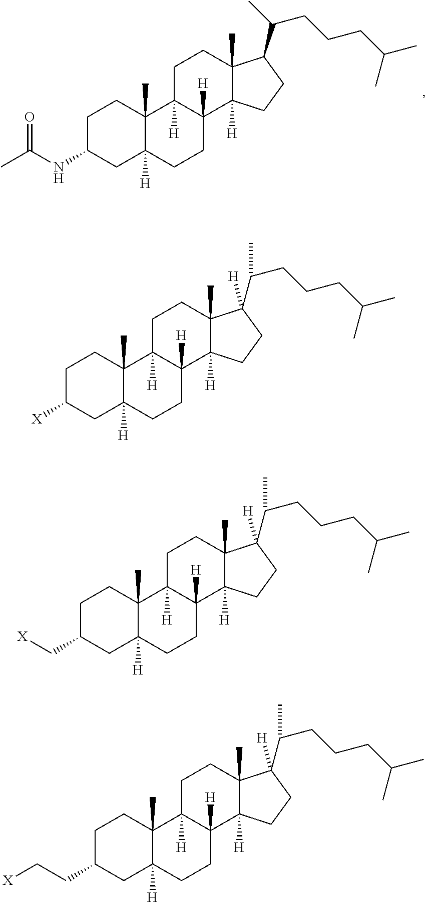

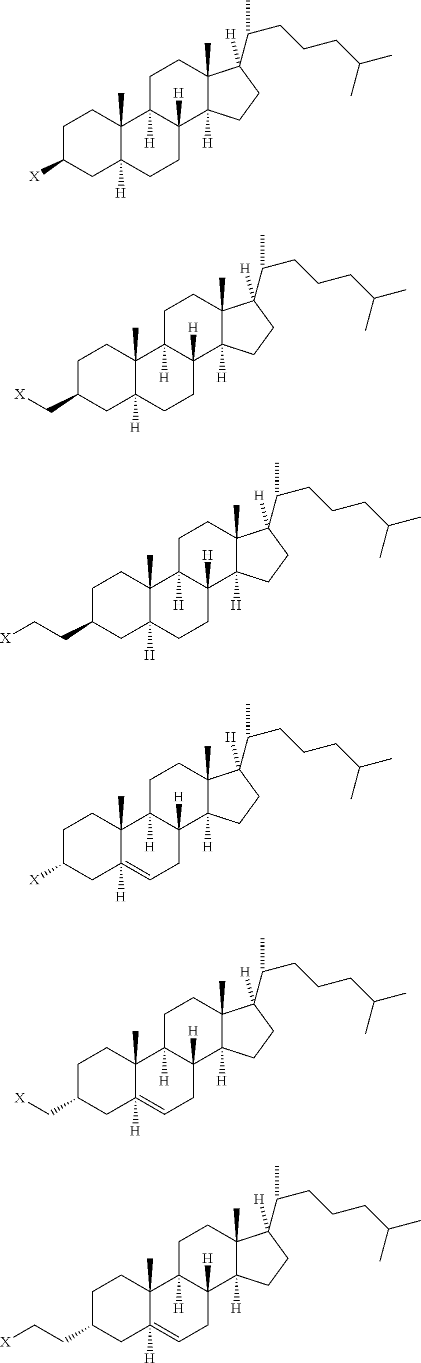

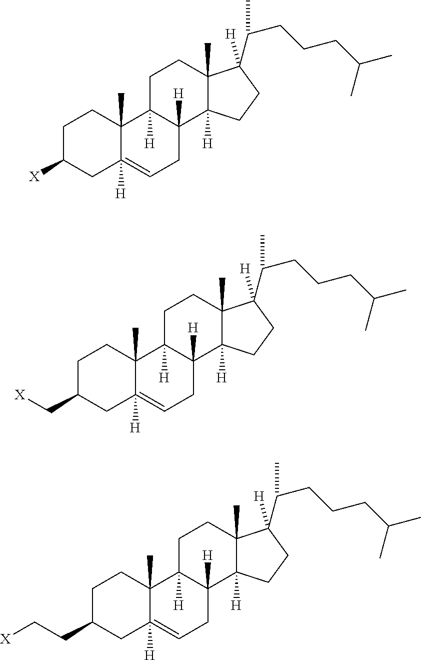

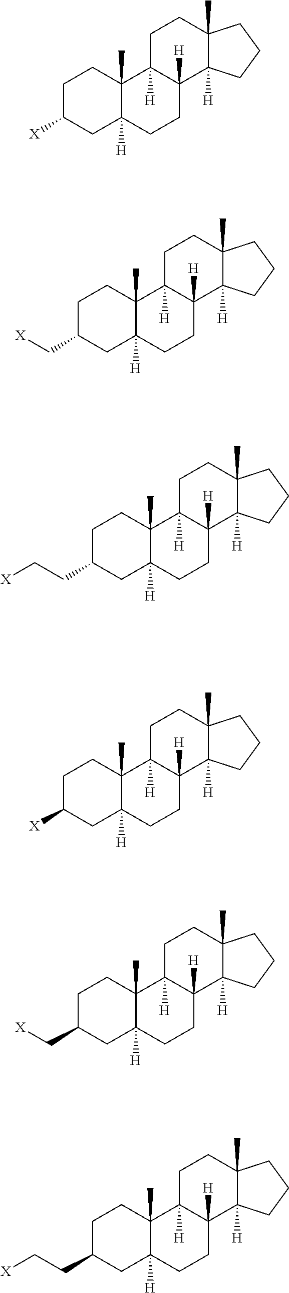

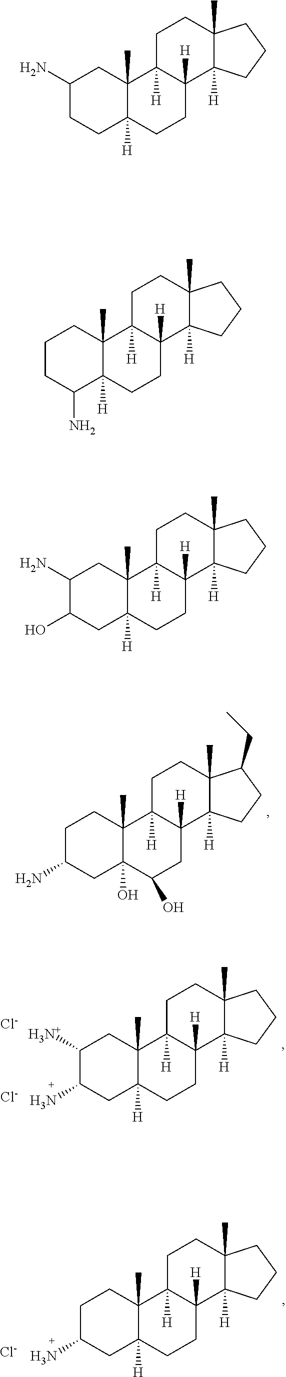

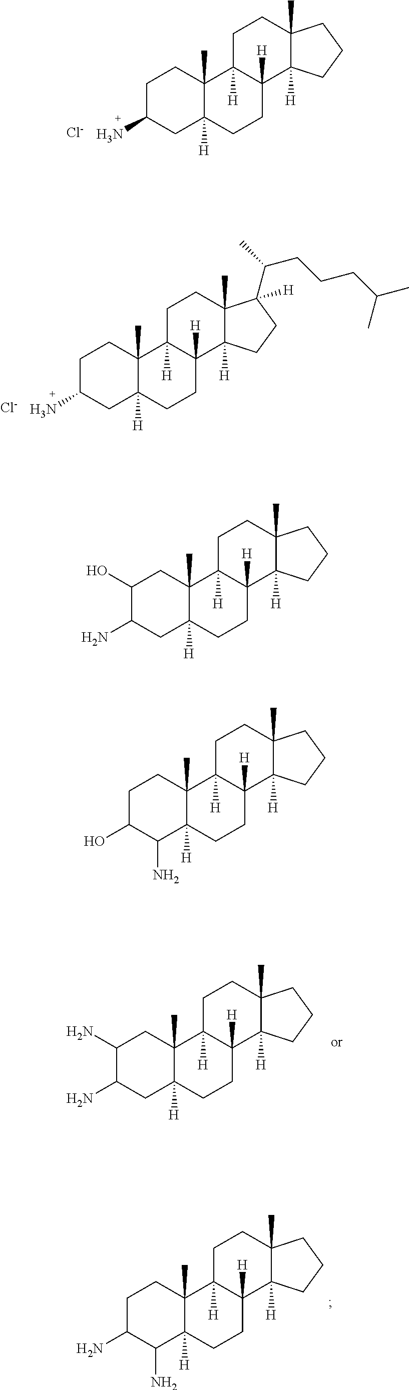

and
pharmaceutically acceptable salts thereof, wherein X=NR₂, NRCOR, NHCONR₂, OR, SR, OCOR, OCONR₂, or NHCNHNH₂, and wherein R=H, alkyl, cycloalkyl, aryl, or benzyl.

In some embodiments, the compound of Formula (I) is a compound of Formula (II):

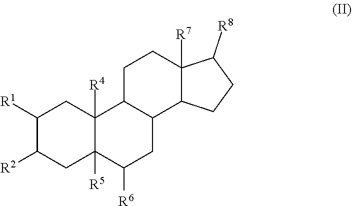

wherein
R¹ is selected from the group consisting of hydrogen, hydroxy, substituted or unsubstituted amino, and C₁-C₆ aminoalkyl;
R² is selected from the group consisting of hydrogen, hydroxy, substituted or unsubstituted amino, and C₁-C₆ aminoalkyl;
R⁴ is C₁-C₄ alkyl;
R⁵ is hydrogen, C₁-C₄ alkyl, or hydroxy;
R⁶ is hydrogen, C₁-C₄ alkyl, or hydroxy;
R⁷ is C₁-C₄ alkyl; and
R⁸ is hydrogen or C₁-C₁₂ alkyl, and pharmaceutically acceptable esters, salts, and prodrugs thereof.

In some embodiments, the substituted or unsubstituted amino is NH₂ or NH₃Cl.

In some embodiments, R¹ and R² are a substituted or unsubstituted amino.

In some embodiments, R¹ and R² are NH₂ or NH₃Cl.

In some embodiments, R⁵ and R⁶ are hydroxy.

In some embodiments, R⁸ s C₁-C₁₂ alkyl.

In some embodiments, R⁸ is hydrogen.

In yet another embodiment, the the compound of Formula (II) is a hydrochloride salt.

In some embodiments, the compound of Formula (II) is selected from the group consisting of:

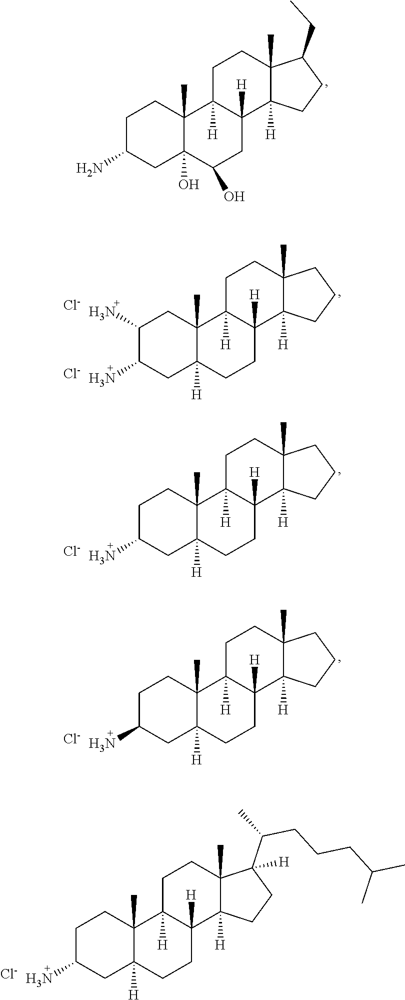

In some embodiments, the pan-SHIP1/2 inhibitor is a compound of Formula (III):

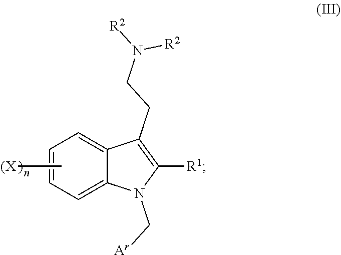

wherein,
Ar is a C₅₋₆ aryl;
R¹ is selected from the group consisting of H, C₁₋₄ alkyl, and C₅₋₆ aryl;
each R² is independently selected from the group consisting of H, C₁₋₄ alkyl, and C₅₋₆ aryl;
each X, if present, is independently selected from the group consisting of halo, C₁₋₄ alkyl, C₅₋₆ aryl, and —Y—R³;
   wherein Y is selected from the group consisting of —S—, —NH—, —O—; and
   R³ is H or C₁₋₄ alkyl; and
n is 0-4, and pharmaceutically acceptable esters, salts, and prodrugs thereof.

In some embodiments, the compound of Formula (III) is selected from the group of:

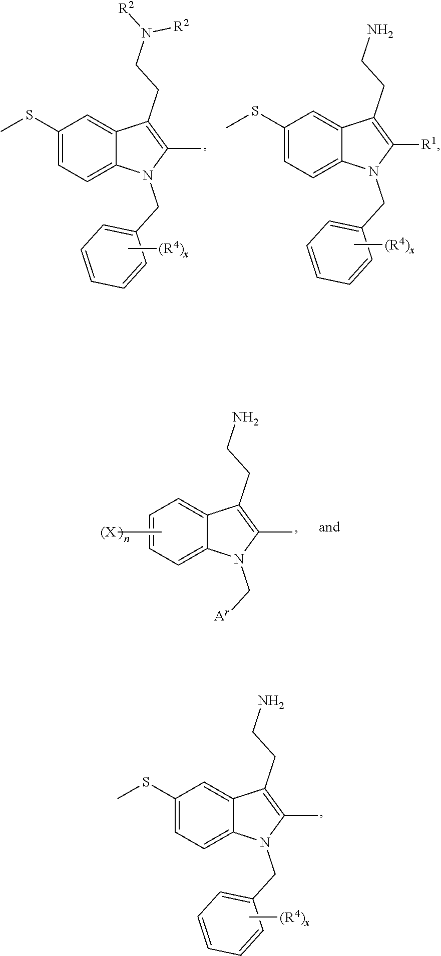

where R⁴ is halo, and x is 0-5, and pharmaceutically acceptable esters, salts, and prodrugs thereof.

In some embodiments, the compound of Formula (III) is selected from the group consisting of:

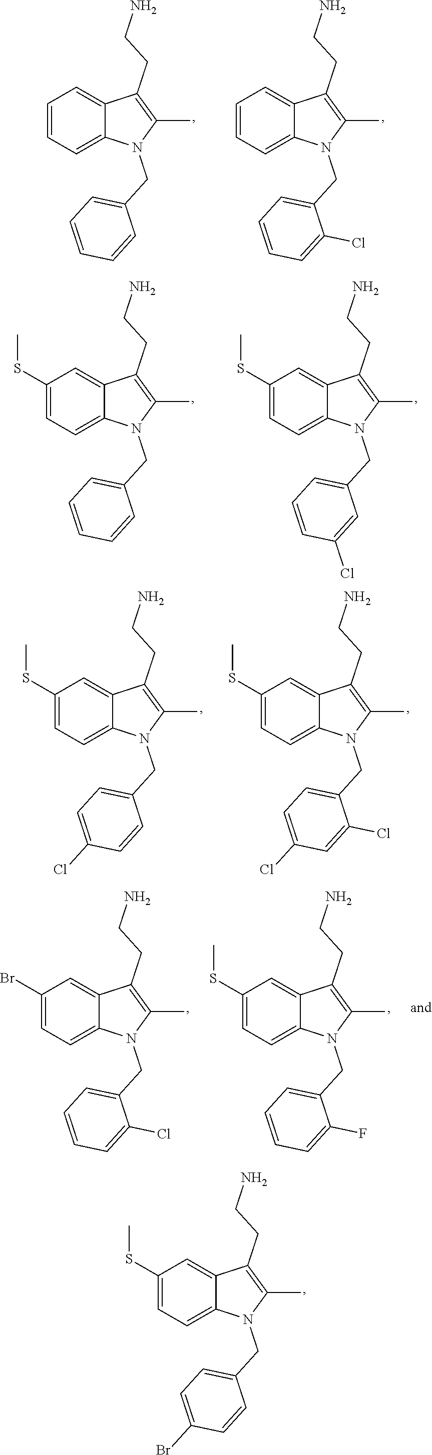

and pharmaceutically acceptable esters, salts, and prodrugs thereof.

The instant disclosure also provides a method of improving one or more symptoms of a neurodegenerative disorder in a subject in need thereof comprising: administering a safe and effective amount of a SHIP1 inhibitor, a SHIP2, and/or a pan-SHIP1/2 inhibitor to the subject, wherein said inhibitor activates microglial cells. In some embodiments, the SHIP inhibitor is a pan-SHIP1/2 inhibitor.

In some embodiments, the subject suffers from a neurodegenerative disorder. In some embodiments, the subject suffers from a neurodegenerative disorder that is characterized by death of neurons. In some embodiments, the subject suffers from a neurodegenerative disorder is characterized by misfolded proteins. In some embodiments, the misfolded proteins are beta-amyloid, comprising the beta-amyloid (1-42) peptide.

In some embodiments, the subject suffers from Alzheimer's disease. In some embodiments, activating microglial cells in a subject results in one or more of the following: promotion of the growth of microglial cells; an increase in the size of the lysosomal/phagosomal compartment in microglial cells; an increase in the phagocytosis of dead neurons by microglial cells; and an increase in the phagocytosis of beta-amyloid protein or beta-amyloid (1-42) peptide by microglial cells.

In some embodiments, activating microglial cells in a subject results in an increase in the size of the lysosomal/phagosomal compartment in microglial cells. In some embodiments, activating microglial cells in a subject results in an increase in the phagocytosis of dead neurons. In some embodiments, activating microglial cells in a subject causes an increase in the phagocytosis of beta-amyloid protein or beta-amyloid (1-42) peptide. In some embodiments, the microglial cells in a subject showing an increase in the phagocytosis of dead neurons are CD11b+CD45 low cells. In some embodiments, the microglial cells in a subject showing an increase in the phagocytosis of beta amyloid protein or beta-amyloid peptide are CD11b+CD45 low cells.

In some embodiments, the SHIP1 inhibitor, SHIP2, and/or pan-SHIP1/2 inhibitor is administered to a subject in a continuous manner or in a pulsatile manner. In some embodiments, the SHIP1 inhibitor, SHIP2, and/or pan-SHIP1/2 inhibitor is administered to the subject at least daily for a period of at least 2, 3, 4, 5, 6, 7, 8, 9, 10, 12, 14, 16, 18, 20 or more days. In some embodiments, the SHIP1 inhibitor, SHIP2 inhibitor, and/or pan-SHIP1/2 inhibitor is administered peripherally to the subject.

In some embodiments, the pan-SHIP1/2 inhibitor is a compound of formula (I):

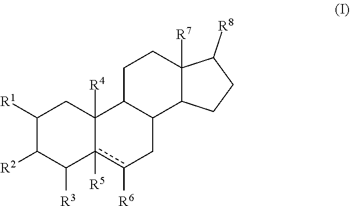

wherein
[symbol] represents a single or double bond;
R¹ is selected from the group consisting of hydrogen, hydroxy, mercapto, C₁-C₆ alkoxy, aryloxy, C₁-C₆ alkylthio, C₆-C₁₂ arylthio, C₁-C₆ alkylcarbonamido, C₆-C₁₂ arylcarbonamido, C₁-C₆ alkylsulfonamido, C₆-C₁₂ arylsulfonamido, substituted or unsubstituted amino, oxycarbonyl and C₁-C₆ aminoalkyl;
R² is selected from the group consisting of hydrogen, hydroxy, mercapto, C₁-C₆ alkoxy, aryloxy, C₁-C₆ alkylthio, C₆-C₁₂ arylthio, C₁-C₆ alkylcarbonamido, C₆-C₁₂ arylcarbonamido, C₁-C₆ alkylsulfonamido, C₆-C₁₂ arylsulfonamido, substituted or unsubstituted amino, oxycarbonyl and C₁-C₆ aminoalkyl; R³ is hydrogen, C₁-C₄ alkyl, or C₁-C₄ haloalkyl;
R³ is hydrogen, C₁-C₄ alkyl, or C₁-C₄ haloalkyl;
R⁴ is hydrogen, C₁-C₄ alkyl, or C₁-C₄ haloalkyl;
R⁵ is hydrogen, C₁-C₄ alkyl, hydroxy, C₁-C₆ alkoxy, mercapto, C₁-C₆ alkylthio, substituted or unsubstituted amino, or C₁-C₄ haloalkyl;
R⁶ is hydrogen, C₁-C₄ alkyl, hydroxy, C₁-C₆ alkoxy, mercapto, C₁-C₆ alkylthio, substituted or unsubstituted amino, or C₁-C₄ haloalkyl;
R⁷ is hydrogen, C₁-C₄ alkyl, or C₁-C₄ haloalkyl; and
R⁸ is hydrogen, C₁-C₁₂ alkyl, or C₁-C₁₂ haloalkyl, and pharmaceutically acceptable esters, salts, and prodrugs thereof.

In some embodiments, R¹ and R² are a substituted or unsubstituted amino.

In some embodiments, R¹ and R² are NH₂ or NH₃Cl.

In some embodiments, the compound of Formula (I) is selected from the group consisting of:

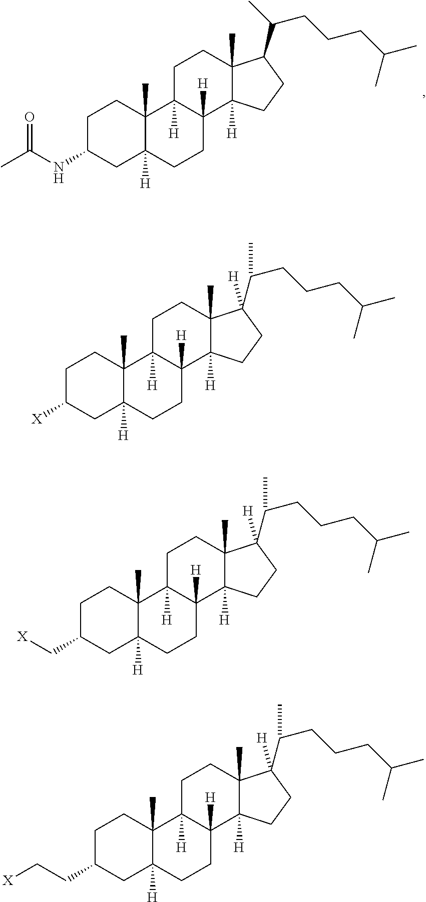

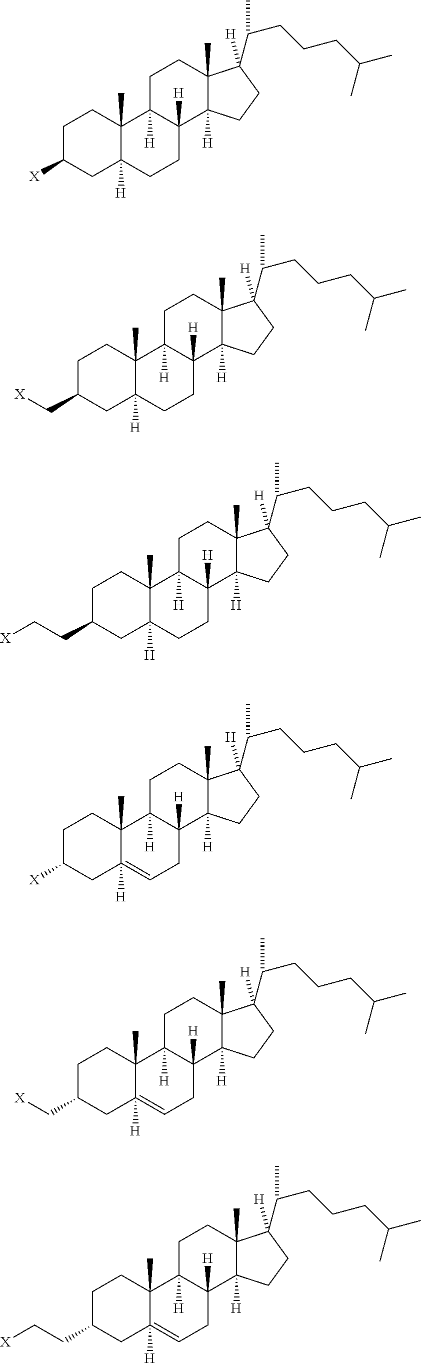

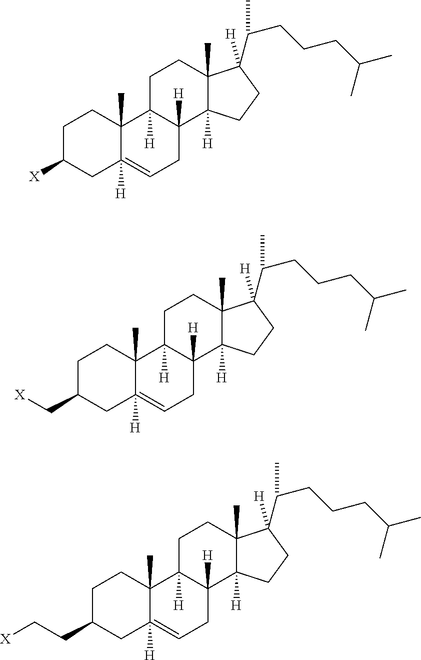

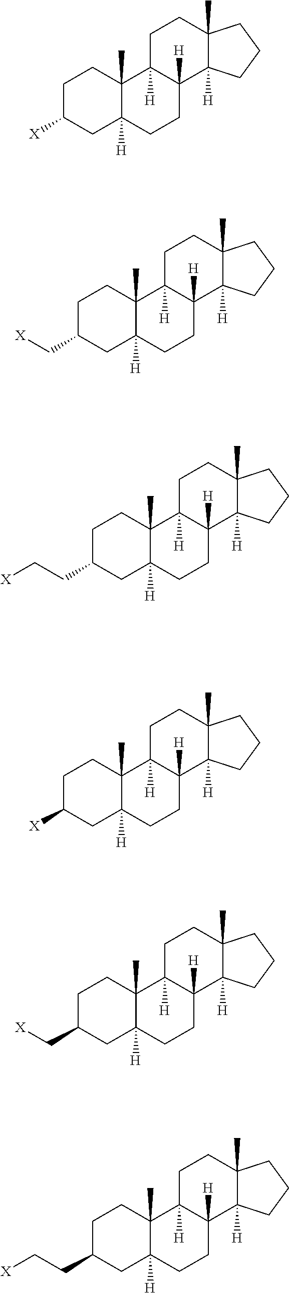

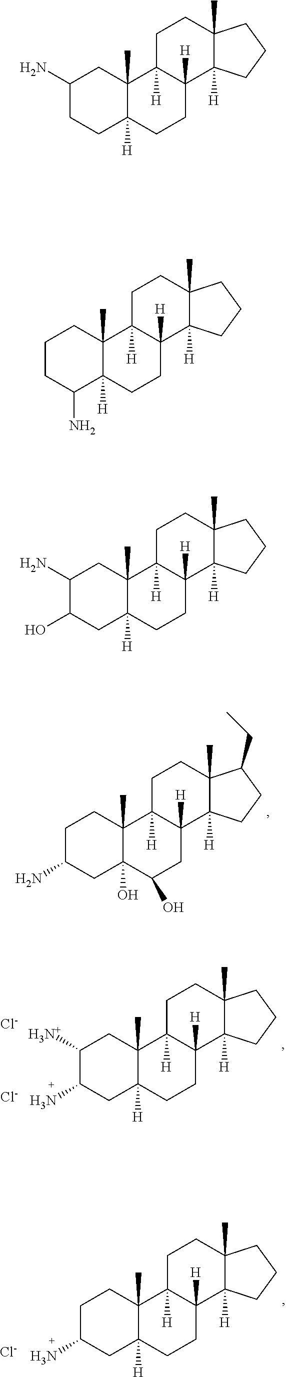

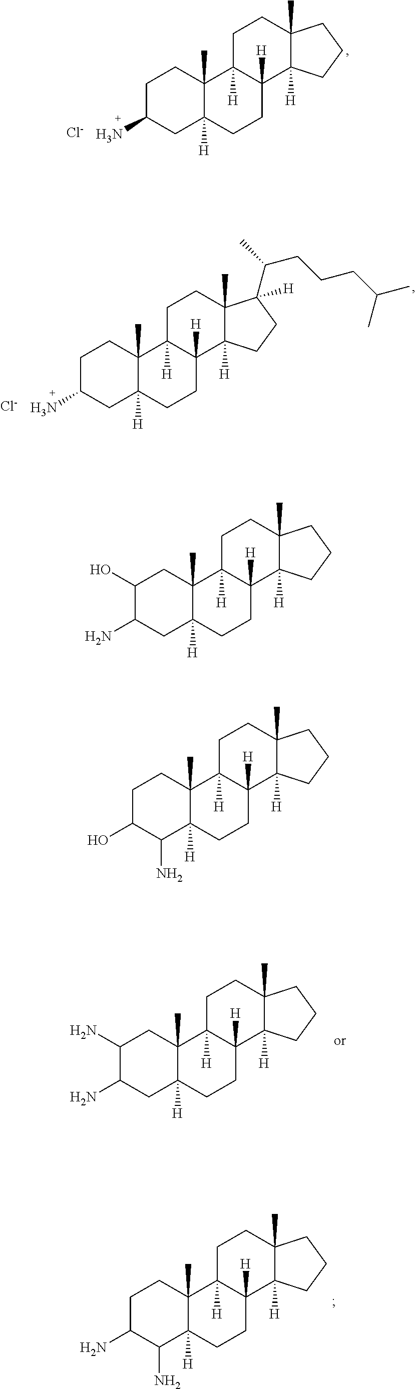

and
pharmaceutically acceptable salts thereof, wherein X=NR₂, NRCOR, NHCONR₂, OR, SR, OCOR, OCONR₂, or NHCNHNH₂, and wherein R=H, alkyl, cycloalkyl, aryl, or benzyl.

In some embodiments, the pan-SHIP1/2 inhibitor for administering to a subject is a compound of Formula (II):

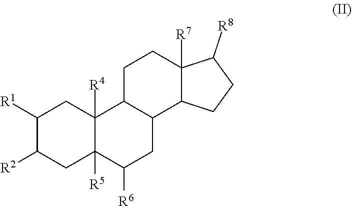

wherein
R¹ is selected from the group consisting of hydrogen, hydroxy, substituted or unsubstituted amino, and C₁-C₆ aminoalkyl;
R² is selected from the group consisting of hydrogen, hydroxy, substituted or unsubstituted amino, and C₁-C₆ aminoalkyl;
R⁴ is C₁-C₄ alkyl;
R⁵ is hydrogen, C₁-C₄ alkyl, or hydroxy;
R⁶ is hydrogen, C₁-C₄ alkyl, or hydroxy;
R⁷ is C₁-C₄ alkyl; and
R⁸ is hydrogen or C₁-C₁₂ alkyl, and pharmaceutically acceptable esters, salts, and prodrugs thereof.

In some embodiments, the substituted or unsubstituted amino is NH₂ or NH₃Cl.

In some embodiments, R¹ and R² are a substituted or unsubstituted amino.

In some embodiments, R¹ and R² are NH₂ or NH₃Cl.

In some embodiments, R⁵ and R⁶ are hydroxy.

In some embodiments, R⁸ is C₁-C₁₂ alkyl.

In some embodiments, R⁸ is hydrogen.

In yet another embodiment, the the compound of Formula (II) is a hydrochloride salt.

In some embodiments, the compound of Formula (II) is selected from the group consisting of:

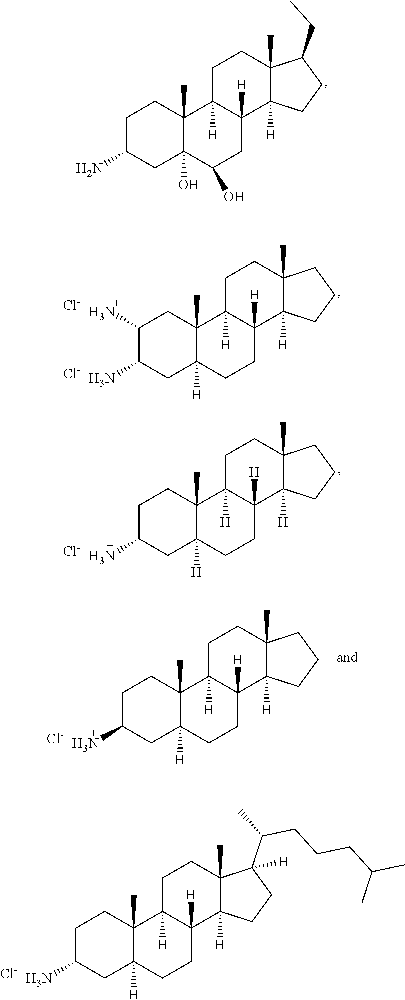

In some embodiments, the pan-SHIP1/2 inhibitor for administering to a subject is a SHIP inhibitor compound of Formula (III):

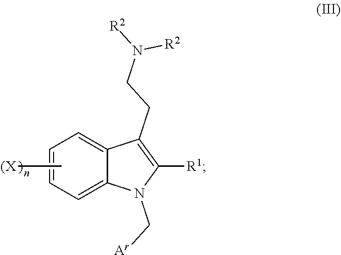

wherein,
Ar is a C₅₋₆ aryl;
R¹ is selected from the group consisting of H, C₁₋₄ alkyl, and C₅₋₆ aryl;
each R² is independently selected from the group consisting of H, C₁₋₄ alkyl, and C₅₋₆ aryl;
each X, if present, is independently selected from the group consisting of halo, C₁₋₄ alkyl, C₅₋₆ aryl, and —Y—R³;
   wherein Y is selected from the group consisting of —S—, —NH—, —O—; and
   R³ is H or C₁₋₄ alkyl; and
n is 0-4, and pharmaceutically acceptable esters, salts, and prodrugs thereof.

In some embodiments, the compound of Formula (III) is selected from the group of:

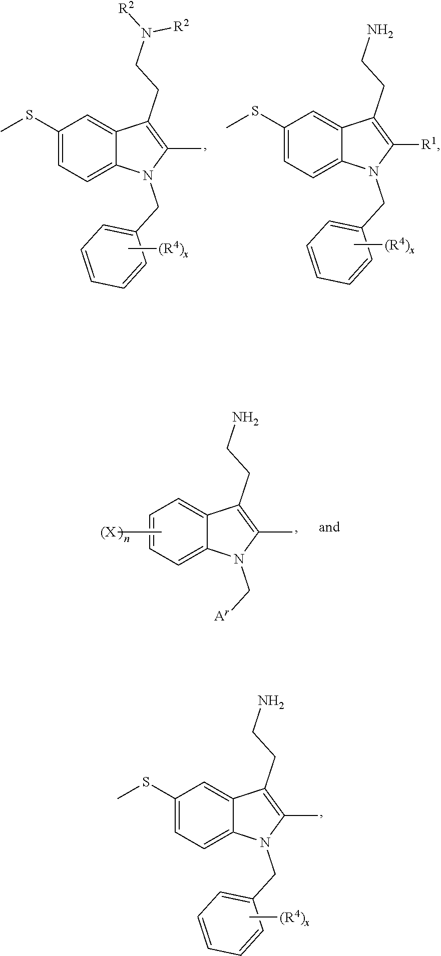

where R⁴ is halo, and x is 0-5, and pharmaceutically acceptable esters, salts, and prodrugs thereof.

In some embodiments, the compound of Formula (III) is selected from the group consisting of:

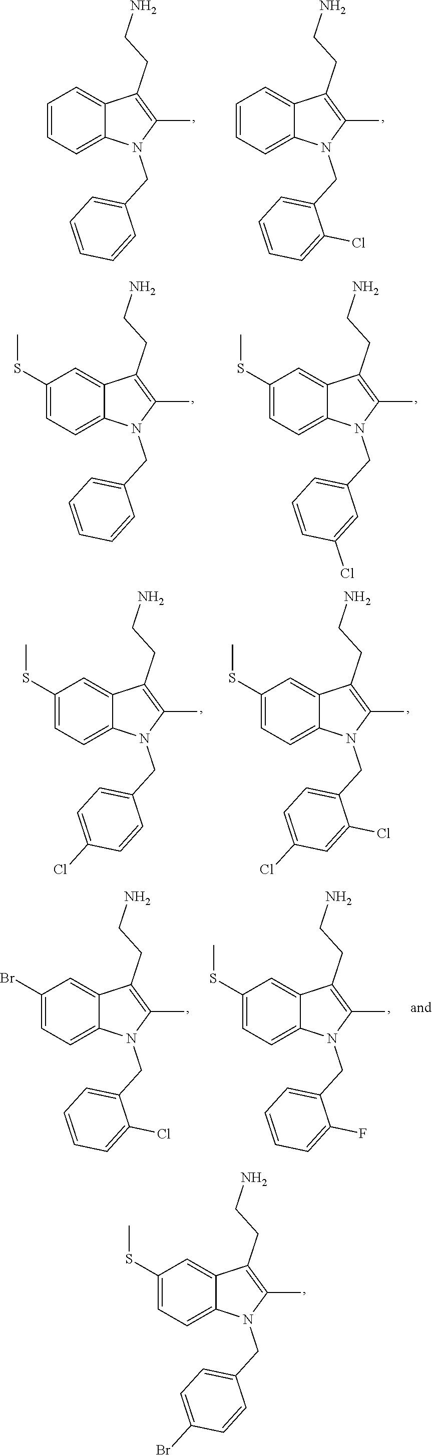

and pharmaceutically acceptable esters, salts, and prodrugs thereof.

In some embodiments, the compounds disclosed herein are administered to a subject by any suitable route, such as oral or parenteral administration. In some embodiments, the parenteral administration is intravenous administration.

### BRIEF DESCRIPTION OF THE DRAWINGS

FIG. 1 is a drawing of the Src homology 2 (SH2) domain containing inositol polyphosphate 5-phosphatase enzyme and its effects on phosphoinositol intermediates.

FIG. 2 shows the structures of the SHIP inhibitors 3AC, K118, K149, AS19490, K116, K161, and K185.

FIG. 3 shows quantitation results of the expression of SHIP1 and SHIP2 in microglia cell lines assessed by flow cytometry (A) and primary microglia and microglial cell lines by Western Blotting (B)

FIG. 4 shows the proliferation of BV2 (FIG. 4A) and SIM-A9 (FIG. 4B) cells in response to SHIP inhibitors 3AC (circles), K118 (squares) and K149 (triangles). The experiment is representative of three independent experiments, analyzed with a two way ANOVA (*p<0.05, **p<0.01, ***p<0.001, ****p<0.0001).

FIG. 5 provides histograms of lysotracker staining in BV2 cells incubated with 3AC (FIG. 5A), K118 (FIG. 5B) and K149 (FIG. 5C). K118 effects on BV2 cells and primary microglial cells are compared in FIG. 5D-G. Quantitation of lysotracker phagocytosis in the absence and presence of the indicated SHIP1, SHIP2, and pan-SHIP inhibitors are shown in FIG. 5H and FIG. 5I, and effects of K161 on primary microglial cells in FIG. 5J. Brown-Forsythe and Welch ANOVA (*p<0.05, **p<0.01, ***p<0.001, ****p<0.0001) were used. Confocal microscopic images are shown in FIG. 5K, and quantitation of the results obtained with inhibitor K161 are shown in FIG. 5L.

FIG. 6 shows effects of SHIP1/2 inhibition on the phagocytosis of dead neurons by Sim-A9 (FIG. 6A), BV2 cells (FIG. 6B and FIG. 6C), and primary microglial cells (FIG. 6D). The confocal images further visualize the phagocytosis of dead neurons by BV2 cells in FIG. 6E (left images) and by primary microglial cells in FIG. 6F (right images), following stimulation with water (control; top) or K161 (bottom). Brown-Forsythe and Welch ANOVA. or t test with Welch correction (*p<0.05, **p<0.01, ***p<0.001, ****p<0.0001) were used.

FIG. 7 provides histograms of microglial phagocytosis of FITC-labeled amyloid beta (1-42) in the absence and presence of the indicated concentrations of 3AC (FIG. 7A), K118 (FIG. 7B) and K149 (FIG. 7C). Phagocytosis of beta amyloid by BV2 cells in the presence of K118, K149, K116 and K161 as compared to control are shown in FIG. 7D, and phagocytosis of beta amyloid by primary microglia incubated with K161 is shown in FIG. 7E. Confocal microscopic images visualize the amyloid phagocytosis by BV2 cells incubated with water (FIG. 7F and FIG. 7H, magnification) or with K161 (FIG. 7G and FIG. 7I, magnification).

FIG. 8 shows the effect of pan-SHIP1/2 inhibitors on mTor-pS6 signaling in the BV2 microglial cell line. Quantitation of the K161 effect on p-mTor expression (FIG. 8A) and fold increase of p-S6 expression (FIG. 8B). Western blotting results for various SHIP inhibitors on p-S6 are shown in FIG. 8C. Quantitation of the K118 effect on p-mTor expression is shown in

FIG. 8D. Each dose was analyzed in triplicate. (**p<0.01). The effect of compound 3AC on mTOR expression assessed by Western blotting as shown in FIG. 8E.

FIG. 9 shows the effect of an inhibitor of mTOR phosphorylation, Torin, on the K161-enhanced phagocytosis of dead neurons by BV2 cells (FIG. 9A), SIM-A9 cells (FIG. 9B), and primary microglial cells (FIG. 9C).

FIG. 10 shows the levels of expression of key signaling kinases AKT, (FIG. 10A); p-mTOR (FIG. 10B), and p-S6-PE (FIG. 10C), in cells incubated without and with K161. FIG. 10D shows the fold increase in p-S6 levels in cells incubated with K161.

FIG. 11 shows the effect of K118 on the number of CD11b+CD45+ myeloid cells in the CNS depicted in a flow cytometry plot (FIG. 11A) and line whisker plot (FIG. 11B). The lysosomal/phagosomal compartment is quantitated with lysotracker red for CD11b+CD45+ cells (FIG. 11C) and CD11b-CD45+ cells (FIG. 11D).

FIG. 12 shows the quantitation of microglial cells with the indicated markers (FIG. 12A-E) in mice treated with K161 for 2 weeks, assessed by flow cytometry. FIG. 12F-H are the density plots. Phagocytic activities of cells harvested from mice treated without and with K161is shown towards dead neurons (FIG. 12I), beta amyloid (FIG. 12J) and lysotracker (FIG. 12K).

FIG. 13 shows the detection of the K161 inhibitor in the brain of mice, 48 h and 96 h after administration of K161 by IP injection (mice 1-6) or oral gavage (mice 7-12) (FIG. 13A). Calibration curves for K161 are shown in FIGS. 13B and 13C, with experimental values added in FIG. 13D, from which the K161 concentrations were calculated (FIG. 13E).

FIG. 14 shows the detection of K118 and K161 in plasma after administration of a single dose of 10 mg/kg either IP or oral gavage administration.

### DETAILED DESCRIPTION

The present disclosure provides methods of activating microglial cells using inhibitors of the SH2-containing inositol 5′-phosphatase (SHIP).

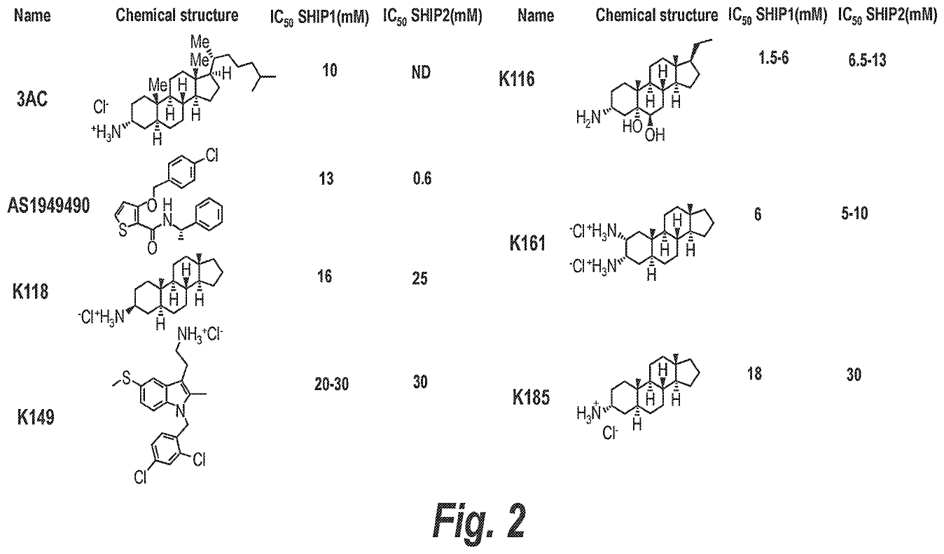

The “SHIP inhibitors” of the described herein are also referred to herein as “SHIP inhibitor compounds,” “SHIP1 inhibitors,” “SHIP1 inhibitor compounds,” “SHIP2 inhibitors,” SHIP2 inhibitor compounds,” “pan-SHIP1/2 inhibitors.” In particular, the disclosure describes the activation of microglial cells following exposure to pan-SHIP1/2 inhibitor compounds of Formulas I, II and III. In some embodiments, the compositions are selected from 3AC, K118, K149, K116, K161, and K185 (FIG. 2).

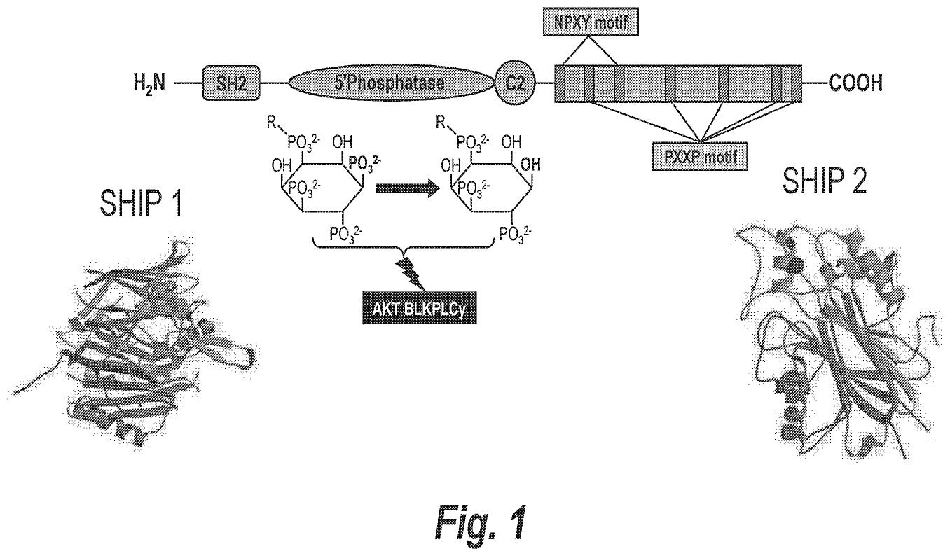

In some embodiments, the SHIP inhibitor compounds of the present invention are selective inhibitors of SHIP1 or SHIP2. In some embodiments, the SHIP inhibitor compounds of the present invention inhibit both SHIP1 and SHIP2. SHIP1 is a 145 kDa large protein and member of the inositol polyphosphate-5-phosphatase (INPP5) family. Alternate transcriptional splice variants, encoding different isoforms, have been characterized. At the N-terminus of the protein, SH2 domain is formed (FIG. 1). This domain is important for the interaction of SHIP1 with the phosphorylated protein chains that SHIP1 binds. Highly conserved phosphatase domain is in central part of the protein. This catalytic domain is flanked on the N-terminal side by the PH-like domain that binds phosphatidylinositol-3,4,5-triphosphate. SHIP1 and SHIP2 dephosphorylate inositol lipids at the 5D position, thereby generating PtdIns(3,4)P2 and activating the AKT signaling pathway (FIG. 1).

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, ±1%, and ±0.1% from the specified value, as such variations are appropriate to perform the disclosed methods.

As used herein, the term “alkyl group,” unless otherwise stated, refers to a C₁-C₄ branched or unbranched hydrocarbons. Examples of such alkyl groups include methyl groups, ethyl groups, propyl groups, butyl groups, isopropyl groups, sec-butyl, and tert-butyl groups. For example, the alkyl group can be a C₁-C₄ alkyl group including all integer numbers of carbons and ranges of numbers of carbons there between. The alkyl group can be unsubstituted or substituted with various substituents which may be the same or different.

The number of carbon atoms in an alkyl substituent can be indicated by the prefix “C_{x-y},” where x is the minimum and y is the maximum number of carbon atoms in the substituent. Likewise, a Cₓ chain means an alkyl chain containing x carbon atoms.

As used herein, the term “aryl group,” unless otherwise stated, refers to a C₅-C₆ aromatic carbocyclic group. The aryl group can be unsubstituted or substituted with various substituents which may be the same or different. A non-limiting example of a suitable aryl group includes phenyl.

As used herein, the term “heteroaryl” or “heteroaromatic” refers to a heterocycle having aromatic character. Heteroaryl substituents may be defined by the number of carbon atoms, e.g., C₁-C₉-heteroaryl indicates the number of carbon atoms contained in the heteroaryl group without including the number of heteroatoms. For example, a C₁-C₉-heteroaryl will include an additional one to four heteroatoms. Preferbably, the heteroaryl group has less than three heteroatoms. More preferably, the heteroaryl group has one to two heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂—CH₂—S(═O)—CH₃. Up to two heteroatoms may be consecutive, such as, for example, —CH₂—NH—OCH₃, or —CH₂—CH₂—S—S—CH₃. Preferred heteroalkyl groups have 1-12 carbons.

As used herein, the term “alkenyl,” denotes a monovalent group derived from a hydrocarbon moiety containing at least two carbon atoms and at least one carbon-carbon double bond. The double bond may or may not be the point of attachment to another group. Alkenyl groups (e.g., C₂-C₈-alkenyl) include, but are not limited to, for example, ethenyl, propenyl, prop-1-en-2-yl, butenyl, 1-methyl-2-buten-1-yl, heptenyl, octenyl and the like.

As used herein, the term “halo group,” unless otherwise stated, refers to fluoro, chloro, bromo and iodo.

As used herein, the term “haloalkyl” refers to alkyl radicals wherein any one or more of the alkyl carbon atoms is substituted with halo as defined above. Haloalkyl embraces monohaloalkyl, dihaloalkyl, and polyhaloalkyl radicals. The term “haloalkyl” includes, but is not limited to, fluoromethyl, difluoromethyl, trifluoromethyl, chloromethyl, dichloromethyl, trichloromethyl, and pentafluoroethyl.

As used herein, the term “cycloalkyl” refers to a mono cyclic or polycyclic non-aromatic radical, wherein each of the atoms forming the ring (i.e., skeletal atoms) is a carbon atom. In one embodiment, the cycloalkyl group is saturated or partially unsaturated. In another embodiment, the cycloalkyl group is fused with an aromatic ring. Cycloalkyl groups include groups having 3 to 10 ring atoms (C₃-C₁₀-cycloalkyl), groups having 3 to 8 ring atoms (C₃-C₈-cycloalkyl), groups having 3 to 7 ring atoms (C₃-C₇-cycloalkyl), and groups having 3 to 6 ring atoms (C₃-C₆-cycloalkyl). Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes unsaturated nonaromatic cyclic groups, which contain at least one carbon carbon double bond or one carbon carbon triple bond.

As used herein, the term “heterocycloalkyl” or “heterocyclyl” refers to a heteroalicyclic group containing one to four ring heteroatoms each selected from O, S, and N. In one embodiment, each heterocyclyl group has from 3 to 10 atoms in its ring system, with the proviso that the ring of said group does not contain two adjacent O or S atoms. Heterocyclyl substituents may be alternatively defined by the number of carbon atoms, e.g., C₂-C₈-heterocyclyl indicates the number of carbon atoms contained in the heterocyclic group without including the number of heteroatoms. For example, a C₂-C₈-heterocyclyl will include an additional one to four heteroatoms. Preferbably, the heterocyclyl group has less than three heteroatoms. More preferably, the heterocyclyl group has one to two heteroatoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure.

An example of a 3-membered heterocyclyl group includes, and is not limited to, aziridine. Examples of 4-membered heterocycloalkyl groups include, and are not limited to, azetidine and a beta lactam. Examples of 5-membered heterocyclyl groups include, and are not limited to, pyrrolidine, oxazolidine and thiazolidinedione. Examples of 6-membered heterocycloalkyl groups include, and are not limited to, piperidine, morpholine, and piperazine.

Other non-limiting examples of heterocyclyl groups include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “spiro-heterocycle” refers to bicyclic structures that may be bridged or spirocyclic in nature with each individual ring within the bicycle varying from 3-8 atoms, and containing 0, 1, or 2 N, O, or S atoms. The term also specifically includes, but is not limited to, 6-oxa-3-azabicyclo[3.1.1]heptanyl, 2-azaspiro[3.3]heptanyl, 2-oxa-6-azaspiro[3.3]heptanyl, 2-oxaspiro[3.3]heptanyl, 2-oxaspiro[3.5]nonanyl, 3-oxaspiro[5.3]nonanyl, and 8-oxabicyclo[3.2. 1]octanyl, and the like.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

As used herein, the terminology “selected from . . . ” (e.g., “R⁴ is selected from A, B and C”) is understood to be equivalent to the terminology “selected from the group consisting of . . . ” (e.g., “R⁴ is selected from the group consisting of A, B and C”).

The term “inositol polyphosphate 5-phosphatase” as used herein refers to a family of phosphatases each of which removes the 5′ phosphate from inositol- and phosphatidylinositol-polyphosphates.

The term “SHIP” as used herein refers to SH2-containing inositol-5-phosphatase. SHIP may have an apparent molecular weight of about 145 kDa and is expressed in at least hemopoietic cells. It contains an amino-terminal src-homology domain (SH2), a central 5′-phosphoinositol phosphatase domain, two phosphotyrosine binding consensus sequences, and a proline-rich region at the carboxyl tail.

The term “SHIP1” as used herein refers to a SHIP protein isoform encoded by the gene INPPSD (Accession No. NG_033988.1). SHIP1 has two protein isoforms, an “a” isoform of 1189 amino acids (Accession No. NP_001017915.1) and a “b” isoform of 1188 amino acids (Accession No. NP_005532.2). SHIP1 is expressed by hematopoietic-derived cells, osteoblasts, and mesenchymal cells. SHIP1 has been shown to act as a negative controller in immunoreceptor signaling, as a negative controller in hematopoietic progenitor cell proliferation and survival, and as an inducer of cellular apoptosis (Viernes et al., Discovery and development of small molecule SHIP phosphatase modulators. Med Res Rev. (2014); 34(4): 795-824, incorporated herein by reference in its entirety).

The term “SHIP2” as used herein refers to a SHIP protein isoform of 1258 amino acids (Accession No. NP_001558.3) encoded by the gene INPPL1 (Accession No. NG_023253.1). SHIP2 is expressed across all cell and tissue types, with high levels of SHIP2 being expressed in the heart, skeletal muscle, and placenta. SHIP2 has been shown to be a negative regulator of the insulin-signaling pathway. (Viernes et al., Discovery and development of small molecule SHIP phosphatase modulators. Med Res Rev. (2014); 34(4): 795-824).

The term “protecting group” or “chemical protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, monomethoxytrityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid moieties may be blocked with base labile groups such as, without limitation, methyl, or ethyl, and hydroxy reactive moieties may be blocked with base labile groups such as acetyl in the presence of amines blocked with acid labile groups such as tert-butyl carbamate or with carbamates that are both acid and base stable but hydrolytically removable.

As described herein, the term “cell proliferation” refers to an increase in the number of cells. Cell proliferation is the process that results in a net increase of the number of cells, and is defined by the balance between cell divisions and cell loss through cell death or differentiation.

As described herein, the term “cell death” refers to when a cell ceases to carry out its functions. Cell death may be the result of the natural process of old cells dying and being replaced by new ones, or may result from such factors as disease, localized injury, or the death of the organism of which the cells are part. Apoptosis or Type I cell-death, and autophagy or Type II cell-death are both forms of programmed cell death, while necrosis is a process that often occurs as a result of infection or injury.

As described herein, the term “phagocytosis” refers to the process by which a cell uses its plasma membrane to engulf an object giving rise to an internal compartment called the phagosome. Phagocytosis is a highly complex and specialized process directed at the uptake and removal of opsonized and non-opsonized targets, such as pathogens, apoptotic cells, and cellular debris. Phagocytosis is also present throughout early neural development, homeostasis, and initiating repair mechanisms. To assist in maintaining homeostasis in the CNS, synapses, apoptotic cells, and debris must be continuously removed. Phagocytosis in the CNS is primarily attributed to microglia. Other, phagocytes (e.g., astrocytes or oligodendrocytes) may also participate to phagocytosis. Arising from discrete pathologies, insoluble protein aggregates such as those comprising amyloid protein, or myelin debris and apoptotic or dead neurons are among the specific phagocytic targets.

As used herein, the term “lysosomal/phagosomal” compartment of a cell refers to the collective space in a cell occupied by lysosomes and/or phagolysosomes. Lysosomes are vesicles in a cell that contain hydrolytic enzymes that can break down many kinds of biomolecules. The phagosome is the organelle formed by phagocytosis of material. It then moves toward the centrosome of the phagocyte and is fused with lysosomes, forming a phagolysosome. Progressively, the phagolysosome is acidified, activating degradative enzymes, and leading to degradation of ingested materials.

As used herein, the term “treatment” or “treating” is defined as the application or administration of a therapeutic agent, i.e., a SHIP1 inhibitor (alone or in combination with another pharmaceutical agent), to a patient, or application or administration of a therapeutic agent.

A “therapeutically effective amount” describes an amount that will generate the desired therapeutic outcome (i.e., achieve therapeutic efficacy). For example, a therapeutically effective dose of a compound of the present disclosure is an amount that is sufficient to palliate, ameliorate, stabilize, reverse, prevent, slow or delay the progression of the disease state (e.g., Alzheimer's disease). A therapeutically effective amount can be an amount administered in a dosage protocol that includes days or weeks of administration.

As used herein, the term “patient,” “individual” or “subject” refers to a human or a non-human mammal. Non-human mammals include, for example, livestock and pets, such as ovine, bovine, porcine, canine, feline and murine mammals. Non-human mammals also include non-human primates, rats, rabbits and camelids. In certain embodiments, the patient, subject, or individual is human.

In one aspect, the disclosure provides a kit for treating Alzheimer's disease in an individual. The kit comprises a compound of the present disclosure, pharmaceutically acceptable esters, salts, and prodrugs thereof, and instructions for use. The instructions may include details on one or more of the following: dosage, frequency, number of administrations to be carried out (such as number of tablets to be consumed), whether the composition needs to be taken with food, water etc., storage of the composition, and the like.

Compounds of the disclosure can exist as salts. Pharmaceutically acceptable salts of the compounds of the disclosure generally are preferred in the methods of the disclosure. As used herein, the term “pharmaceutically acceptable salts” refers to salts or zwitterionic forms of a compound of the present disclosure. Salts of compounds of the present disclosure can be prepared during the final isolation and purification of the compounds or separately by reacting the compound with an acid having a suitable cation. The pharmaceutically acceptable salts of a compound of the present disclosure are acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds of the present disclosure appearing herein is intended to include a compound of the present disclosure as well as pharmaceutically acceptable salts, hydrates, or prodrugs thereof.

Prodrugs of a compound of the present disclosure also can be used as the compound in a method of the present disclosure. Compounds of the present disclosure can contain one or more functional groups. The functional groups, if desired or necessary, can be modified to provide a prodrug. Suitable prodrugs include, for example, acid derivatives, such as amides and esters. It also is appreciated by those skilled in the art that N-oxides can be used as a prodrug.

Compositions comprising a compound of the disclosure and a pharmaceutical agent can be prepared at a patient's bedside, or by a pharmaceutical manufacture. In the latter case, the compositions can be provided in any suitable container, such as a sealed sterile vial or ampoule, and may be further packaged to include instruction documents for use by a pharmacist, physician or other health care provider. The compositions can be provided as a liquid, or as a lyophilized or powder form that can be reconstituted if necessary when ready for use. In particular, the compositions can be provided in combination with any suitable delivery form or vehicle, examples of which include, for example, liquids, caplets, capsules, tablets, inhalants or aerosol, etc. The delivery devices may comprise components that facilitate release of the pharmaceutical agents over certain time periods and/or intervals, and can include compositions that enhance delivery of the pharmaceuticals, such as nanoparticle, microsphere or liposome formulations, a variety of which are known in the art and are commercially available. Further, each composition described herein can comprise one or more pharmaceutical agents. The compositions described herein can include one or more standard pharmaceutically acceptable carriers. Some examples of pharmaceutically acceptable carriers can be found in: Remington: The Science and Practice of Pharmacy (2005) 21st Edition, Philadelphia, PA. Lippincott Williams & Wilkins.

The amount of provided compounds that may be combined with carrier materials to produce a composition in a single dosage form will vary depending upon the patient to be treated and the particular mode of administration. The compositions may be formulated such that a desired dosage of the inhibitor can be administered to a patient receiving these compositions.

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention. The compositions can be used in conjunction with any other conventional treatment modality designed to improve the disorder for which a desired therapeutic or prophylactic effect is intended, non-limiting examples of which include surgical interventions and radiation therapies. The compositions can be administered once, or over a series of administrations at various intervals determined using ordinary skill in the art, and given the benefit of the present disclosure.

Direct CNS administration or targeting is not required for the effects of SHIP inhibitors described herein. In some embodiments, a SHIP inhibitor is administered peripherally to a subject. In an embodiment, such administration is pulsatile or continuous for 2, 3, 4, 5, 6, 7, 8, 9, 10 or more days. In an embodiment, such administration is pulsatile or continuous for at least 2, 3, 4, 5, 6, 7, 8, 9, 10 or 11 days. Administration of the treatment can be other than peripherally, such as directly into the CNS.

In some embodiments, multiple doses of the compounds and salts described herein may be administered to a subject in a pulsatile or intermittent manner. As used herein the term “pulsatile dose regimen” or “intermittent dose regimen” refers to a dose administration regimen which includes at least two dosing cycles. Each subsequent dosing cycle is separated by a rest period from the preceding dosing cycle. As used herein, the term “continuous dosing” refers to dosing without a rest period between dosing periods.

As used herein in reference to a pulsatile dose regimen, the term “dosing cycle” refers to a single cycle of dose administration that can be repeated, with each dosing cycle consisting of one or more dose administrations of the compound, salt or solvate at a set dosage for a set administration period at set time intervals during a defined period of time (e.g., 50 mg/kg of compound administered for 1 hour every 8 hours over a period of two days). The dosage and administration period of each dose administration of a dosing cycle, and of different dosing cycles in a pulsatile dose regimen, can be the same or can differ. The time intervals between the dose administrations of a dosing cycle can be the same or can differ (e.g., the first two of three dose administrations can be 8 hours apart and the third dose administration can be 24 hours later). The duration of each dosing cycle in a pulsatile dose regimen can be the same or can differ (e.g., the first dosing cycle of three can have a duration of three days, the second dosing cycle can have a duration of two days and the third dosing cycle can have a duration of one day). Example dosages that can be delivered in each dose administration, dosing cycle and pulsatile dose regimen are described below. In some embodiments, the administration period of any dose administration of a dosing cycle has a duration of about 1/10, ⅙, ¼, ½, 1, 2, 3, 4, 5, 6, 7, 8, 9, 10, 11, 12, 15, 18, 21, 24, 36 or 48 hours, or any time period in between. In some embodiments, the time interval between two dose administrations of a dosing cycle is about 1, 2, 3, 4, 5, 6, 7, 8, 9, 10, 11, 12, 15, 18, 21, 24, 36, or 48 hours, or any time period in between. In various embodiments, the time interval can define as an interval of time between the start time of successive dose administrations or between the end of one dose administration and the start of the next. In some embodiments, the time intervals between dose administrations of a dosing cycle may be expressed in terms of the number of dose administrations per day, and include administration of a dose 2, 3, 4, 5, 6, 7, 8, 9, 10, 11, 12, 18 or 24 times a day. In some embodiments, the time intervals for dose administrations of a dosing cycle may be expressed as a specified number of administrations at specified times of the day, such as, for example, four dose administrations per day given at 8:00 am, 12:00 pm, 4:00 pm, and 8:00 pm, or three dose administrations two hours after each of three meals. In some embodiments, a dosing cycle includes time intervals between dose administrations of 1-3 hours, 3-6 hours, 6-9 hours, 9-12 hours, 12-15 hours, 15-18 hours, 18-21 hours, or 21-24 hours. In some embodiments, the defined period of time during which the dose administrations of a dosing cycle are made (i.e., the length of a dosing cycle) is 1, 2, 3, 4, 5, 6, 7, 8, 9, 10, 11, 12, 15, 18, 21, 24, or 28 days. In some embodiments, the dosage of the dose administrations during a dosing cycle may vary due to the different lengths of the administration periods. For example, a first administration period of a solvate may be 3 hours, while a second administration period of the solvate may be 1 hour, resulting in a lower dosage of the solvate being administered. In some embodiments, each of the one or more dose administrations during a dosing cycle may deliver the same or a different dosage of the compound, salt or solvate, or use the same or a different concentration.

As used herein when referring to a pulsatile dose regimen, the term “rest period” refers to a period of time during which no doses of the compound, salt, or solvate thereof are administered. During a pulsatile dose regimen, each succeeding dosing cycle is separated from the immediately preceding dosing cycle by a rest period. For example, if the pulsatile dose regimen consists of three dosing cycles, there is a rest period between the first dosing cycle and second dosing cycle and between the second dosing cycle and the third dosing cycle. In some embodiments, the rest period is 1, 2, 3, 4, 5, 6, 7, 8, 9, 10, 11, 12, 15, 18, 21, 24, or 28 days, or 5, 6, 7, 8, 9, 10, 11 or 12 weeks. In some embodiments, one or more of the rest periods in a pulsatile dose regime differ in their length of time. For example, a first rest period of a pulsatile dose regimen may be three days and subsequent rest periods may be 5 days.

In some embodiments, SHIP inhibitors are formulated in dosage unit form for ease of administration and uniformity of dosage. “Dosage unit form” as used herein refers to physically discrete units suited as unitary dosages for the patients to be treated; each unit containing a predetermined quantity of the SHIP inhibitor calculated to produce the desired therapeutic effect in association with the required pharmaceutical vehicle. The dosage unit forms of the invention are dictated by and directly dependent on (a) the unique characteristics of the SHIP inhibitor and the particular therapeutic effect to be achieved, and (b) the limitations inherent in the art of compounding/formulating such a SHIP inhibitor for the inhibition of SHIP in a patient.

In one embodiment, the compounds of the invention are formulated using one or more pharmaceutically acceptable excipients or carriers. In one embodiment, the pharmaceutical compositions of the invention comprise a therapeutically effective amount of a SHIP inhibitor and a pharmaceutically acceptable carrier.

In some embodiments, the dose of a SHIP inhibitor is from about 0.05 mg/kg to about 150 mg/kg and particularly in a dosing range of from about 0.1 mg/kg to about 100 mg/kg. More particularly, the dosing range can be from 0.08 mg/kg to 140 mg/kg, from 0.1 mg/kg to 130 mg/kg, from 0.1 mg/kg to 120 mg/kg, from 0.1 mg/kg to 110 mg/kg, from 0.1 mg/kg to 110 mg/kg, from 0.5 mg/kg to 100 mg/kg, from 1 mg/kg to 100 mg/kg, from 10 mg/kg to 80 mg/kg, from 20 mg/kg to 70 mg/kg, from 20 mg/kg to 60 mg/kg, from 20 mg/kg to 50 mg/kg, from 20 mg/kg to 40 mg/kg, and from 20 mg/kg to 30 mg/kg.

Different dosage regimens may be used to inhibit SHIP. In one embodiment, the compounds of the invention are administered at a dose from 0.05 mg/kg to 150 mg/kg or more particularly at a dose from 0.1 mg/kg to 100 mg/kg once a day, every other day, three times a week, twice a week, once a week, etc. In another embodiment, the SHIP inhibitors described herein are administered at a dose from 0.08 mg/kg to 140 mg/kg, from 0.1 mg/kg to 130 mg/kg, from 0.1 mg/kg to 120 mg/kg, from 0.1 mg/kg to 110 mg/kg, from 0.1 mg/kg to 110 mg/kg, from 0.5 mg/kg to 100 mg/kg, from 1 mg/kg to 100 mg/kg, from 10 mg/kg to 80 mg/kg, from 20 mg/kg to 70 mg/kg, from 20 mg/kg to 60 mg/kg, from 20 mg/kg to 50 mg/kg, from 20 mg/kg to 40 mg/kg, and from 20 mg/kg to 30 mg/kg once a day, every other day, three times a week, twice a week, once a week, etc. In some embodiments, the dosage form is provided in a packaged pharmaceutical composition comprising a container holding a therapeutically effective amount of a SHIP1 inhibitor, alone or in combination with a second pharmaceutical agent; and instructions for using the compound to inhibit SHIP in a patient.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans) urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the SHIP inhibitors may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The pharmaceutical compositions may be provided in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

Identifying an individual in need of treatment can be in the judgment of a health care professional and can be subjective (e.g. opinion) or objective (e.g. measurable by a test or diagnostic method). In other methods, the individual is prescreened or identified as in need of such treatment by assessment for a relevant marker or indicator of suitability for such treatment.

For human use, a compound of the present disclosure can be administered alone, but generally is administered in admixture with a pharmaceutical carrier selected with regard to the intended route of administration and standard pharmaceutical practice. Pharmaceutical compositions for use in accordance with the present disclosure can be formulated in a conventional manner using one or more physiologically acceptable carrier comprising excipients and auxiliaries that facilitate processing of a compound of the present disclosure into pharmaceutical preparations.

For veterinary use, a compound of the present disclosure, or a pharmaceutically acceptable salt or prodrug, is administered as a suitably acceptable formulation in accordance with normal veterinary practice. The veterinarian can readily determine the dosing regimen and route of administration that is most appropriate for a particular animal. Animals treatable by the present compounds and methods include, but are not limited to, bovines or ungulates.

The present compounds may be used with pharmaceutically acceptable carriers, which may be solvents, suspending agents, vehicles or the like for delivery to humans or animals. The carrier may be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Examples of pharmaceutically-acceptable carriers include pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

The present compositions may be administered by any suitable route-either alone or as in combination with other therapeutic or non-therapeutic agents. Administration can be accomplished by any means, such as, for example, by parenteral, mucosal, pulmonary, topical, catheter-based, or oral means of delivery. Parenteral delivery can include, for example, subcutaneous, intravenous, intramuscular, intra-arterial, and injection into the tissue of an organ. Mucosal delivery can include, for example, intranasal delivery. Pulmonary delivery can include inhalation of the agent. Catheter-based delivery can include delivery by iontophoretic catheter-based delivery. Oral delivery can include delivery of an enteric coated pill, or administration of a liquid by mouth.

When administered in combination with other therapeutics, a present compound may be administered at relatively lower dosages. In addition, the use of targeting agents may allow the necessary dosage to be relatively low. Certain compounds may be administered at relatively high dosages due to factors including, but not limited to, low toxicity and high clearance.

Other than in the operating examples, or unless otherwise expressly specified, all of the numerical ranges, amounts, values and percentages such as those for amounts of materials, times and temperatures of reaction, ratios of amounts, values for molecular weight (whether number average molecular weight (“Mn”) or weight average molecular weight (“Mw”), and others in the specification may be read as if prefaced by the word “about” even though the term “about” may not expressly appear with the value, amount or range. Numerical parameter should be construed in light of the number of reported significant digits and by applying ordinary rounding techniques.

Also provided herein are methods of activating microglial cells with a SHIP inhibitor. In some embodiments, the inhibitor is a pan-SHIP1/2 inhibitor. In some embodiments, the inhibitor has a structure of Formula I, II or III. The activation of microglial cells can be used to treat neurodegenerative disorders in subjects in need thereof. In some embodiments, the neurodegenerative disorder is Alzheimer's disease.

As described herein, Alzheimer's disease Alzheimer's disease (AD), also referred to as Alzheimer's, is a chronic neurodegenerative disease. Alzheimer's disease is characterized by the loss of neurons and synapses in the cerebral cortex and certain subcortical regions. This loss results in a significant atrophy of the affected regions in the brain, including degeneration in the temporal lobe and parietal lobe, and parts of the frontal cortex and cingulate gyrus. In brains from patients afflicted with CD both amyloid plaques and neurofibrillary tangles are visible microscopically. Plaques are dense, and for the most part insoluble, deposits of beta-amyloid peptide and cellular material, and forms outside and around the neurons. Alzheimer's disease has been identified as a protein misfolding disease (proteopathy), caused by plaque accumulation of abnormally folded amyloid beta protein and tau protein in the brain. Plaques are made up of small peptides, 39-43 amino acids in length, called amyloid beta. Amyloid beta is a fragment from the larger amyloid precursor protein. Amyloid precursor protein is a transmembrane protein that penetrates through the neuron's membrane. The genetic heritability of Alzheimer's disease ranges from 49% to 79%. Most of autosomal dominant familial Alzheimer's disease can be attributed to mutations in one of three genes: those encoding amyloid precursor protein and presenilins 1 and 2. Most mutations in the amyloid precursor protein and presenilin genes increase the production of a small protein called Aβ42, which is the main component of senile plaques.

As described herein, amyloids are aggregates of proteins that become folded into a shape that allows many copies of that protein to stick together, forming fibrils. In the human body, amyloids have been linked to the development of various diseases. Pathogenic amyloids form when previously healthy proteins lose their normal physiological functions and form fibrous deposits in plaques around cells which can disrupt the healthy function of tissues and organs. Such amyloids have been associated with (but not necessarily as the cause of) more than 50 human diseases, known as amyloidosis, and may play a role in some neurodegenerative disorders. In the human body, amyloids have been linked to the development of various diseases. Pathogenic amyloids form when previously healthy proteins lose their normal physiological functions and form fibrous deposits in plaques around cells which can disrupt the healthy function of tissues and organs. Such amyloids have been associated with (but not necessarily as the cause of) more than 50 human diseases, known as amyloidosis, and may play a role in some neurodegenerative disorders. The neurodegenerative disorders involving amyloids (with the amyloid mentioned in parentheses) are: Alzheimer's disease (Beta amyloid from Amyloid precursor protein), diabetes mellitus type 2 (IAPP or Amylin), Parkinson's disease (Alpha-synuclein), transmissible spongiform encephalopathy, e.g. bovine spongiform encephalopathy (PrPSc, APrP), fatal familial insomnia (PrPSc APrP), Huntington's disease (Huntingtin), medullary carcinoma of the thyroid (Calcitonin), cardiac arrhythmias, isolated atrial amyloidosis (Atrial natriuretic factor), atherosclerosis (Apolipoprotein AI), rheumatoid arthritis (Serum amyloid A), aortic medial amyloid (Medin), prolactinomas (Prolactin), familial amyloid polyneuropathy (Transthyretin), hereditary non-neuropathic systemic amyloidosis (Lysozyme), dialysis related amyloidosis (Beta-2 microglobulin), Finnish amyloidosis (Gelsolin), lattice corneal dystrophy (Keratoepithelin), cerebral amyloid angiopathy (Beta amyloid), cerebral amyloid angiopathy, Icelandic type (Cystatin), systemic AL amyloidosis (Immunoglobulin light chain AL) and sporadic Inclusion body myositis (S-IBM).

As described herein, microglial cells, microglia, or microglia cells, refer to the resident macrophage cells of the brain. Microglial cells act as the first and main form of active immune defense in the central nervous system (CNS). Microglia are important for overall brain maintenance since they are constantly scavenging the CNS for plaques, damaged or unnecessary neurons and synapses, and infectious agents. The main role of microglia, is phagocytosis, and involves the engulfing of various materials. Engulfed materials generally consist of cellular debris, lipids, and apoptotic cells in the non-inflamed state, and invading virus, bacteria, or other foreign materials in the inflamed state.

As described herein “CD” followed by a number refers to the cluster of differentiation (also known as cluster of designation or classification determinant and often abbreviated as CD) used for the identification and investigation of cell surface molecules providing targets for immunophenotyping of cells. For Example, CD45 refers to cluster of differentiation protein number 45 which is expressed on hematopoietic cells and represents multiple isoforms of a tyrosinephosphatase. CD11b refers to cluster of differentiation protein number 11b representing an AlphaM integrin chain expressed on myeloid cells. Resting microglia are CD11b+CD45 low, meaning that they express high levels of CD11b, and low levels of CD45. Other macrophages can be distinguished from microglial cells on the basis that they are CD11b+CD45+, meaning that they express high levels of both CD11b and CD45. As used herein, ACSA means astrocyte cell surface antigen-2, which is expressed on astrocytes, but not on microglial cells.

Microglia act as an immune defense in the central nervous system (CNS). Due to the shared lineage of microglia and macrophages, many markers are common to both cell types. A combination of CD11b and CD45 labeling can be used to distinguish microglia from macrophages. Resting microglia are CD11b+CD45 low, whereas macrophages are CD11b+CD45+. Further, ACSA-2 is an anti-astrocyte cell surface antigen-2, and the anti-ACSA-2 antibody shows substantially less labeling of microglial cells.

Provided herein are methods of increasing the number of CD11b+CD45+ myeloid cells in the central nervous system (CNS) with a SHIP inhibitor. In some embodiments, the inhibitor is a pan-SHIP1/2 inhibitor. In some embodiments, the inhibitor has a structure of Formula I, II or III. The activation of these myeloid cells can be used to treat neurodegenerative disorders in subjects in need thereof. In some embodiments, the myeloid cells are macrophages. In some embodiments, the neurodegenerative disorder is Alzheimer's disease.

Provided herein are methods of increasing the lysosomal/phagosomal compartment size of CD11b+CD45 low myeloid cells in the brain with a SHIP inhibitor. In some embodiments, the inhibitor is a pan-SHIP1/2 inhibitor. In some embodiments, the inhibitor has a structure of Formula I, II or III. The activation of hematopoietic cells can be used to treat neurodegenerative disorders in subjects in need thereof. In some embodiments, the hematopoietic cells are microglial cells. In some embodiments, the neurodegenerative disorder is Alzheimer's disease.

Provided herein are methods of increasing the lysosomal/phagosomal compartment size of CD11b+CD45+ hematopoietic cells in the CNS with a SHIP inhibitor. In some embodiments, the inhibitor is a pan-SHIP1/2 inhibitor. In some embodiments, the inhibitor has a structure of Formula I, II or III. The activation of myeloid cells can be used to treat neurodegenerative disorders in subjects in need thereof. In some embodiments, the myeloid cells are macrophages. In some embodiments, the neurodegenerative disorder is Alzheimer's disease.

The following examples are presented to illustrate the present disclosure. They are not intended to be limiting in any manner.

### EXAMPLES

### Example 1: SHIP1 and SHIP2 Are Expressed on Microglial Cell Lines and Primary Microglia Cells

Both SHIP1 and SHIP2 have been shown to be expressed by peripheral myeloid cells including terminally differentiated macrophages. SHIP1 influences signaling downstream of the following receptors elaborated by terminally differentiated myeloid cells: TREM2, Dectin1, FcγR, c-Kit, G-CSF-R and CCR2 while SHIP2 has been shown to regulate M-CSF and FcγR signaling. Thus, SHIP1 and/or SHIP2 could also play a role in cell signaling pathways that control microglial proliferation, survival, effector functions and/or chemotaxis.

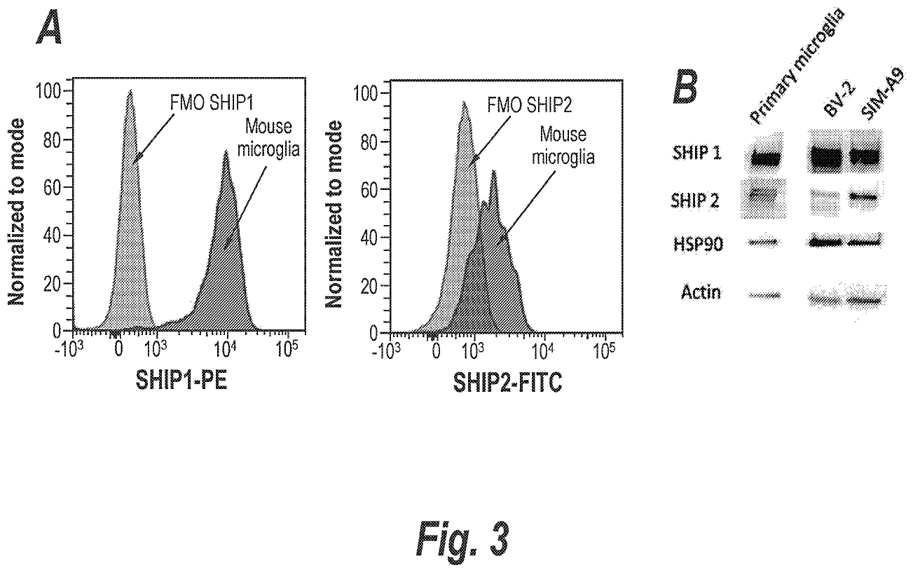

Assessment as to whether one or both paralogs are expressed was done by flow cytometry (FIG. 3A) and by Western blot (FIG. 3B). BV2, SIM-A9 or primary microglial cells were labeled with an antibody specifically recognizing microglial cells. For the Western blotting experiment, blots were incubated with an antibody recognizing SHIP1, or SHIP2. SHIP I was detected with Santa Cruz antibody clone P1C1 antibody, while for SHIP2 Santa Cruz (E-2) was used. Different portions of the Western blot were then stripped, and reprobed for expression of two ubiquitously expressed proteins (HSP90 or actin) to provide an internal control for loading of cellular protein. Experiments were conducted with extracts from microglial cell lines, and with primary microglial cells. The primary mouse microglial cells express both SHIP1 and SHIP2 (FIG. 3A), which was confirmed by Western Blotting (FIG. 3B). Cell lines BV2 and SIM-A9 also expressed both SHIP paralogs. The results obtained suggest that both paralogs could potentially influence cell signaling pathways in microglia.

### Example 2: SHIP Inhibitors Promote the Proliferation Of Microglia

The ability of 3AC, K118 and K149 to promote the proliferation of BV2 and SIM-A9 microglial cells was tested in vitro. Cells were counted with a cell counting kit-8 (e.g. CCK-8) which is a sensitive colorimetric assays for the determination of cell viability in cell proliferation and cytotoxicity assays. It uses the highly water-soluble tetrazolium salt, WST-8, which is reduced by dehydrogenase activities in cells to give a yellow-color formazan dye, which is soluble in the tissue culture media. The amount of the formazan dye, generated by the activities of dehydrogenases in cells, is directly proportional to the number of living cells.

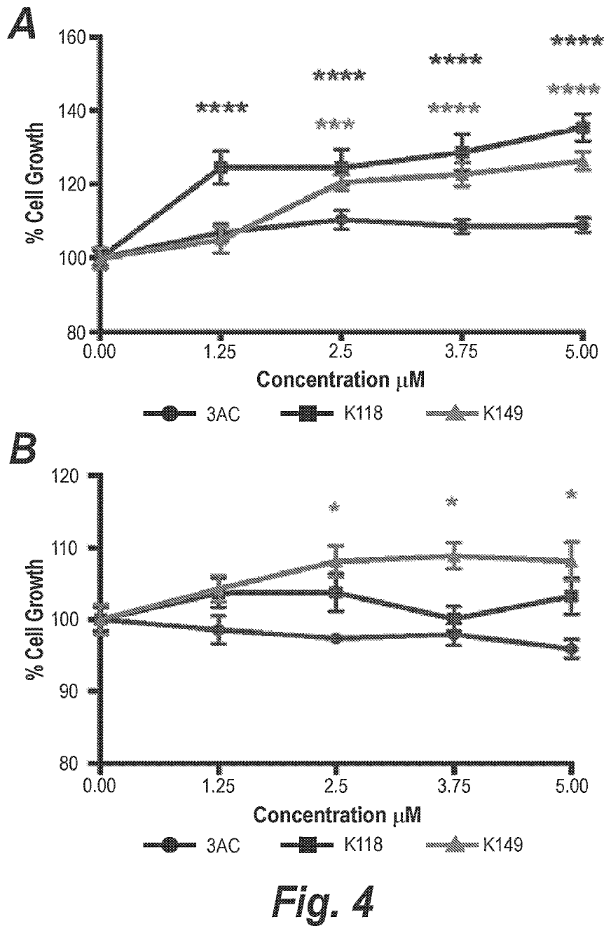

A CCK-8 assay was performed for analysis of cell growth of BV2 cells (FIG. 4A), and SIM-A9 cells (FIG. 4B) vs. their respective vehicle controls (100%) at 48 hr. Each point represents the mean of the 6 replicates for each dose. K118 showed a significant increase in cell growth of BV2 cells at all concentrations shown tested. K149 showed effects at 2.5, 3.75 and 5 μM concentrations (p<0.05, unpaired Student's T-Test). For the SIM-A9 cell line, an effect on proliferation was observed for K149, at 2.5-5 μM concentrations (FIG. 4B). Across three independent experiments it was observed that K118 has the most potent impact on microglial growth relative to either K149 or 3AC. Taken together these results indicate SHIP inhibitors can be used to promote microglial proliferation.

### Example 3: SHIP Inhibitors Increase Microglial Phagosomal Compartment Size

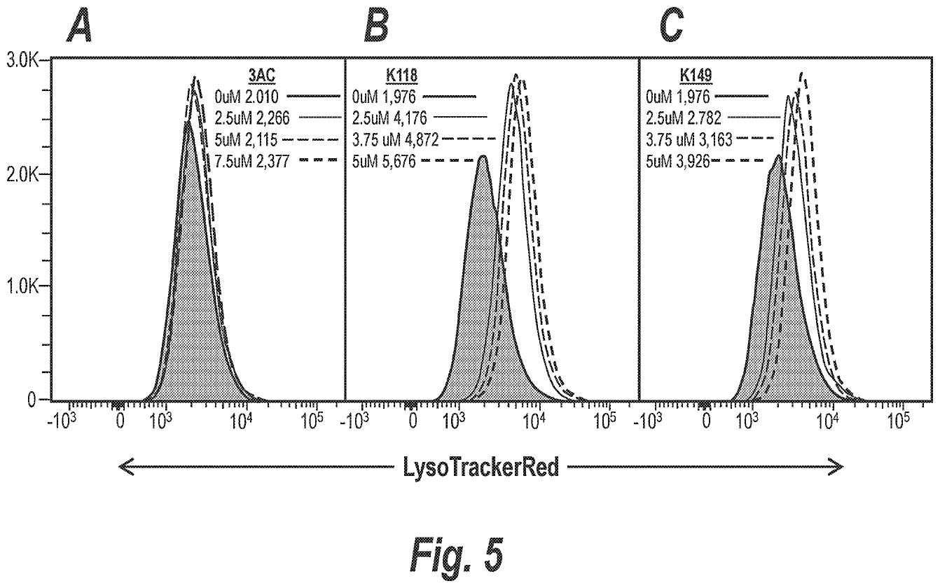

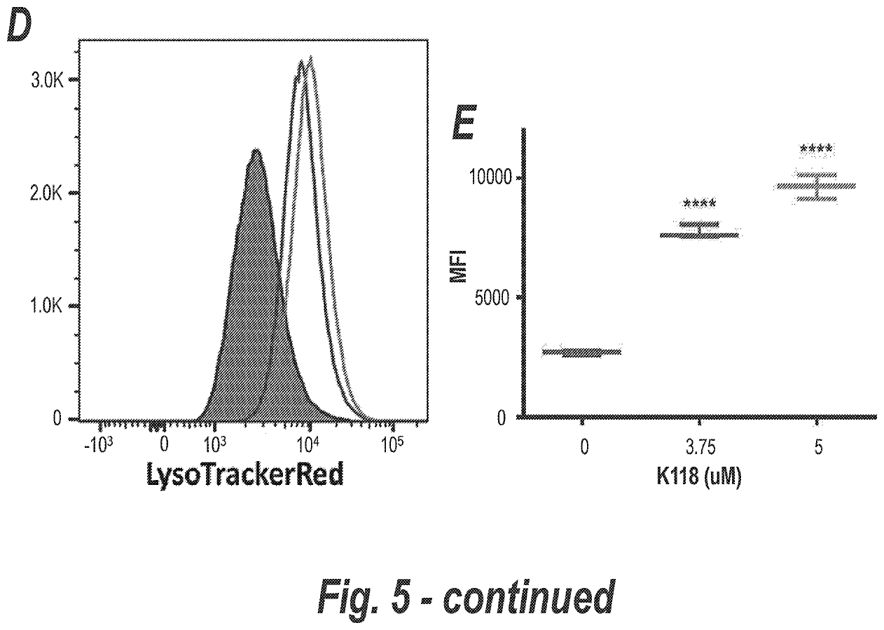

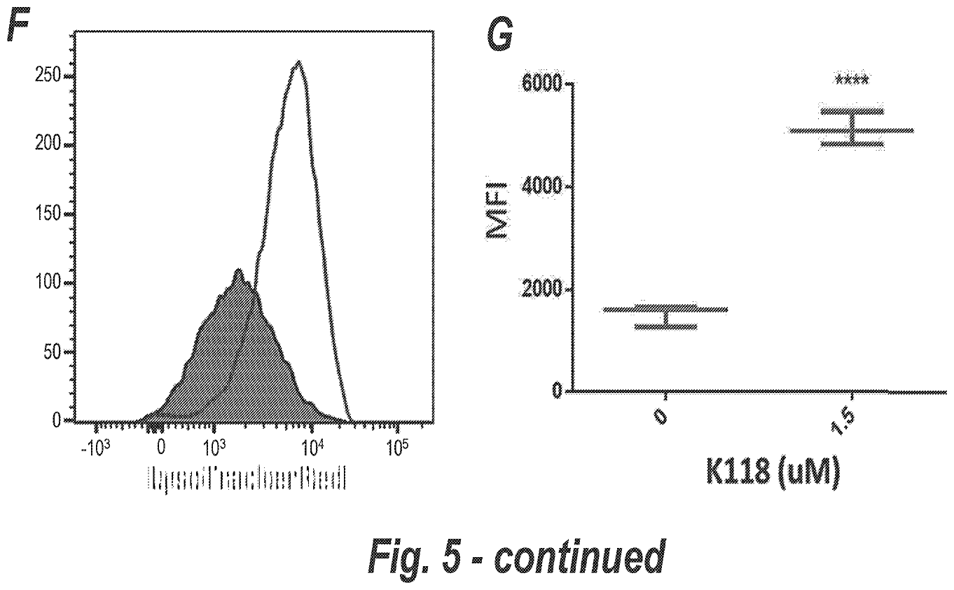

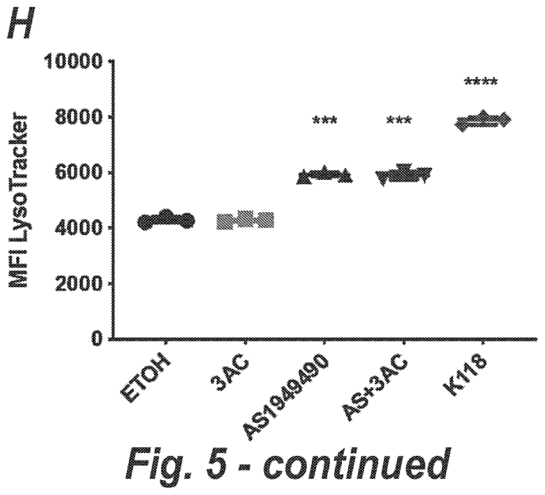

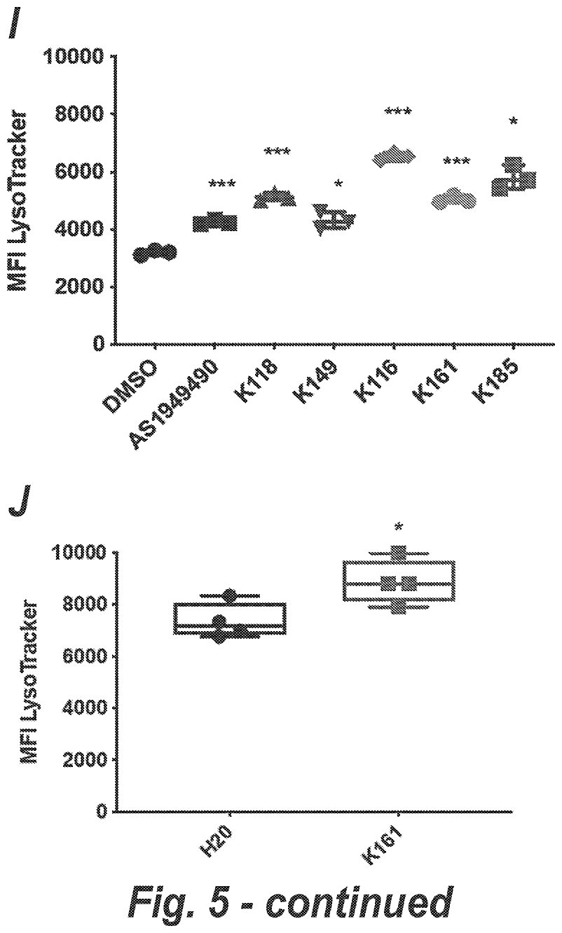

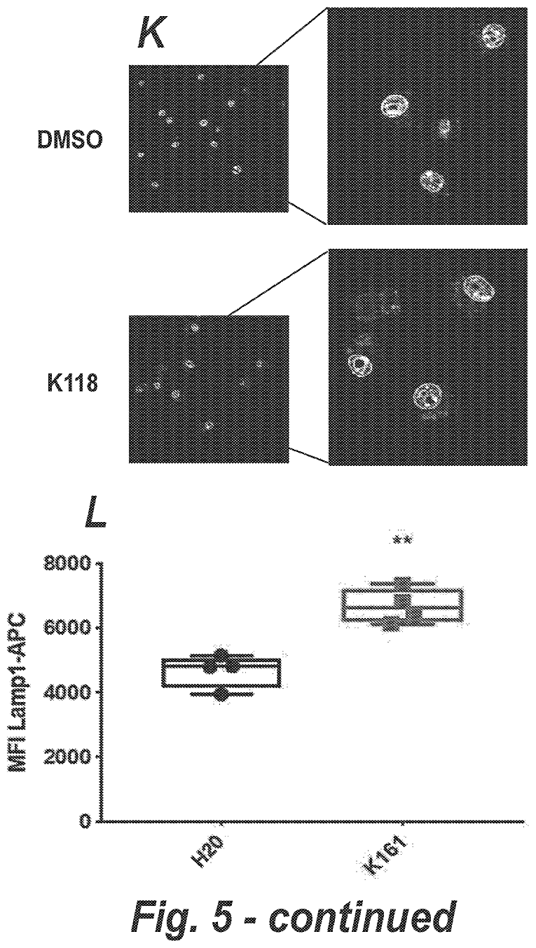

Flow cytometric assays were conducted to detect and compare the ability of SHIP inhibitor compounds to increase the phagocytic capacity of microglia as determined by LysoTracker Red staining which is preferentially retained and fluoresces in the acidic environment of the lysosomal/phagosomal subcellular compartment. BV2 cells were treated for 20 hr with 3AC, K118 or K149 at the indicated doses vs. their respective diluent controls, washed twice and incubated with 50 nM LysoTrackerRed and then analyzed by flow cytometry (FIG. 5). Histograms of the LysoTrackerRed staining for each compound at the indicated dose or their diluent control (0 μM) are shown. The compound 3AC promoted only a modest increase in phagosomal compartment size, while the two pan SHIP1/2 inhibitors K118 and K149 promoted robust increases, with K118 being more potent at all concentrations tested relative to K149. K118 repeated towards BV2 cells (FIG. 5D, 5E) and primary microglial cells (FIG. 5F, 5G). BV2 cells were treated for 16 hr with the indicated K118 concentration and incubated with LysoTrackerRed (50 nM), washed twice and then analyzed by flow cytometry and compared to similarly treated vs. their diluent control primary microglia (0.5% DMSO). Histograms of LysoTrackerRed staining for K118 (Red—3.75 μM, Green—5 μM) or diluent control (Blue, shaded) are shown. In FIG. 5E, line and whisker plots are shown for the mean fluorescence intensity (MFI) of LysoTrackerRed staining at the indicated concentrations of K118. The increases in phagocytosis observed were statistically significant. Like K118, K149 and 3AC also showed a statistically significant increase in the phagosomal compartment size at these concentrations in BV2 cells (not shown).

K118 effects were also explored towards primary microglial cells (FIG. 5F, 5G). Primary microglia were prepared from neonatal brains and treated for 16 hr with K118 at 1.5 μM and incubated with LysoTrackerRed (50 nM), washed twice and then analyzed by flow cytometry and compared to diluent control-treated primary microglia (0.5% DMSO). Histograms of LysoTrackerRed staining for K118 (Red) or diluent control (Blue, shaded) are shown in FIG. 5F, and the whisker plots for the mean fluorescence intensity (MFI) of LysoTrackerRed staining in FIG. 5G. K118 significantly increased the phagocytosis by both BV2 cells and primary microglial cells.

In a subsequent experiment, a flow cytometry assay was performed on BV2 cells (FIG. 5H, 5I) and primary microglia (FIG. 5J) treated for 16 hr with a series consisting of SHIP1 inhibitor (3AC), SHIP2 inhibitor (AS1949490), or pan SHIP1/2 inhibitors (K118, K149, K116, K161, K185), at a concentration of 3.75 μM (BV2) or 1.25 uM (primary microglial cells), or their vehicle controls, followed by incubation for 30 min with 50 nM Lysotracker Red. Pan-SHIP1/2 inhibition increased microglial lysosomal/phagosomal compartment. Confocal images of fixed BV2 cells with Lysotracker Red (red channel) and DAPI (white channel) showed an increase in lysosomal/phagosomal compartment size of K118 versus the control (FIG. 5K). FIG. 5L shows quantitation of results obtained with K161.

### Example 4: SHIP Inhibitors Increase the Phagocytic Activity of Microglia Cells Towards Dead Neurons

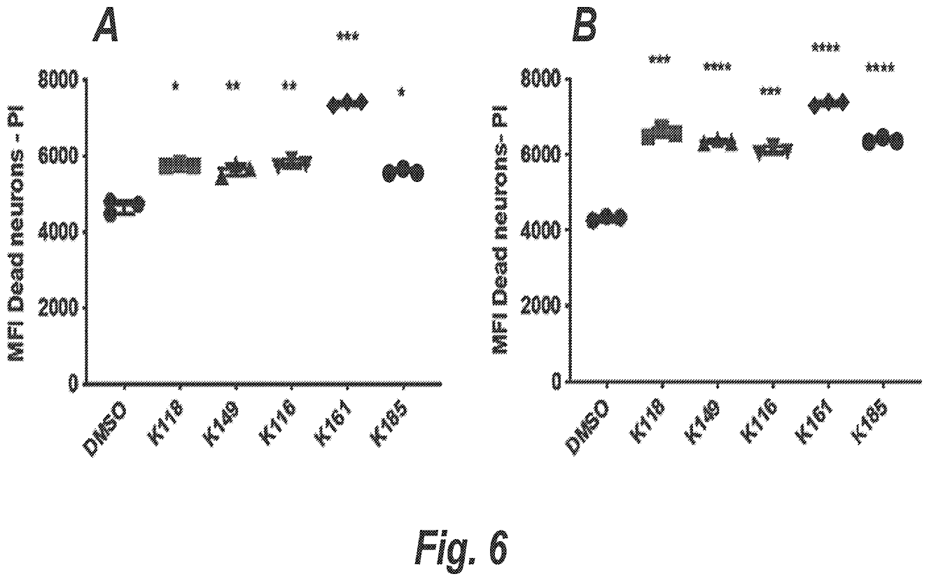

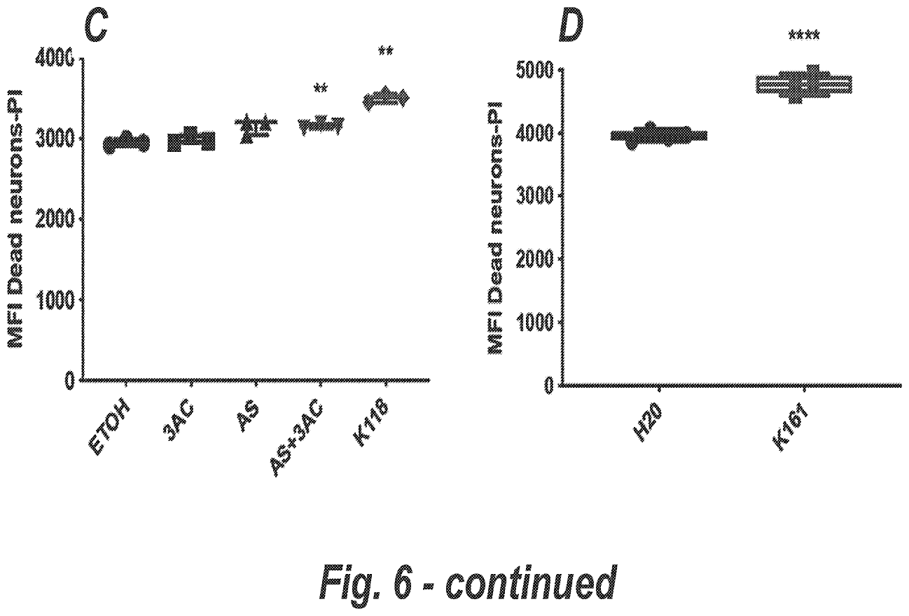

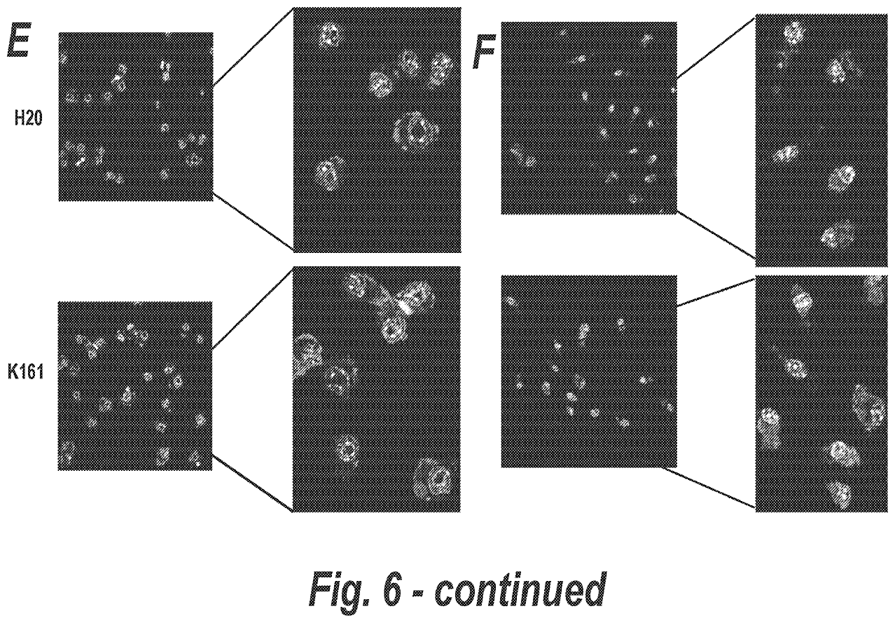

Next it was investigated if microglial can phagocytose dead neurons, and to what extent the phagocytosis of dead neurons is enhanced by SHIP inhibitors. Dead neurons were stained with propidium iodide. Propidium iodide is a fluorescent compound that binds to a cell's DNA and can be used to stain dead cells. The phagocytosis of propidium iodide (PI)-stained dead neurons by microglia was investigated by flow cytometry (FIG. 6). Sim-A9 cells (FIG. 6A), BV2 cells (FIGS. 6B and 6C) were treated for 16 hr with pan SHIP1/2 inhibitor (K118, K149, K116, K161, K185) at a concentration of 3.75 M and primary microglia (FIG. 6D) were treated with 1.25 μM of K161, or their vehicle controls, followed by incubation for 2 hours with 5×10⁴ apoptotic neurons per mL. All the pan SHIP1/2 inhibitors tested increased phagocytosis of apoptotic neurons compared to the DMSO vehicle control, in SIM-A9 cells (FIG. 6A) as well as in BV2 cells (FIG. 6B). Further, the combination of compounds AS1949490 and 3AC, but not either of those inhibitors alone, increased phagocytosis. (FIG. 6C). Also, K161 enhanced the phagocytic activity of primary microglial cells (FIG. 6D). Confocal images showed the phagocytosis of dead neurons in the absence and presence of K161 by BV2 cells in (FIG. 6E; left images) and by primary microglial cells in (FIG. 6F; right images). Pan-SHIP inhibition increased phagocytic activity of microglial cells towards dead neurons.

### Example 5: SHIP Inhibitors Increase the Phagocytic Activity of Microglia Cells Towards Amyloid Peptide

It was investigated if the enhanced phagocytic activity of microglia towards lysotracker and dead neurons extended to the enhanced phagocytosis of amyloid proteins. Flow cytometric assays were conducted to detect and compare the ability of SHIP inhibitor compounds to enhance the phagocytosis of amyloid beta 1-42 coupled to FITC. These assays provided a means to compare the ability of SHIP inhibitor compounds to promote a crucial effector function of microglia in the brain that is highly relevant for the pathology of Alzheimer's disease.

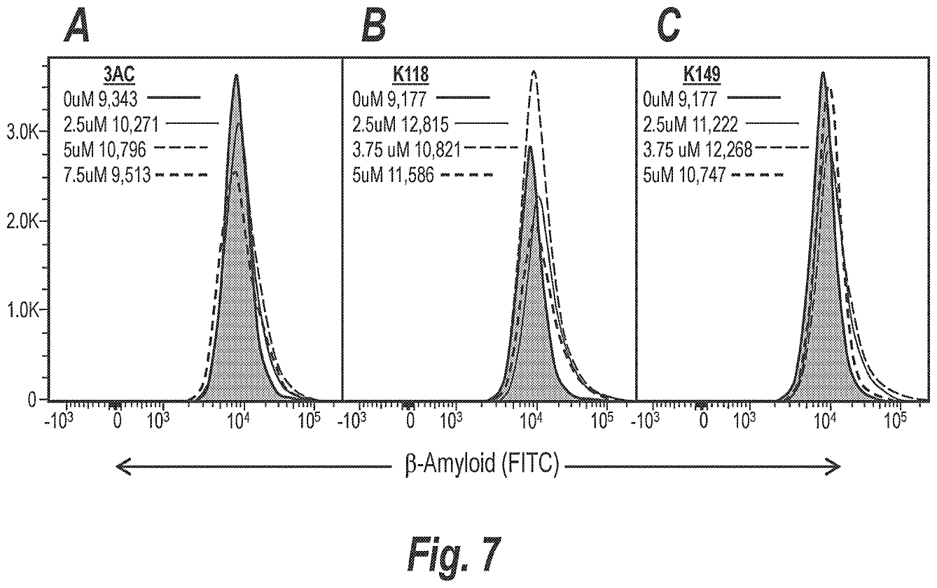

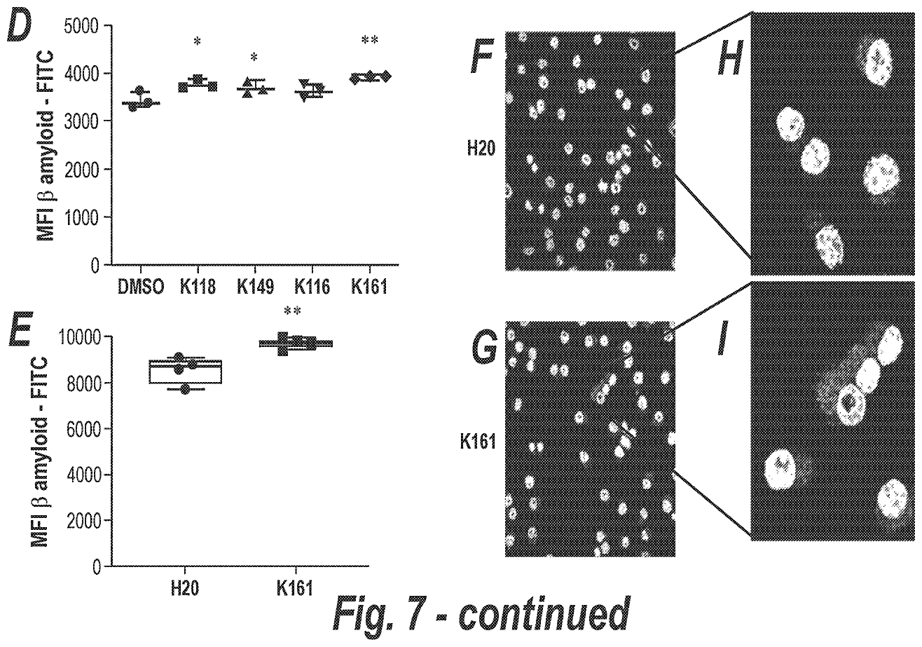

BV2 cells were treated for 20 hr with 3AC (FIG. 7A), K118 (FIG. 7B) and K149 (FIG. 7C) at the indicated doses vs. their respective diluent controls, washed twice and then incubated with FITC-labeled amyloid beta (1-42) at 0.5 μM for 3 hr. Cells were then washed twice and analyzed by flow cytometry for the presence of FITC-labeled amyloid beta (1-42). Further, BV2 cells (FIG. 7D) and primary microglia (FIG. 7E) were treated for 16 hours with one of the pan SHIP1/2 inhibitors (K118, K149, K116, or K161) at a concentration of 3.75 μM for the BV2 cells or at a concentration of 1.25 μM for the primary cells, or with their vehicle controls, followed by incubation for 1 hour with 0.5 μg/of beta amyloid coupled to FITC. Significant increases in beta amyloid phagocytosis were observed in BV2 cells treated with K118, K149 and K161 (FIG. 7D), and in microglia treated with K161 (FIG. 7E). FIG. 7F-H show the confocal images of fixed BV2 cells incubated with beta amyloid with and without K161. In cells incubated with K161 the phagosomal component is increased.

### Example 6: Assessment of the Role of mTOR in SHIP Inhibitor Effect on Microglia Cells

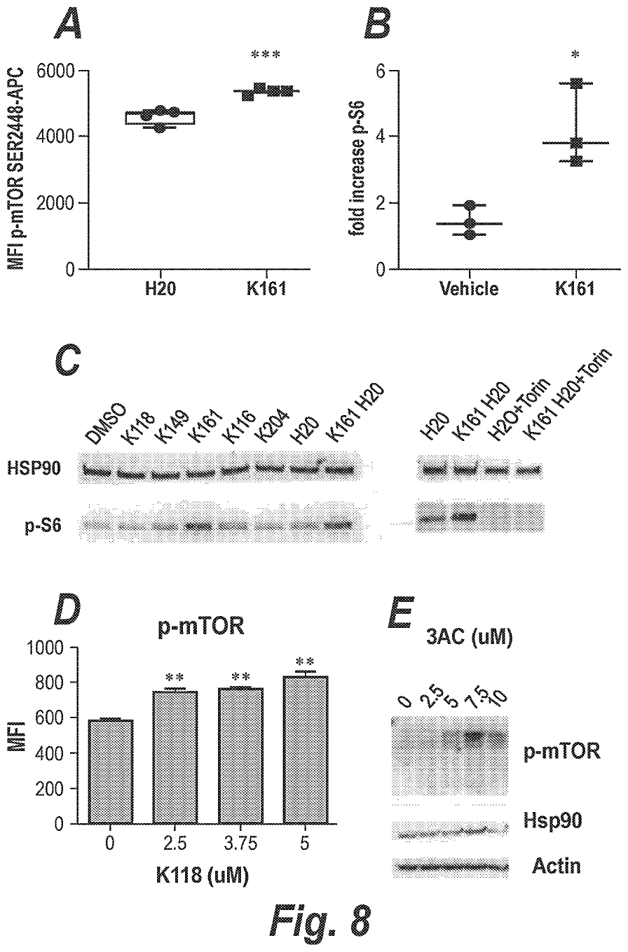

Using both Western blotting and intracellular flow (icFlow) assays activation of key signaling kinases that are known to control proliferation, survival and effector functions in immune cells were examined. This included probing for phosphorylated forms of Akt, ERK, mTOR (p-mTOR) and S6 (p-S6). BV2 cells were incubated with a series of pan-SHIP inhibitors. FIG. 8A shows the mean fluorescence intensity (MFI) for p-mTOR (Ser2448) in BV2 cells incubated for 16 hr with K161. K161 significantly increased p-MTOR expression (FIG. 8A). Likewise, K118 increased p-mTOR expression (FIG. 8D), shown as an increase in the mean fluorescence intensity observed at the indicated doses. Increases in the expression of p-S6 are shown in FIG. 8C, and the fold increase in p-S6 expression by K161 is depicted in FIG. 8B. In FIG. 8E, the effect of 3AC on BV2 cells is depicted, as assessed by Western Blotting, with actin and HSP90 as loading controls. Data suggest that mTOR is activated by SHIP inhibitors in microglia, because the SHIP1 selective inhibitor 3AC and the pan-SHIP1/2 inhibitors K118, K149 and K161 all increase mTOR phosphorylation at Ser2448—a hallmark of its activation.

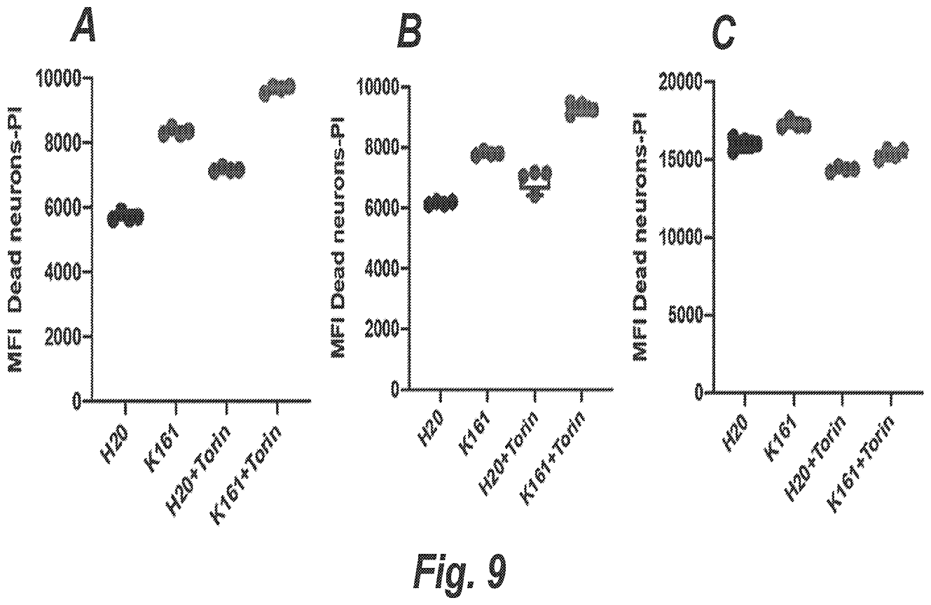

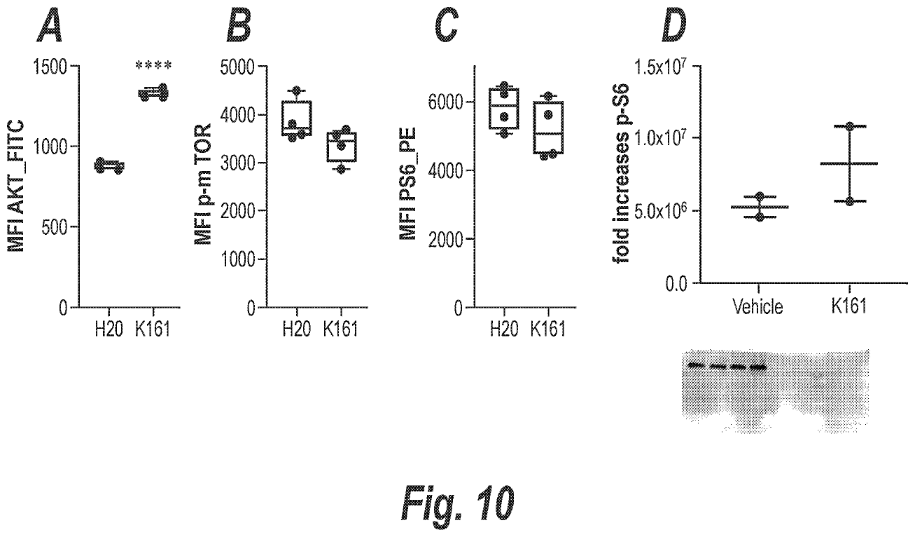

Effects of inhibitors of mTOR phosphorylation on effector functions are shown in FIG. 9. The inhibitor of mTOR phosphorylation, Torin, did not abolish the increase in phagocytosis of dead neurons by K161 in either, BV2, SIM-A9, or primary microglial cells (FIG. 9), suggesting that the effector function is independent of mTOR phosphorylation. In primary microglia cells (FIG. 10), K161 activated AKT, but not p-mTOR or p-S6.

### Example 7: In Vivo Experiments and Differentiation of the Effect of SHIP Inhibitors on Different Cell Populations

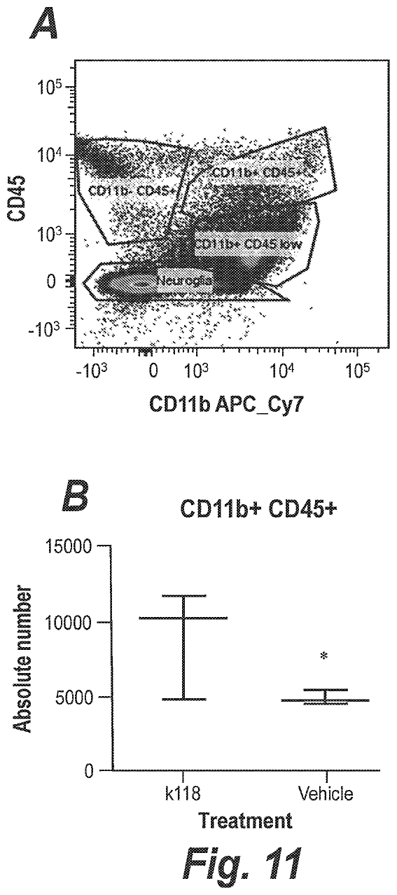

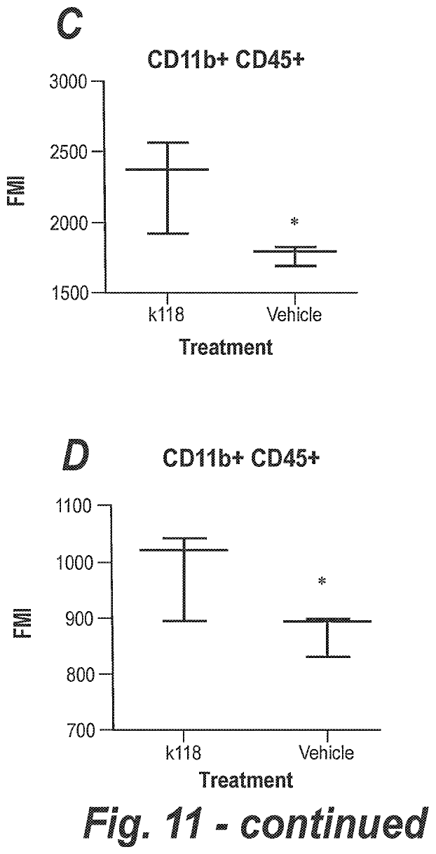

A single cell suspension of the cerebral hemispheres was prepared from K118 (10 mg/kg) and vehicle (H₂O)-treated mice (n=3/group). Mice were treated daily for two days and their brains harvested and analyzed on day 3. FIG. 11A is a representative flow cytometry plot of CD45 vs. CD11b staining in a K118-treated mouse. Line and whisker plots show the absolute number of CD11b+CD45+ cells in K118 and vehicle treated mice (FIG. 11B). The effect of treatment on the lysosomal/phagosomal compartment in the various cell populations was assessed with lysotracker red (FIGS. 11C and 11D). Line and whisker plots show the mean fluorescence intensity of LysoTracker Red staining for the CD45+CD11b− cells (FIG. 11C) and CD11b+CD45+ cells (FIG. 11D), showing that the phagocytic activity in both cell populations is increased relative to the vehicle control.

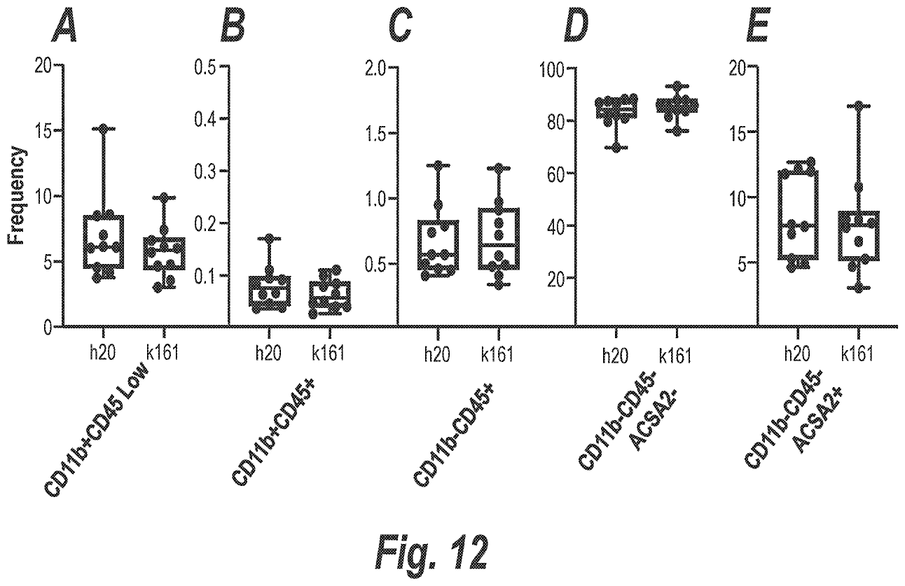

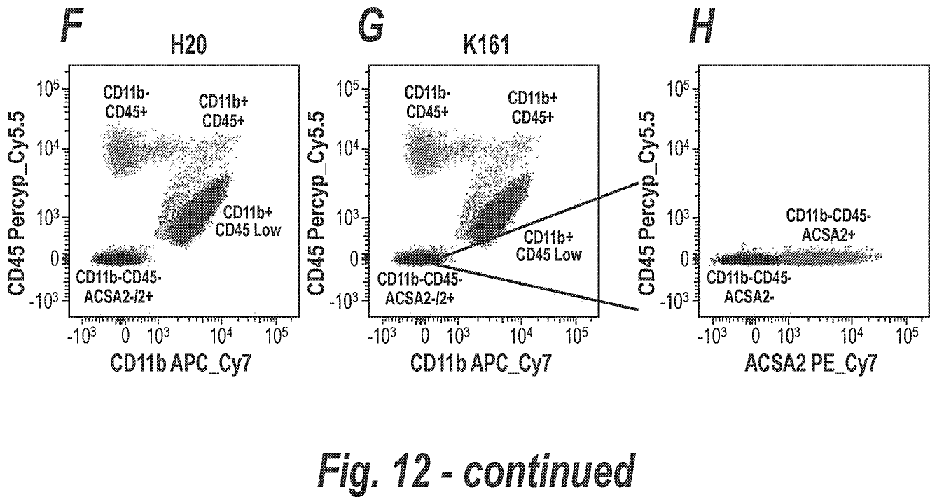

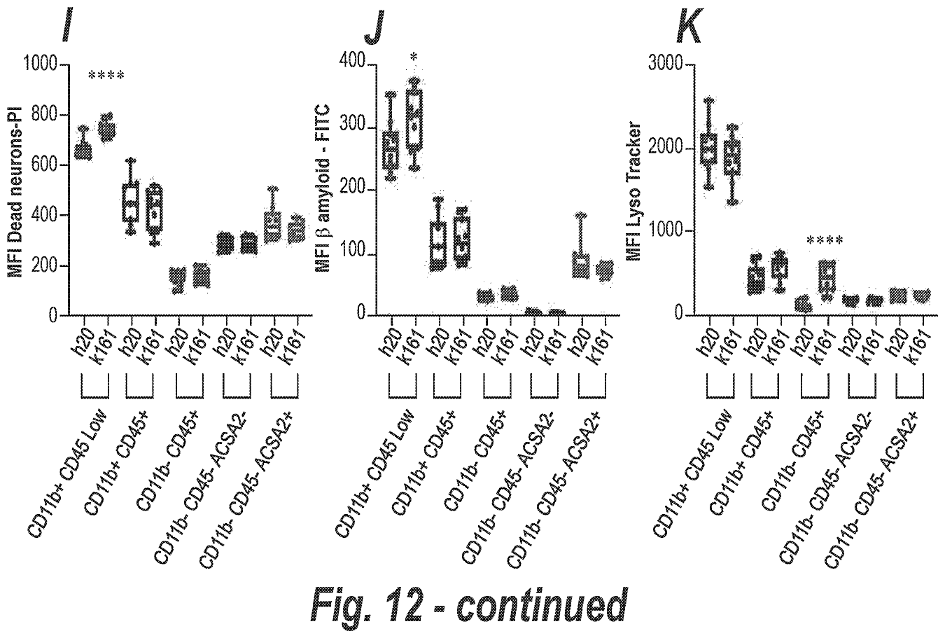

In a subsequent experiment, mice were treated twice a week for two weeks with K161. A single cell suspension of the cerebral hemispheres was prepared from K161 (10 mg/kg) and vehicle (H20)-treated mice (n=10/group). Mice were treated twice a week for two weeks, the brains harvested by flow cytometry (FIG. 12A). Box & whisker plots of frequency of single population gated on live cells singlets for markers CD11b, CD45 and ACSA2 (FIG. 12B). The ability of the various cell types to phagocytose dead neurons, beta amyloid and LysoTracker in the absence or presence of K61 are shown in FIG. 12I to 12K. A single cell suspension of the cerebral hemispheres was prepared from K161 (10 mg/kg) and vehicle (H₂O)-treated mice (n=10/group). Mice were treated twice a week for two weeks, the brains harvested by flow cytometry. Box & whisker plots of dead neuron (FIG. 12I), beta amyloid (FIG. 12J) and lysotracker assay (FIG. 12K) are shown on a single brain population gated on live cells, singlets and surface markers CD11b, CD45 and ACSA2. The results confirmed that the CD11b+CD45 low cell population, representing microglial cells, have an increased ability to phagocytose dead neurons and beta amyloid.

Overall, systemic treatment (intraperitoneal injection) of mice with a pan-SHIP1/2 inhibitory compound (K161) twice a week for two weeks increased the size of the phagosomal compartment in subsets of microglial cells present in the brains of the K161 treated mice. Therefore, SHIP inhibitors, and particularly those that inhibit both SHIP1 and SHIP2, or selectively SHIP2 (e.g., AS1949490—available from commercial suppliers such as Sigma Aldrich and Tocris), can increase microglial phagocytic capacity to remove dead neurons from the CNS. The data further shows that beta-amyloid phagocytosis by microglia is signficantly increased following incubation of cells by K118, K149, and K161.

### Example 8: Detection and Quantitation of K118 and K161 in the Brain and Serum of Mice After Single Dosing

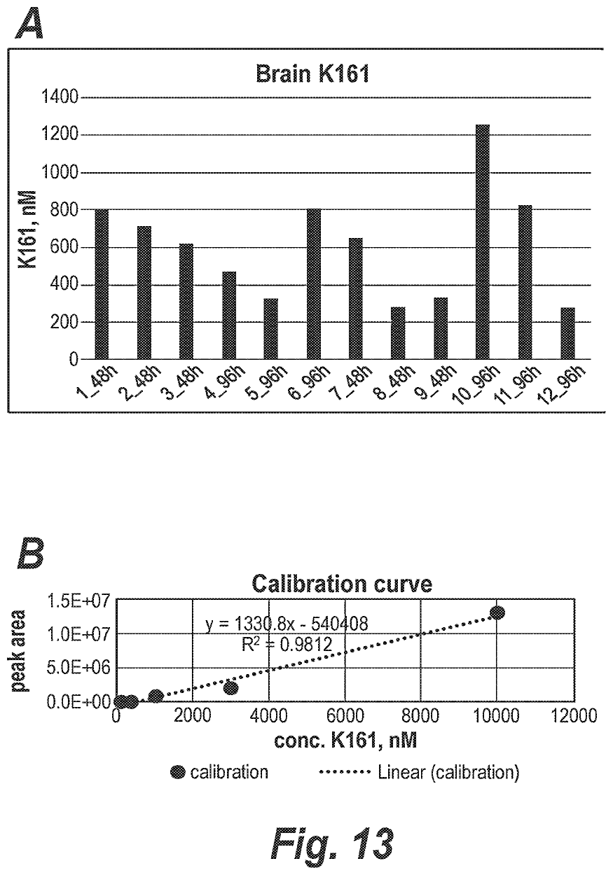

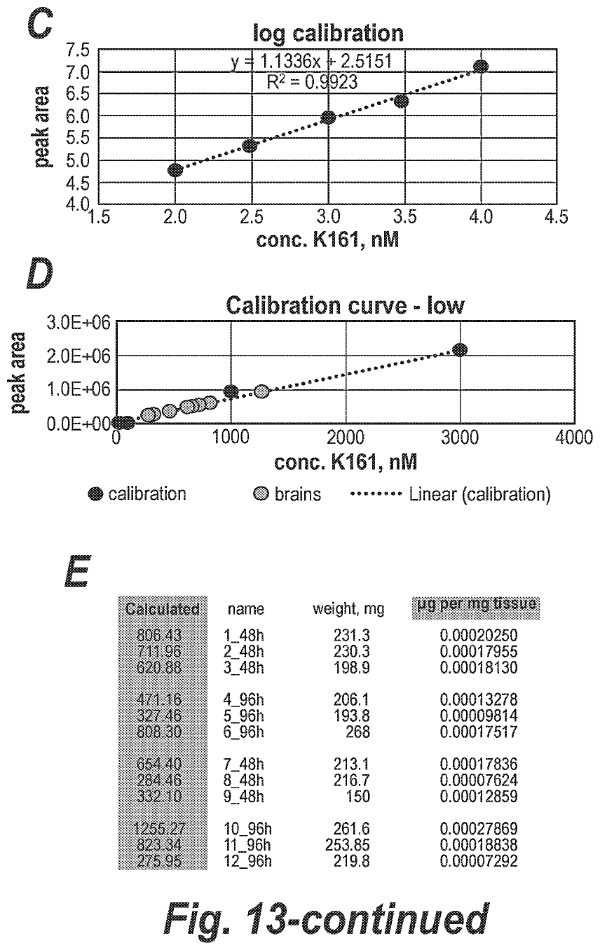

To assess if the aminosteroid pan-SHIP1/2 inhibitor can penetrate the blood brain barrier and access the CNS, K161 was dissolved in water and mice were dosed at 10 mg/kg via either intraperitoneal (IP) injection (mice 1-6) or by oral gavage (mice 7-12). Tissues were harvested 48 hr or 96 hr after administration, and K161 was detected by mass spectrometry and quantitated against a known concentration of K161 spiked into either serum or brain tissue homogenate (FIG. 13A). The calibration curves for K161 are shown in FIGS. 13B and 13C, with the added experimental data points in FIG. 13D. The concentrations of K161 in the brain tissue samples were calculated from the linear characteristics of the standard curve FIG. 13E. K161 is detectable in the brain tissue, whether K161 is administered by IP or by gavage, indicating that K161 can penetrate the blood brain barrier and access the cells in the CNS.

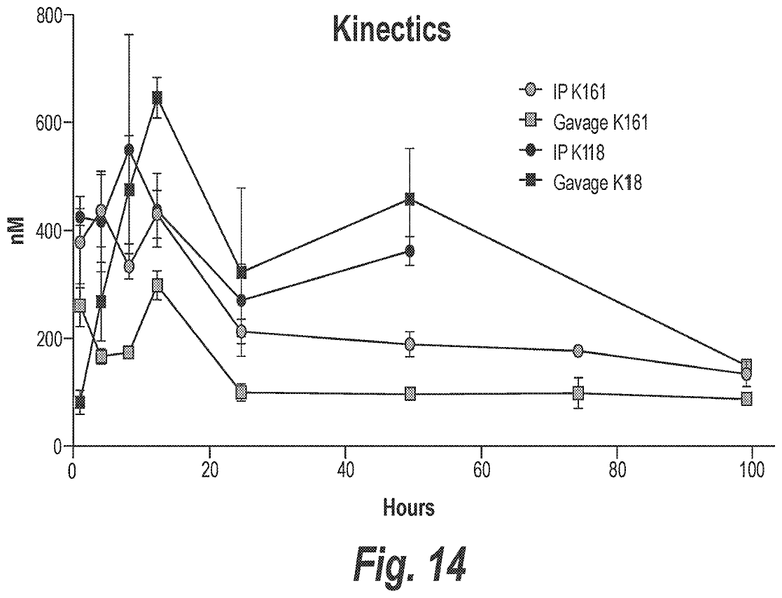

To assess oral bioavailability, K118 and K161 were administered in a single dose of 10 mg/kg either by IP or by oral gavage, and levels of each were measured in serum (FIG. 14). Both K118 and K161 have oral bioavailability, but K161 was found to decay more rapidly in vivo than K118, possibly related to differences in solubility between the two compounds.

### Example 9: Method for Preparing Compounds of Formula (III)

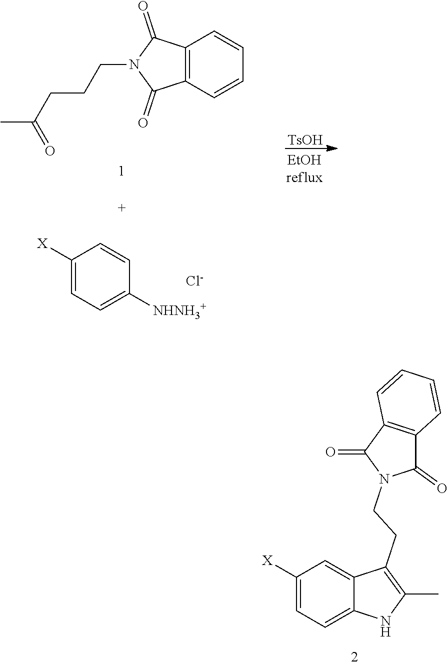

General Method for preparation of Compound 2: Corresponding phenyl hydrazine (1 eq, 1.52 mmol) and ketone 1 (0.9 eq, 1.36 mmol) were dissolved in 5 mL of ethanol. TsOH·H₂O (4 eq, 6.08 mmol) was added. The reaction mixture was heated to reflux for approximately 18 h. The reaction mixture was then cooled to room temperature and poured into 30 mL of IM NaOH. The mixture was then extracted with dichloromethane (3×20 mL). The organic extracts were dried over Na₂SO₄, filtered and concentrated to afford Compound 2. These compounds were purified using mixtures of ethyl acetate in hexanes. The stated TLC solvent system is used for purifying the indoles.

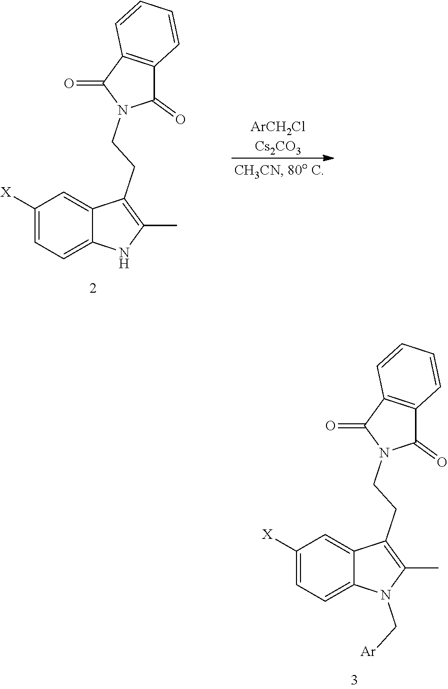

General Procedure for preparation of Compound 3: The corresponding Compound 2 (1 eq, 0.33 mmol) was dissolved in 6 mL acetonitrile. Cesium carbonate (6 eq, 1.98 mmol) was added and the mixture was heated to 80° C. The corresponding benzyl halide was then added. The reaction mixture was maintained at 80° C. for 18 h. The reaction was quenched with water (10 mL), the organic layer separated, and the aqueous layer extracted with ethyl acetate (3×10 mL). The combined organic extracts were dried with Na₂SO₄, filtered, and concentrated. Purification by silica gel chromatography afforded Compound 3. The stated TLC solvent system is used for purifying the indoles.

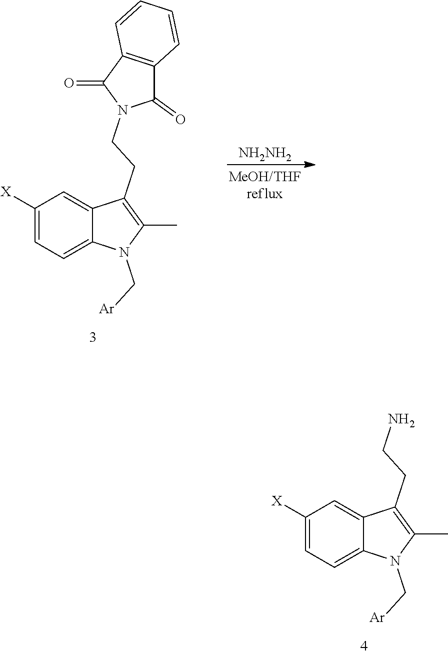

General Method for preparation of Compound 4: Compound 3 (1 eq, 0.38 mmol) was dissolved in 5 mL methanol. Hydrazine hydrate (85%, 5 eq, 1.90 mmol) was added. The reaction mixture is refluxed for 0.5 h. The reaction mixture was cooled and concentrated. The resulting residue was dissolved in dichloromethane and purified by silica gel chromatography (90% dichloromethane: 9% methanol: 1% ammonium hydroxide) to afford Compound 4.

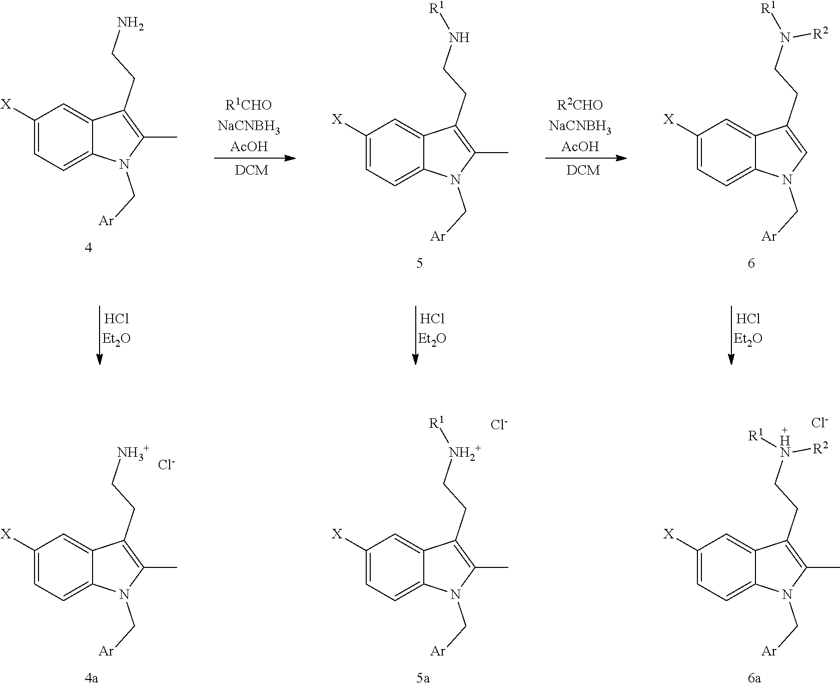

General Method for preparation of compounds 4a, 5a, and 6a: The corresponding compound (1 eq. 0.40 mmol) was dissolved in 1 mL of ether. HCl. Et₂O (10 eq, 4.0 mmol) was added. The reaction mixture was allowed to stand for 20 minutes and then concentrated. Recrystallization from mixtures of ether, hexanes or methanol afforded the corresponding HCl salt.

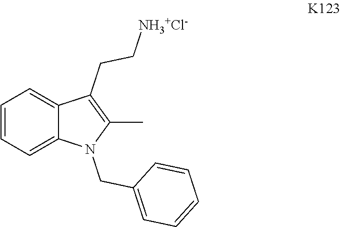

2-[1-benzyl-2-methyl-5-1H-3-yl] ethanaminium chloride. Obtained as white solid. mp=158-161° C. (50% ether in hexanes). IR (thin film) 3420, 2917, 2942, 2890, 1554, 1375, 1238 cm⁻¹; ¹H NMR (300 MHz, CDCl₃) δ 7.78 (s, 1H), 7.62 (s, 1H), 7.47 (s, 1H), 7.17 (t, J=6.0 Hz, 1H), 6.98 (d, J=6.0 Hz, 1H), 6.13 (d, J=6.0 Hz), 2.89 (bs, 4H), 2.24 (s, 3H), 2.19 (s, 3H). ¹³C NMR (75 MHz, DMSO-d₆) δ 139.1, 136.8, 134.8, 134.6, 129.2, 127.8, 127.7, 126.9, 121.3, 119.6, 118.6, 110.3, 106.7, 46.3, 33.5, 10.6.

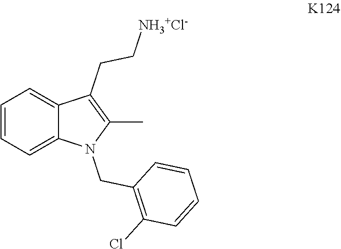

2-[1-(2-chlorobenzyl)-2-methyl-5-1H-3-yl] ethanaminium chloride. 29a. Obtained as a yellow solid. mp=161-169° C. (methanol). IR (thin film) 3466, 2988 2942, 2910, 1561, 1448, 1375, 1242, 939 cm⁻¹; ¹H NMR (300 MHz, CDCl₃) δ 7.43-7.41 (m, 1H), 7.39-7.31 (m, 1H), 7.29-7.27 (m, 2H), 7.21-7.15 (m, 2H), 7.05-7.02 (m, 1H), 6.93-6.85 (m, 1H), 5.28 (s, 2H), 3.36 (bs, 2H), 2.87-2.83 (m, 4H), 2.21 (s, 3H). ¹³C NMR (75 MHz, DMSO-d₆) δ 136.7, 136.0, 134.8, 131.8, 130.1, 128.3, 127.9, 127.5, 121.6, 119.8, 118.3, 110.0, 107.2, 44.5, 38.0, 22.7, 10.4.

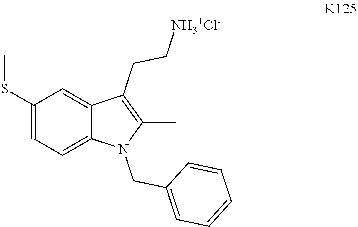

2-[1-benzyl-2-methyl-5-(methylmercapto)-1H-3-yl] ethanaminium chloride. Obtained as a white solid. mp =178-189° C. (40% ether in hexanes). IR (thin film) 3001, 2990, 1716, 1650, 1363, 1222, 1093, 760 cm⁻¹; ¹H NMR (300 MHz, CDCl₃) δ 7.73-7.72 (m, 2H), 7.62-7.59 (m, 2H), 7.56 (s, 1H), 7.43-7.40 (dd, J=6.0, 2.0 Hz, 1H), 7.33-7.28 (m, 1H), 7.24-7.21 (m, 1H), 7.14-7.10 (m, 2H) 7.02-6.95 (m, 2H), 4.72 (s, 2H) 3.94-3.83 (m, 2H), 3.09-3.04 (m, 2H), 2.54 (s, 3H) 2.32 (s, 3H). ¹³C NMR (75 MHz, DMSO-d₆) δ 138.9, 136.0, 135.8, 135.7, 135.4, 129.5, 127.7, 127.9, 127.5, 121.6, 119.8, 118.3, 110.0, 107.2, 44.5, 38.0, 22.7, 10.4.

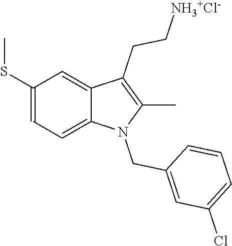

2-[1-(3-chlorobenzyl)-2-methyl-5-(methylmercapto)-1H-3-yl] ethanaminium chloride. Obtained as a black oil. TLC R_{ƒ}=0.50 (90% dichloromethane: 9% methanol: 1% ammonium hydroxide). mp=198-203° C. IR (thin film) 3430, 2910, 2790, 1543, 1375, 824 cm⁻¹; ¹³C NMR (75 MHz, DMSO-d₆) δ 9.05 (s, 3H), 7.50 (s, 1H), 7.14 (d, J=6.0 Hz, 1H), 7.29 (m, 1H), 6.93 (m, 1H), 6.13 (d, J=6.0 Hz), 3.02-2.93 (m, 4H), 2.39 (bs, 3H), 2.24 (s, 3H).

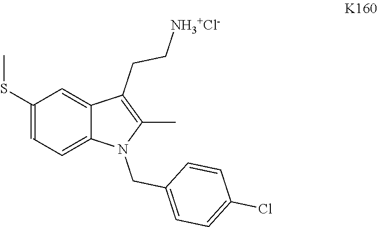

2-[1-(4-chlorobenzyl)-2-methyl-5-(methylmercapto)-1H-3-yl] ethanaminium chloride. Obtained as brown solid. mp=198-209° C. (methanol). IR (thin film) 3433, 2997, 2912, 2890, 1554, 1375, 1238 cm⁻¹; ¹H NMR (300 MHz, CDCl₃) δ 7.78 (s, 3H), 7.62 (s, 1H), 7.47 (s, 1H), 7.17 (t, J=6.0 Hz, 1H), 6.98 (d, J=6.0 Hz, 1H), 6.13 (d, J=6.0 Hz), 2.89 (bs, 4H), 2.24 (s, 3H), 2.19 (s, 3H). ¹³C NMR (75 MHz, DMSO-d₆): δ 136.4, 135.4, 134.5, 133.3, 129.2, 129.0, 127.5, 123.3, 119.3, 109.8, 108.9, 46.3, 42.0, 27.9, 19.1, 10.6.

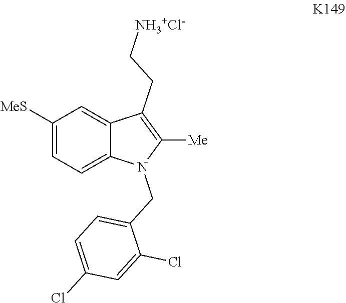

2-[1-(2,4-dichlorobenzyl)-2-methyl-5-(methylmercapto)-1H-3-yl] ethanaminium chloride. Obtained as white powder. mp=231-233° C. (methanol). ¹H NMR (400 MHz, DMSO-d₆) δ 7.84 (br s, 3H), 7.70 (d, J=2.4 Hz, 1H), 7.57 (s, 1H), 7.28-7.24 (m, 2H), 7.06 (dd, J=8.4, 1.2 Hz, 1H), 6.19 (d, J=8.4 Hz, 1H), 5.41 (s, 2H), 2.99-2.94 (m, 4H), 2.49 (s, 3H), 2.25 (s, 3H).

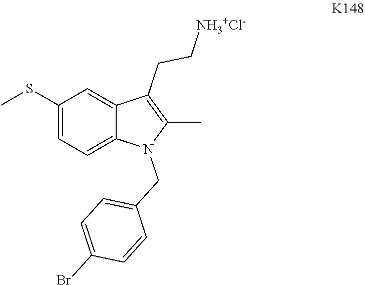

2-[1-(4-bromobenzyl)-2-methyl-5-(methylmercapto)-1H-3-yl] ethanaminium chloride. Obtained as yellow oil. IR (thin film) 3225, 2879, 2850, 1550, 1345, 1224 cm⁻¹; ¹H NMR (300 MHz, CDCl₃) δ 7.78 (s, 3H), 7.62 (s, 1H), 7.47 (s, 1H), 7.17 (t, J=6.0 Hz, 1H), 6.98 (d, J=6.0 Hz, 1H), 6.13 (d, J=6.0 Hz), 2.89 (bs, 4H), 2.24 (s, 3H), 2.19 (s, 3H). ¹³C NMR (75 MHz, DMSO-d₆): δ 135.4, 133.7, 132.6, 131.6, 131.3, 129.0, 127.9, 119.3, 111.8, 111.3, 108.0, 53.0, 40.3, 23.5, 14.1, 10.6.

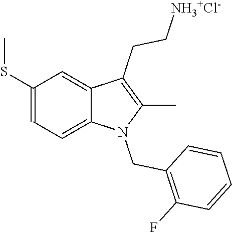

2-[1-(2-fluorobenzyl)-2-methyl-5-(methylmercapto)-1H-3-yl] ethanaminium chloride. 29g. Obtained as brown oil. IR (thin film) 3446, 2917, 2942, 2980, 1554, 1375, 1238, 764 cm⁻¹; ¹H NMR (300 MHz, CDCl₃) δ 7.50 (s, 3H), 7.47-6.96 (m, 4H), 7.86-6.79 (m, 2H), 6.31 (t, J=6.0 Hz, 1H), 5.19 (s, 2H), 2.86 (bs, 4H), 2.40 (s, 3H), 2.12 (s, 3H). ¹³C NMR (75 MHz, DMSO-d₆) δ 135.6, 135.4, 129.5, 128.4, 128.1, 127.3, 124.8, 124.3, 123.8, 123.3, 118.6, 110.0, 106.2, 105.9, 46.3, 42.0, 27.9, 19.1, 10.6.

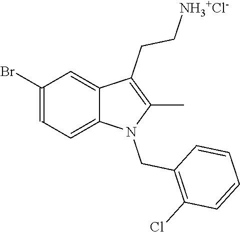

2-[1-(2-chlorobenzyl)-2-methyl-5-(bromo)-1H-3-yl] ethanaminium chloride. Obtained as pale yellow oil. IR (thin film) 3446, 2917, 2942, 2980, 1554, 1375, 1238, 764 cm⁻¹; ¹H NMR (300 MHz, CDCl₃) δ 7.60 (s, 3H), 7.29 (d, J=9.0 Hz, 1H), 7.07-7.01 (m, 2H), 6.89-6.84, 6.80 (d, J=9.0 Hz, 1H), 6.01 (d, J=9.0 Hz, 1H), 5.19 (s, 2H), 3.04 (bs, 2H), 2.96 (bs, 2H), 2.12 (s, 3H), ¹³C NMR (75 MHz, DMSO-d₆): 135.5, 135.2, 134.9, 132.0, 129.8, 129.6, 128.8, 127.5, 126.9, 128.8, 127.6, 126.8, 124.1, 120.8, 113.0, 110.7, 108.1, 44.6, 41.6, 29.9, 10.3.

### Example 10: Method for Preparing Compounds of Formula (I)

### Synthesis of 3α-Acetamido-5α-Cholestane

The 3α-Acetamido-5α-Cholestane of the present invention can be made using the following synthetic scheme:

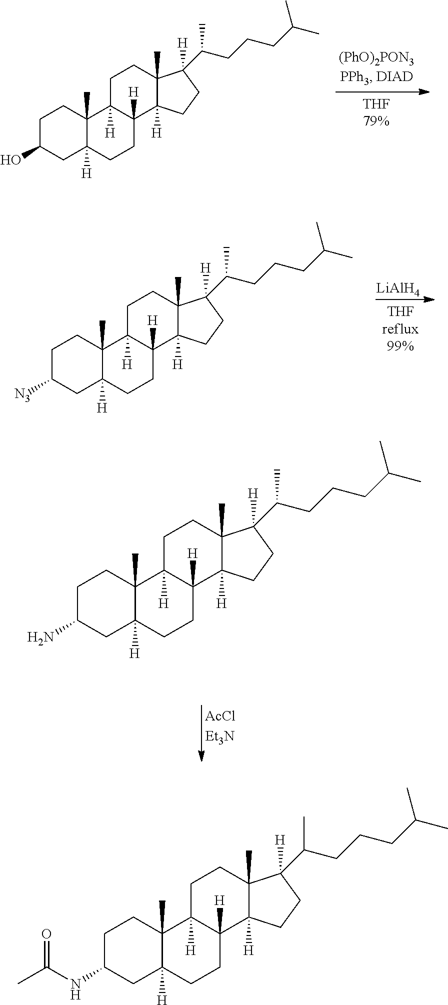

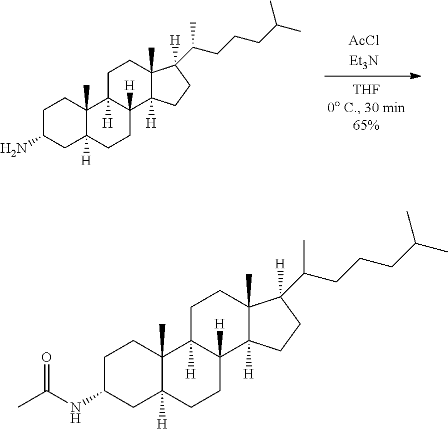

3α-Acetamido-5α-Cholestane. The α-amine (0.29 g, 0.75 mmol) was dissolved THF (2.21 mL) in a round bottom flask. Et₃N (0.12 mL, 0.90 mmol) was added dropwise and the resulting solution was cooled at 0° C. Acetyl chloride (0.06 mL, 0.83 mmol) was added dropwise into the cooled solution which resulted on the formation of white precipitate. The milky white solution was stirred continuously for 15 min at 0° C. before allowing the reaction mixture to warm up to room temperature. THF (5 mL) was added and the diluted solution was washed with HC1 (10 mL, 1 M), brine solution (10 mL), and H₂O (10 mL). The organic layer was collected, dried over Na₂SO₄, and concentrated under reduced pressure. Recrystallization of the solid residue using EtOH afforded amide (0.22 g, 65%) as off white solid.

IR (KBr): 3265, 2931, 2864, 2848, 1667, 1337 cm⁻¹. m.p.=215-216° C. ¹H NMR (300 MHz, CDCl₃): δ 5.71 (broad, 1H), 4.13 (broad, 1H), 1.99 (s, 3H), 1.96 (t, J—3 Hz, 1H), 1.79 (m 1H), 1.60-1.65 (m, 2H), 1.45-1.60 (m, 7H), 1.31-1.36 (m, 6H), 1.27-1.28 (m, 1H), 1.03-1.04 (m, 2H), 0.96-1.00 (m, 5H), 0.94-0.96 (m, 1H), 0.87 (s, J=1.2 Hz, 3H), 0.85 (d, J=1.2 Hz, 3H), 0.80 (s, 3H), 0.68-0.73 (m, 1H), 0.65 (s, 3H). ¹³C NMR (75 MHz, CDCl₃): δ 169.4, 56.7, 56.4, 54.7, 44.9, 42.7, 41.0, 40.2, 39.6, 36.3, 36.1, 35.9, 35.5, 33.3, 33.0, 32.1, 28.6, 28.4, 28.1, 26.1, 24.3, 24.0, 23.8, 23.0, 22.7, 20.9, 18.8, 12.2, 11.6

### Synthesis of the β-Amine Compound

The β-amine compound of the present invention can be made using the following synthetic scheme:

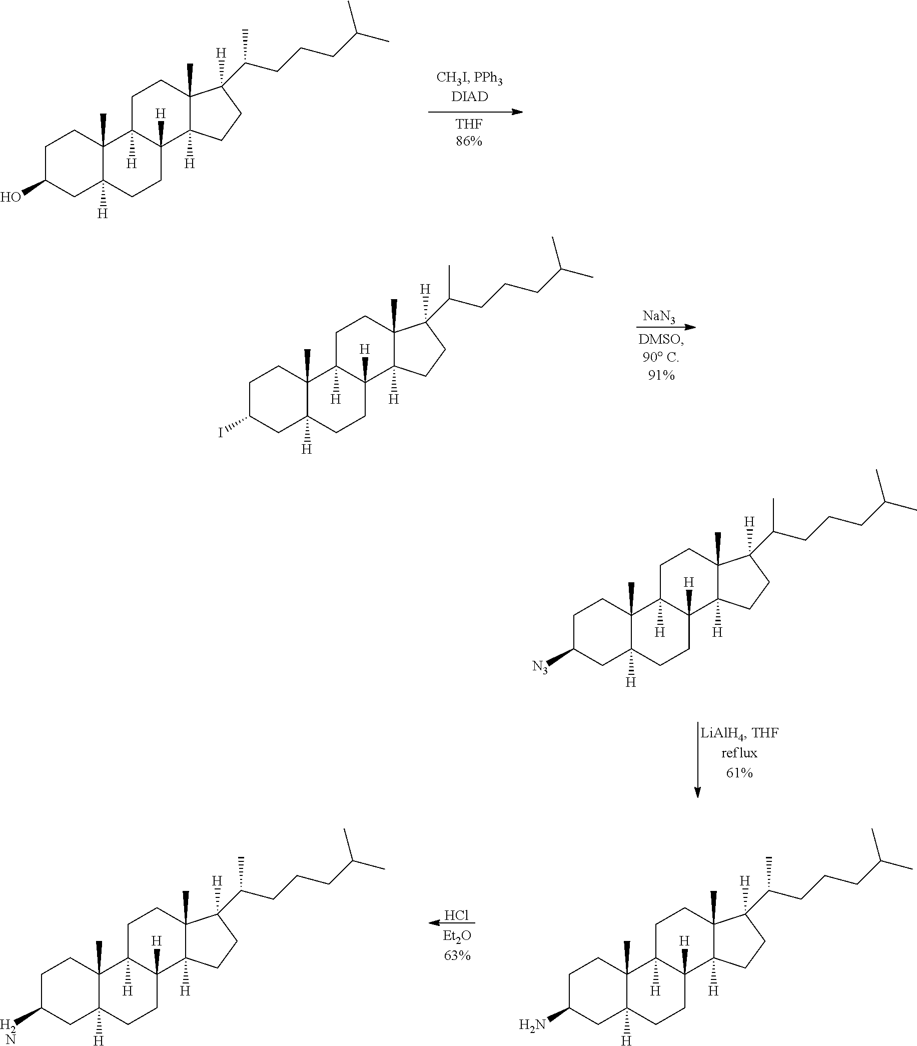

Various analogs are contemplated as being SHIP inhibitors of the present invention, as described below:

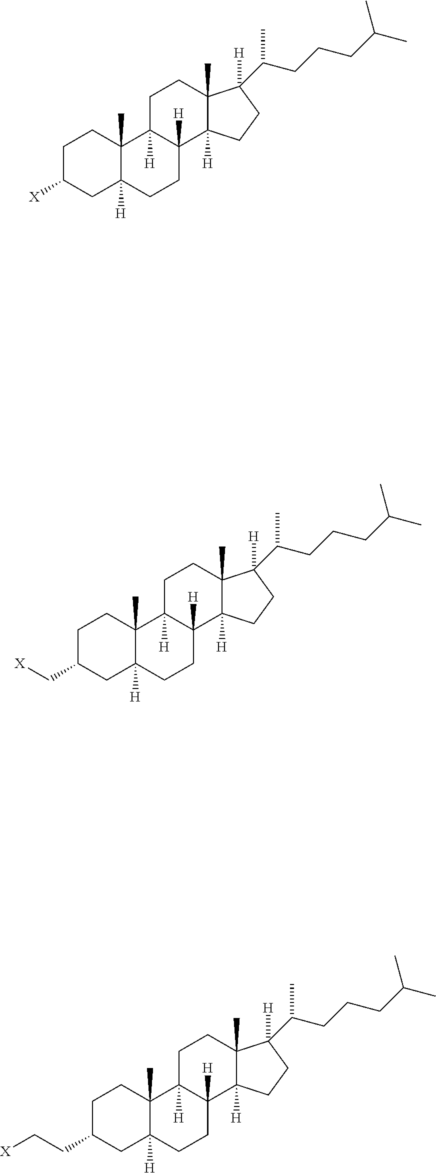

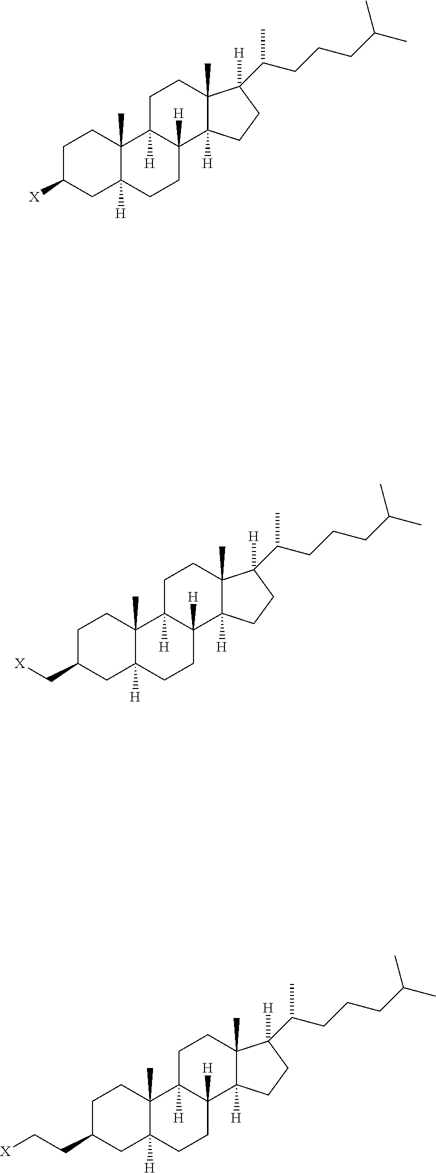

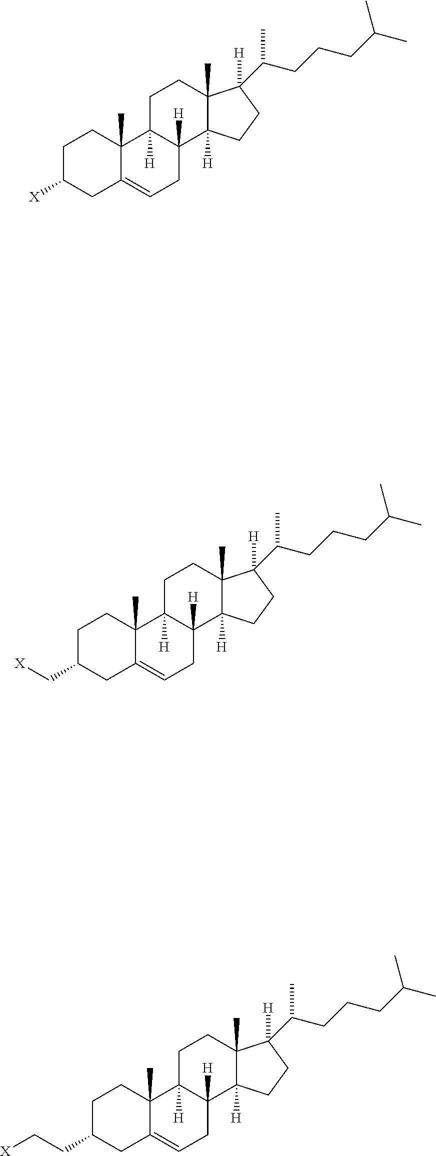

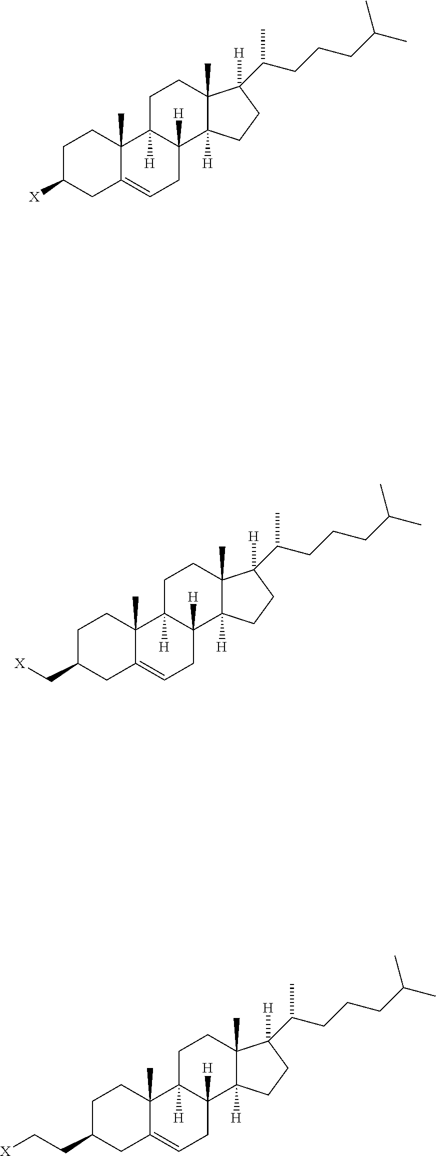

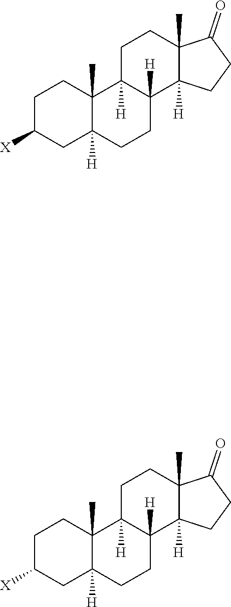

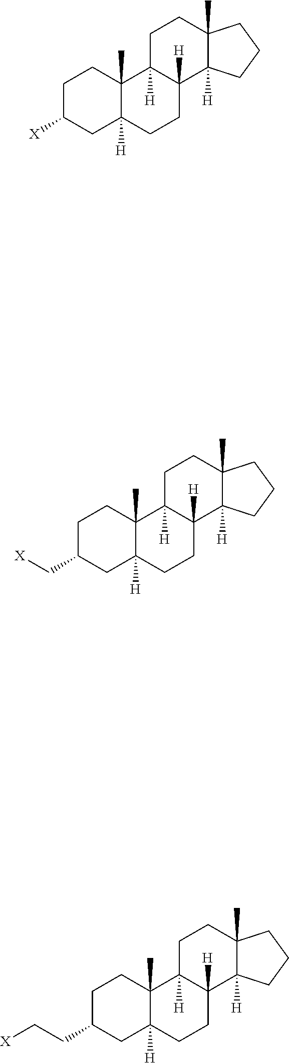

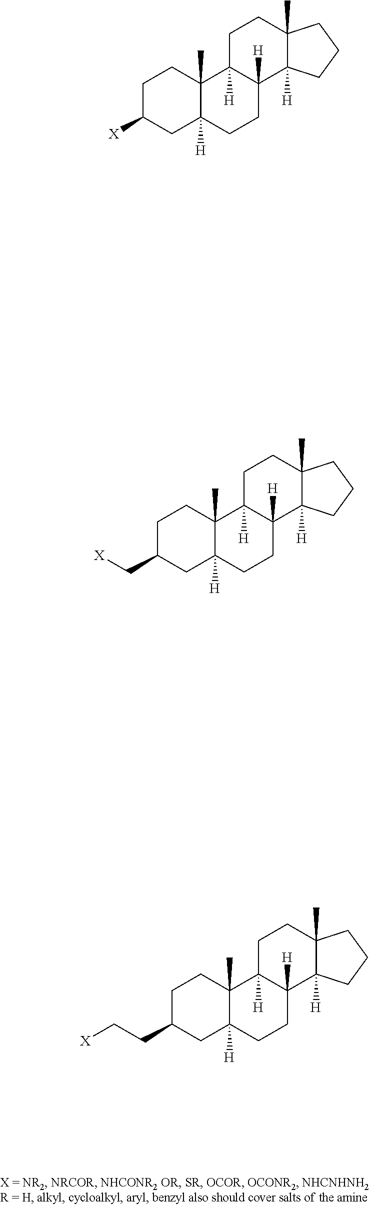

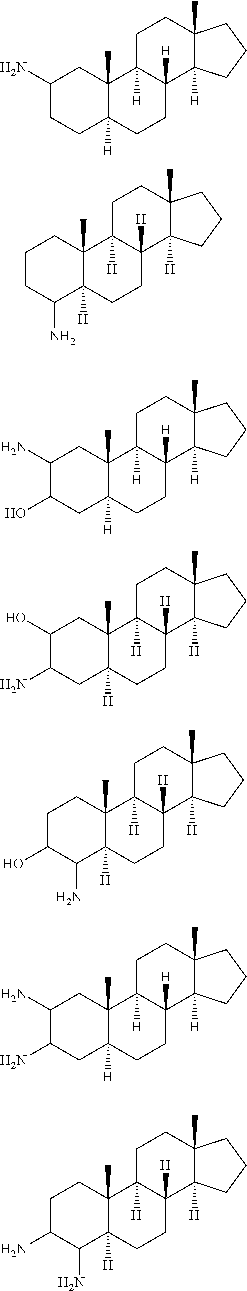

### Synthetic Schemes

Below are various schemes relating to analogs contemplated as being SHIP inhibitors of the present invention, as described below:

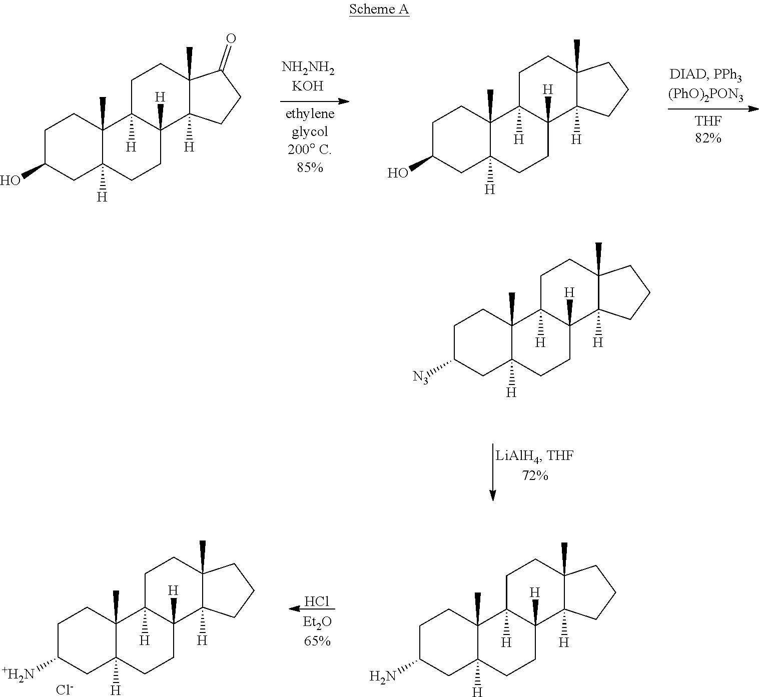

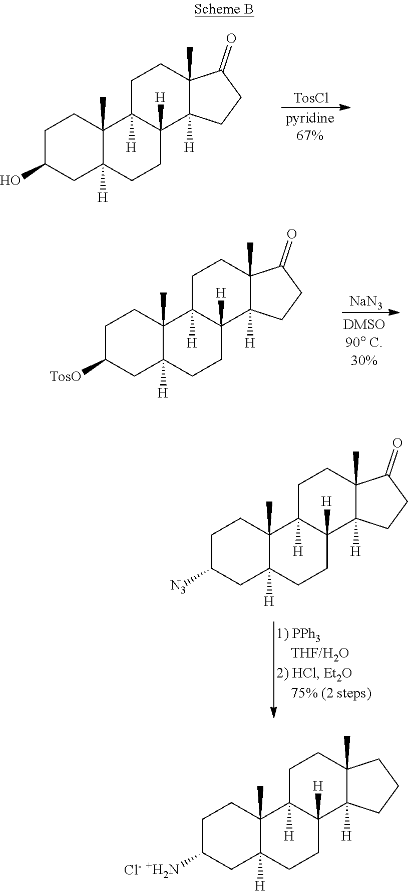

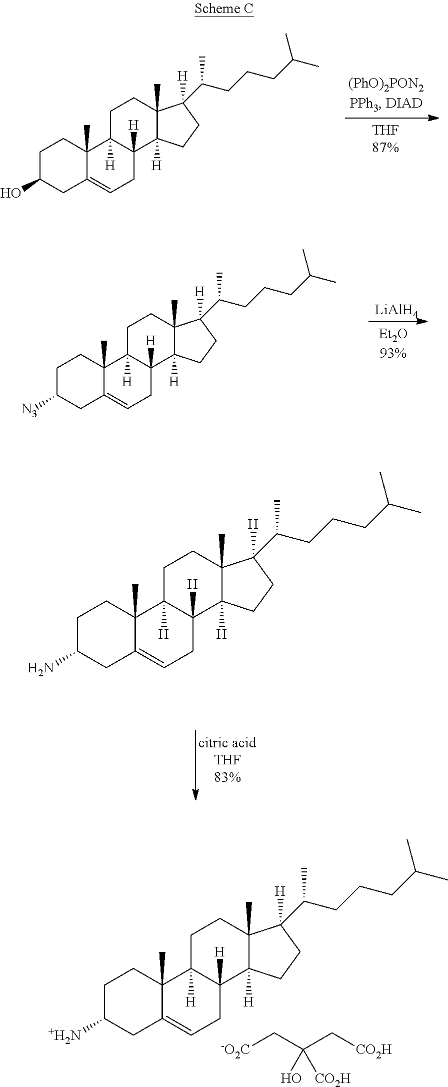

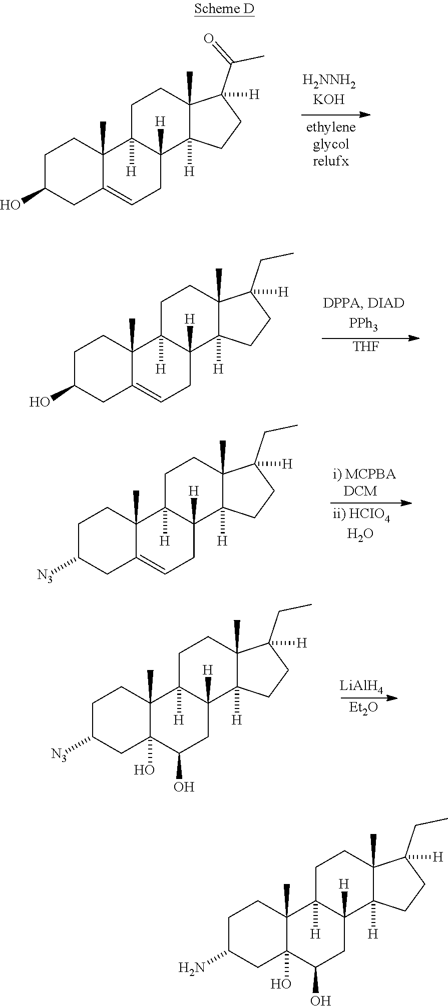

### 5α-Androstan-3β-o1

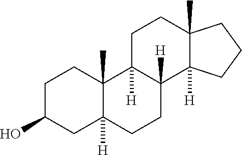

5α-Androstan-3β-o1: In a flame-dried flask, potassium hydroxide (1.58 g, 28.2 mmol) was dissolved in ethylene glycol (10 mL) by heating. The solution was cooled at room temperature before adding trans-androsterone (2.00 g, 6.89 mmol) and hydrazine hydrate (0.98 mL, 20.2 mmol). The solution was heated to reflux at 208° C. After 23 h, the solution was cooled at room temperature before adding HC1 (14.1 mL, 2M). It was extracted with CH₂Cl₂ (4×30 mL). The organic layer were collected, combined, dried over Na₂SO₄, and concentrated under reduced pressure. The resulting solid residue was recrystallized in MeOH to afford 5α-androstan-3P-ol (1.56 g, 82%). 1H NMR (300 MHz, CDC13): d 3.58 (heptet. J=4.9 Hz, 1H), 1.76-1.82 (m, 1H), 1.70-1.75 (m, 2H), 1.65-1.69 (m, 2H), 1.61-1.63 (m, 1H), 1.57-1.60 (m, 1H), 1.52-1.57 (m, 2H), 1.47-1.50 (m, 1H), 1.40-1.45 (m, 1H), 1.33-1.39 (m, 1H), 1.29-1.30 (m, 1H), 1.22-1.28 (m, 4H), 1.04-1.17 (m, 4H), 0.9-1.02 (m, 1H), 0.85-0.93 (m, 2H), 0.80 (s, 3H), 0.68 (s, 3H) 0.60-0.65 (m, 1H).

### 3α-Azido-5α-Androstane

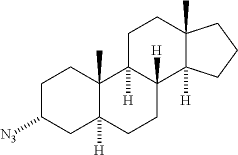

3α-Azido-5α-Androstane: In a 50 mL round bottom flask, 5α-Androstan-3β-o1 (1.12 g, 4.05 mmol) was dissolved in THF (20 mL). PPh₃ (1.06 g. 4.04 mmol) was added into the solution followed by DIAD (0.83 mL, 4.05 mmol). The resulting yellow solution was stirred continuously at room temperature for 10 min before adding (PhO)₂PON₃ (0.88 mL, 4.05 mmol). The solution was stirred continuously at room temperature. After 24 h, the reaction mixture was concentrated and the residue was recrystallized to afford 3α-Azido-5α-Androstane as a white solid (0.90 g, 74%). ¹H NMR (300 MHz, CDCl3): δ 3.88 (p, J=2.8 Hz, 1H), 1.71-1.72 (m, 1H), 1.67-1.70 (m, 3H), 1.59-1.64 (m, 2H), 1.57-1.53 (m, 3H), 1.45-1.52 (m, 3H), 1.36-1.42 (m, 2H), 1.261.31 (m, 1H), 1.18-1.24 (m, 3H), 1.14-1.17 (m, 2H), 1.13-1.10 (m, 1H), 0.85-1.03 (m, 2H), 0.79 (s, 3H), 0.72-0.77 (m, 1H), 0.69 (s, 3H).

### 3α-Amino-5α-Androstane

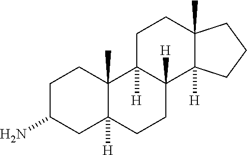

3α-Amino-5α-Androstane: In round bottom flask. LiAlH₄ (0.39 g. 9.83 mmol, 95%) was suspended in THF (10 mL). The suspension was cooled at 0° C. using ice/H₂0 bath before adding a solution of α-azide (0.90 g, 2.98 mmol) in THF (5 mL). The solution was warmed to room temperature and refluxed at 80° C. for 4 h. The reaction was cooled to room temperature before diluting the solution with THF (15 mL). The diluted reaction mixture was cooled at 0° C. and quenched using a Fieser method. The reaction mixture was stirred continuously until it turned into a milky white solution. The solution was then filtered through celite and washed with THF. The filtrate was dried over Na₂SO₄ and concentrated under reduced pressure to afford 3α-amino-5α-androstane (0.59 g, 72%). IR (KBr): 2926, 2855. 1472, 1378, 1124, 753 cm⁻¹. ¹H NMR (300 MHz, CDCl₃): d 3.18 (broad, 1H), 1.71-1.73 (m, 2H), 1.65-1.69 (m, 3H), 1.61-1.63 (m, 1H), 1.59-1.60 (m, 1H), 1.55-1.57 (m, 2H), 1.50-1.53 (m, 1H), 1.40-1.45 (m, 3H), 1.30-1.32 (m, 1H), 1.23-1.29 (m, 3H), 1.18-1.21 (m, 3H), 1.14-1.18 (m, 2H), 1.07-1.10 (m, 2H), 0.89-1.99 (m, 2H). 0.78 (s, 3H), 0.69 (s, 3H)

### 3α-Amino-5α-androstane hydrochloride

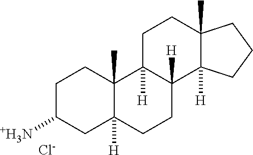

3α-Amino-5α-androstane hydrochloride: The α-amine 11 (0.20 g, 0.73 mmol) was dissolved in Et₂O (5 mL). A solution of HC1 in Et₂O (0.73 mL, 2 M) was added dropwise which resulted to the formation of precipitate. The solution was filtered and the precipitate was collected, washed over Et₂O, and dried over vacuum to afford 3α-amino-5α-androstane hydrochloride (0.15 g, 65%) as a white solid. IR (KBr): 3320, 2945, 1619, 1495, 1443, 1379 cm⁻¹. ¹F1 NMR (300 MHz, CDCl₃): d 8.45 (broad, 3H), 3.60 (broad, 1H), 1.84 (broad, 2H), 1.62-1.69 (m, 8H), 1.51-1.58 (m, 4H), 1.37-1.44 (m, 1H), 1.23-1.29 (m, 2H), 1.09-1.20 (m, 4H), 0.92-1.07 (m, 3H), 0.79 (s, 3H), 0.69 (s, 3H).

### 3α-Acetamido-5α-Androstane

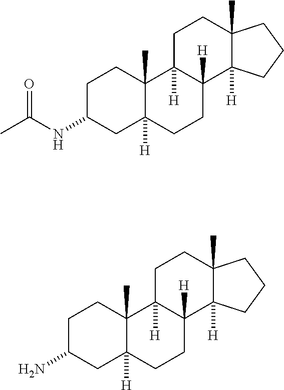

3α-Acetamido-5α-Androstane: The α-amine (0.20 g, 0.73 mmol) was dissolved THF (3 mL) in a round bottom flask. Et₃N (0.12 mL, 0.88 mmol) was added dropwise and the resulting solution was cooled at 0° C. Acetyl chloride (0.05 mL, 0.80 mmol) was added dropwise into the cooled solution which resulted on the formation of white precipitate. The milky white solution was stirred continuously for 15 min at 0° C. before allowing the reaction mixture to warm up to room temperature. THF (5 mL) was added and the diluted solution was washed with HC1 (10 mL, 1 M), brine solution (10 mL), and H₂O (10 mL). The organic layer was collected, dried over NA₂SO₄, and concentrated under reduced pressure. Recrystallization of the solid residue using EtOH afforded 3α-acetamido-5α-androstane (0.05 g, 22%) as white solid. IR (KBr): 3264, 3077, 2933, 2834. 1637,1558 cm⁻¹. ¹H NMR (300 MHz, CDCl₃): δ 5.70 (broad, 1H), 4.12 (m, 1H), 1.99 (s, 3H), 1.72-1.76 (m, 1H), 1.68-1.71 (m, 2H), 1.62-1.66 (m, 2H), 1.60-1.62 (m, 2H), 1.56-1.58 (m, 1H), 1.52-1.55 (m, 1H), 1.48-1.51 (m, 1H), 1.42-1.46 (m, 1H), 1.36-1.39 (m, 1H), 1.29-1.34 (m, 2H), 1.23-1.27 (m, 1H), 1.21 (d, J=3.0 Hz, 1H), 1.18-1.19 (m, 1H), 1.12-1.17 (m, 2H), 1.081.11 (m, 1H), 1.00-1.06 (m, 1H), 0.92-0.97 (m, 1H), 0.84-0.90 (m, 1H), 0.81 (s, 3H), 0.71-0.77 (m, 1H), 0.69 (s, 3H).

### 3β-Tosyloxy-5α-Androstan-17-one

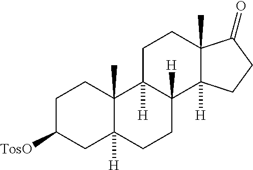

3β-Tosyloxy-5α-Androstan-17-one: In a 25 mL round bottom flask, trans-androsterone (1.00 g, 3.44 mmol) and p-toluenesulfonyl chloride (1.51 g, 7.91 mmol) was dissolved in in pyridine (4.30 mL). The reaction mixture was stirred continuously at room temperature. After 24 h, the reaction mixture was quenched by adding H2O (10 mL) and it was extracted with CH2C12 (3×20 mL). All organic layers were collected, combined together and washed over HC1 (3×20 mL, 2 M), brine solution (3×20 mL), and H2O (3×20 mL), dried over NA₂SO₄, and concentrated under reduced pressure afforded 313-tosyloxy-5a-androstan-17-one (1.33 g, 87%) as a white solid. 1H NMR (300 MHz, CDC13): d 7.79 (dt, J=8.3, 1.9 Hz, 2H), 7.33 (dd, J=8.0, 0.5 Hz, 2H), 4.42 (h, J=5.9 Hz, 1H), 2.44 (s, 3H), 2.38-2.47 (m, 1H), 1.99-2.11 (m, 1H), 1.86-1.95 (m, 1H), 1.78-1.80 (m, 1H), 1.71-1.77 (m, 2H), 1.65-1.69 (m, 1H), 1.56-1.64 (m,3H), 1.44-1.55 (m, 3H), 1.30-1.31 (m, 1H), 1.28-1.29 (m, 2H), 1.22-1.24 (m, 1H), 1.18-1.20 (m, 1H), 1.04-1.16 (m, 1H), 0.85-1.00 (m, 2H), 0.84 (s, 3H), 0.80 (s, 1H), 0.60-0.69 (m, 1H).

### 3α-Azido-5α-Androstan-17-one

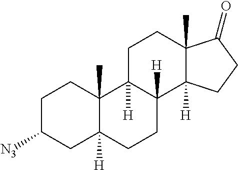

3α-Azido-5α-Androstan-17-one: A suspension of tosylate (1.33 g, 2.99 mmol) and NaN₃ (1.94 g, 29.9 mmol) in DMSO (75 mL) was heated to reflux at 90° C. After approximately 5 h, the reaction mixture was cooled at room temperature before adding H₂O (10 mL). The diluted solution was extracted with Et₂O (3×20 mL). All organic layers were collected, dried over MgSO₄, and concentrated under reduced pressure. The solid residue was recrystallized in EtOH to afford 3α-azido-5α-androstan-17-one (0.28 g, 30%). ¹H NMR (300 MHz, CDCl₃): d 3.88 (pentet, J=2.6 Hz, 1H), 2.43 (dd, J=10.3, 9.6 Hz, 1H), 2.00-2.12 (m, 1H), 1.88-1.97 (m, 1H), 1.81-1.83 (m, 1H), 1.76-1.78 (m, 1H), 1.66-1.72 (m, 2H), 1.62-1.65 (m, 1H), 1.51-1.56 (m, 2H), 1.39-1.49 (m, 4H), 1.17-1.34 (m, 7H), 0.94-1.08 (m, 1H), 0.85 (s, 3H), 0.81 (s, 3H).

### 3α-Amino-5α-androstan-17-one hydrochloride

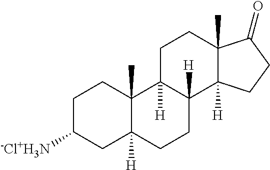

3α-Amino-5α-androstan-17-one hydrochloride: In a flame dried flask, azide (0.28 g, 0.89 mmol) and PPh₃ (0.36 g. 1.37 mmol) was dissolved in THF (15 mL). The solution was stirred continuously at room temperature for 18 h. H₂O (3 mL) was added and the solution was heated to reflux at 80° C. After 1 h, the solution was cooled at room temperature. The organic layer was collected, dried over NA₂SO₄, and concentrated under reduced pressure. The residue was dissolved Et₂O (7 mL) and a solution of HC1 (0.89 mL, 2 M) was added which resulted to formation of precipitate. The precipitate was filtered over filter paper, washed over Et2O, and dried to afford 3α-amino-5α-androstan-17-one hydrochloride (0.22 g, 76%) as white solid. IR (KBr): 3326, 2923, 1737, 1496, 1455, 731 cm⁻¹. ¹H NMR (300 MHz, CDCl₃): δ 8.42 (broad, 3H), 3.61 (broad, 1H), 2.42 (dd, J=11.1, 8.7 Hz, 1H), 2.00-2.13 (m, 1H), 1.86-1.94 (m, 2H), 1.76-1.83 (m, 3H), 1.44-1.64 (m, 7H), 1.19-1.38 (m, 6H), 0.95-1.13 (m, 2H), 0.84 (s, 3H), 0.81 (s, 3H).

### 3α-Azidocholest-5-ene

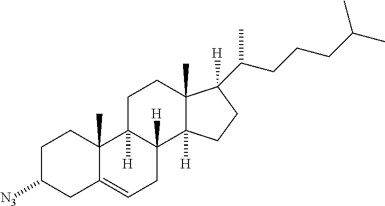

3α-Azidocholest-5-ene: Cholesterol (7.76 mmol, 3.0 g) and triphenylphosphine (7.76 mmol, 2.04 g) were dissolved in 77.6 mL of anhydrous tetrahydrofuran. Diisopropyl azodicarboxylate (7.76 mmol, 1.5 mL) was then added dropwise. After stirring the orange mixture for a few minutes, diphenylphosphoryl azide (7.76 mmol, 1.68 mL) was added dropwise. After 24 hours, the pale yellow reaction mixture was concentrated. Purification by silica gel chromatography (100% hexanes) afforded 3α-azidocholest-5-ene (2.14 g, 67%) as a white solid. mp 110-112° C.; TLC R_{ƒ}=0.87 (20% ethyl acetate/hexanes); IR (thin film) 2946, 2914, 2845, 2083 cm⁻¹; ¹H NMR (300 MHz, CDCl₃) δ 5.42-5.40 (m, 1H), 3.89 (t, 1H, J=2.9 Hz), 2.58-2.49 (m, 1H), 2.23-2.16 (m, 1H), 2.16-1.93 (m, 2H), 1.89-1.05 (m, 24H), 1.02 (s, 3H), 0.93 (d, 3H, J=6.5 Hz), 0.88 (d, 6H, J=6.6 Hz), 0.69 (s, 3H).

### 3α-Aminocholest-5-ene

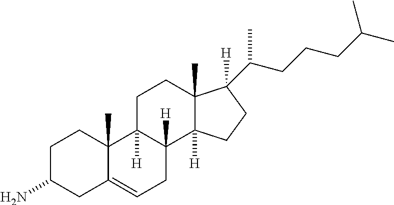

3α-Aminocholest-5-ene: 3α-Azidocholest-5-ene (4.62 mmol, 1.9 g) was dissolved in 154 mL of anhydrous diethyl ether. Lithium aluminum hydride (46.2 mmol, 1.75 g) was then added in one portion. After 30 hours, the reaction mixture was cooled to 0° C. 1.75 mL of deionized water was then added dropwise. After stirring for five minutes, 1.75 mL of 15% aqueous NaOH was added dropwise. After stirring for another five minutes, 5.25 mL of deionized water was added dropwise. The reaction was then stirred until all the salts turned white. Immediately afterwards, the reaction was filtered, dried (NA₂SO₄), and concentrated to afford 3α-Aminocholest-5-ene (1.57 g, 93%) as a white solid. mp 104-106° C.; IR (thin film) 3367, 3343, 2931, 1557 cm⁻¹: ¹H NMR (300 MHz, CDCl₃) d 5.37-5.34 (m), 1H), 3.15 (t, 1 H, J=3.2 Hz), 2.61-2.54 (m, 1H), 2.04-1.74 (m, 6H), 1.63-1.03 (m, 21H), 1.00 (s, 3H), 0.91 (d, 3H, J=6.5 Hz), 0.86 (d, 6H, J=6.6 Hz), 0.67 (s, 3H).

### 3α-Aminocholest-5-ene Hydrochloride

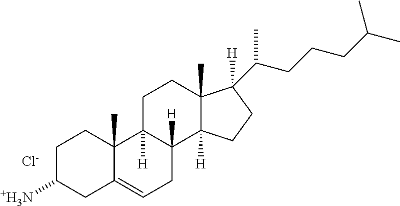

3α-Aminocholest-5-ene Hydrochloride: 3α-Aminocholest-5-ene (1.61 mmol, 0.59 g) was dissolved in 2 mL of anhydrous diethyl ether. Hydrogen chloride (2.0 M in diethyl ether) (3.22 mmol, 1.61 mL) was then added. After 3 hours, a white precipitate formed. The reaction was then filtered and the solid was washed with diethyl ether to afford 3α-aminocholest-5-ene hydrochloride (0.31 g, 46%) as a white solid. mp 293-295° C.; IR (thin film) 2947 cm⁻¹l ¹H NMR (300 MHz, CDCl₃) d 8.25 (s, 3H), 5.52 (d, 1H, J=4.3 Hz), 3.58 (s, 1 H), 2.61 (d, 1H, J=14.6 Hz). 2.36 (d, 1H, J=15.0 Hz), 2.02-1.07 (m, 26H), 1.01 (s, 3H), 0.91 (d, 3H, J=6.3 Hz), 0.86 (d, 6H, J=6.6 Hz), 0.67 (s, 3H).

### 3α-Aminocholest-5-ene Citrate

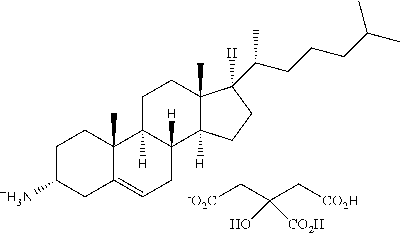

3α-Aminocholest-5-ene Citrate: 3α-Aminocholest-5-ene (0.82 mmol, 300 mg) was dissolved in 1.64 ml of tetrahydrofuran. Citric acid (0.82 mmol, 158 mg) was dissolved in 0.82 ml of tetrahydrofuran. The solution of citric acid was added dropwise to the solution of cholesterol amine. The mixture was stirred until the solution became very cloudy (approximately 15 minutes). The solution was vacuum filtered. The resulting white solid was washed with tetrahydofuran, collected, and dried under high vacuum for 12 hours to produce 298 mg of the 3α-aminocholest-5-ene citrate in 63% yield. mp: 172-174° C.; IR (thin film): 3469, 2954, 2247, 1714, 1591 cm⁻¹; ¹H NMR (300 MHz, CD₃OD) 6: 5.53 (d, 1H, J=5.2 Hz), 3.55 (s, 1H), 2.84-2.70 (m, 4H), 2.80-2.70 (m, 1H), 2.19-1.0 (m, 28H), 1.07 (s, 3H), 0.95 (3H, J=6.5 Hz), 0.89 (d, 6H, J=6.6 Hz), 0.73 (s, 3H).

### (3β)-Pregn-5-en-3-ol

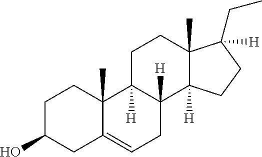

### (3β)-Pregn-5-en-3-ol

Pregnenolone (4.0 g, 12.6 mmol) was dissolved in 63 mL of ethylene glycol. Hydrazine hydrate (6.3 mL, 202 mmol) was slowly added dropwise. The reaction was refluxed for 4 h. Thin layer chromatography (20% ethyl acetate/80% hexanes) was performed to confirm that all the pregnenolone had reacted. Subsequently, the reaction was cooled to 80° C. Potassium hydroxide (9.93 g, 177 mmol) was added in three portions. The reaction was refluxed again for 24 h and then cooled to room temperature. Excess potassium hydroxide was quenched with 3 M HCl (aq) (60 mL). The aqueous layer was extracted with dichloromethane (11×60 mL). The combined organic layers were dried with MgSO₄, filtered, and concentrated in vacuo. Purification by silica gel chromatography (20% ethyl acetate/80% hexanes) provided alcohol (3β)-Pregn-5-en-3-ol (3.6 g, 94%) as a white solid.

(3β)-Pregn-5-en-3-ol. mp 126.5-128.2° C. (chloroform); TLC R_{ƒ}=0.29 (20% ethyl acetate/80% hexanes); IR (CHCl₃) 3497, 2942, 1638 cm⁻¹; ¹H NMR (300 MHz, CDCl₃) □□15.37-5.35 (m, 1H), 3.57-3.48 (m, 1H), 2.32-2.19 (m, 2H), 2.05-1.97 (m, 1H), 1.88-1.83 (m, 3H), 1.76 (dt, 1H, J=12.4, 3.0 Hz), 1.67-1.37 (m, 11H), 1.28-0.93 (m, 7H), 1.02 (s, 3H), 0.88 (t, 3H, J=7.3 Hz), 0.58 (s, 3H).

### (3α)-3-Azido-pregn-5-ene

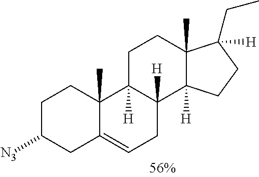

### (3α)-3-Azido-pregn-5-ene

(3α)-Pregn-5-en-3-ol (524 mg, 1.73 mmol) and triphenylphosphine (681 mg, 2.60 mmol) were dissolved in 40 mL of tetrahydrofuran. Diisopropyl azodicarboxylate (0.34 mL, 1.73 mmol) was then slowly added dropwise. After stirring the resulting orange mixture for 5 minutes, diphenylphosphoryl azide (0.37 mL, 1.73 mmol) was added dropwise. As the azide was added, the reaction gradually changed in color from orange to golden yellow. After stirring for 24 h at room temperature, the reaction was concentrated in vacuo. Purification by silica gel chromatography (2% ethyl acetate/98% hexanes) afforded (3□)-3-azido-pregn-5-ene. (318 mg, 56%) as a white solid.

(3α)-3-Azido-pregn-5-ene. mp 121.7-122.5° C. (chloroform); TLC R_{ƒ}=0.53 (3% ethyl acetate/97% hexanes); IR (CHCl₃) 2943, 2111, 2082, 1651 cm⁻¹; ¹H NMR (300 MHz, CDCl₃) 5.40-5.39 (m, 1H), 3.87 (t, 1H, J==2.9 Hz), 2.55-2.48 (m, 1H), 2.18 (dt, 1H, J=15.0, 2.8 Hz), 2.03-1.93 (m, 1H), 1.87-1.60 (m, 6H), 1.56 (s, 3H), 1.52-1.33 (m, 4H), 1.24-1.02 (m, 6H), 1.00 (s, 3H), 0.87 (t, 3H, J=7.2 Hz), 0.57 (s, 3H).

### (3α)-3-Azido-pregn-5-ene

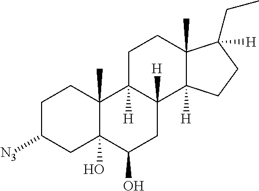

### (3α)-3-Azido-pregn-(5β,6α)-diol

(3α)-3-Azido-pregn-5-ene (1.61 mg, 4.92 mmol) was dissolved in 70 mL of dichloromethane and cooled to 0° C. Meta-chloroperoxybenzoic acid (1.7 g, 9.84 mmol) was then added in a single portion. The reaction was stirred for 24 h at room temperature. Thin layer chromatography was performed to confirm that all of azide had reacted. 10% Na₂CO₃ (aq) (10 mL.) was then added. After stirring for 15 minutes, the reaction was re-cooled to 0° C. and then acetone (25 mL) was slowly added. An aqueous solution of perchloric acid was prepared by diluting 70% HClO₄ (aq) (10 mL) with water (20 mL). The prepared HClO₄ (aq) solution was then slowly added dropwise and the reaction was warmed to room temperature over a period of 24 h. The reaction was then diluted with ethyl acetate (80 mL) and washed with brine (3×160 mL), 10% Na₂CO₃ (aq) (3×160 mL), water (2×160 mL), and once more with brine (160 mL). The combined organic layers were dried with MgSO₄, filtered, and concentrated in vacuo to give a dark brown oil. Purification by silica gel chromatography (12% ethyl acetate/84% hexanes) afforded (3α)-3-azido-pregn-(5β,6α)-diol (1.06 g, 60%) as a white solid. (3α)-3-Azido-pregn-(5β,6α)-diol. mp 127-129° C. (chloroform); TLC R_{ƒ}=0.61 (30% ethyl acetate/70% hexanes); IR (CHCl₃) 3489, 2959, 2109 cm⁻¹; ¹H NMR (300 MHz, CDCl₃) 4.09-4.07 (m, 1H), 3.56-3.53 (m, 1H), 3.47 (s, 1H), 2.39 (dd, 1H, J=14.9, 4.3 Hz), 1.97-1.60 (m, 10H), 1.53-1.14 (m, 12H), 1.11 (s, 3H), 0.86 (t, 3H, J=7.3 Hz), 0.56 (s, 3H).

### (3α)-3-Azido-pregn-(5β,6α)-diol (K116)

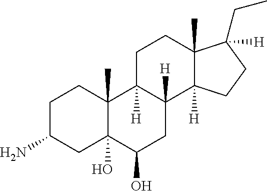

### (3α)-3-Amino-pregn-(5β,6α)-diol (K116)

(3α)-3-Azido-pregn-(5β,6α)-diol (900 mg, 2.59 mmol) was dissolved in 74 mL of diethyl ether. Lithium aluminum hydride (983 mg, 25.9 mmol) was added in one portion. The resulting gray suspension was stirred at room temperature for 24 h. The reaction was then cooled to 0° C. and stirred for 15 minutes. 1 mL of water was slowly added at a rate of one drop every 30 seconds. After stirring the suspension for 5 minutes, 1 mL of 15% NaOH (aq) was then added dropwise at the same rate. Stirring was continued for an additional 5 minutes after which 3 mL of water was slowly added dropwise. The reaction was then stirred until all the salts changed in color from gray to white. Once the salts turned white, the suspension was vacuum-filtered and the salts were thoroughly washed with diethyl ether (3×100 mL). The filtrate was dried with MgSO₄, filtered, and concentrated in vacuo to give amine (3α)-3-amino-pregn-(5β,6α)-diol (696 mg, 80%) as a white solid.

(3α)-3-amino-pregn-(5β,6α)-diol. mp 193-195° C. (chloroform); TLC R_{ƒ}=0.00 (100% ethyl acetate); IR (CHCl₃) 3370, 3294, 2938, 1589 cm⁻¹; ¹H NMR (300 MHz, CDCl₃) □□3.61-3.56 (m, 2H), 2.23 (dd, 1H, J=14.3, 3.7 Hz), 1.99-1.82 (m, 3H), 1.77-1.36 (m, 12H), 1.33-0.99 (m, 10H), 1.23 (s, 3H), 0.87 (t, 3H, J=7.3 Hz), 0.58 (s, 3H).

### (5S,8S,9S,10S,13S,14S)-10,13-dimethyltetradecahydro-1H-cyclopenta[a]phenanthren-3(2H)-one

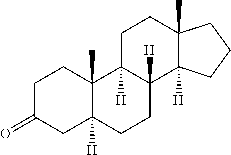

A solution of alcohol 5α-androstan-3β-o1 (1.00 g, 3.62 mmol) in DCM (20 mL) was added to a suspension of pyridinium chlorochromate (1.56 g, 7.24 mmol) and silica gel (1.56 g) in DCM (5 mL). The resulting black orange solution was stirred continuously at rt for 2 h. The reaction mixture was filtered through a plug of silica gel eluting with DCM. The filtrate was concd under reduced pressure which afforded (5S,8S,9S,10S,13S,14S)-10,13-dimethyltetradecahydro-1H-cyclopenta[a]phenanthren-3(2H)-one (0.92 g, 93%).

(5S,8S,9S,10S,13S,14S)-10,13-dimethyltetradecahydro-1H-cyclopenta[a]phenanthren-3(2H)-one. ¹H NMR (300 MHz, CDCl₃) δ 2.18-2.40 (m, 3H), 1.94-2.06 (m, 2H), 1.64-1.71 (m, 2H), 1.57-1.63 (m, 2H), 1.44-1.55 (m, 3H), 1.33-1.43 (m, 2H), 1.24-1.33 (m, 4H), 1.04-1.21 (m, 3H), 0.97 (s, 3H), 0.82-0.95 (m, 2H), 0.73 (dd, J=10.7, 4.2 Hz, 1H), 0.67 (s, 3H); ¹³C NMR (75 MHz, CDCl₃) δ 210.7, 54.0, 53.8, 46.4, 44.3, 40.5, 40.1, 38.5, 38.3, 37.8, 35.4, 35.4, 31.8, 28.7, 25.2, 21.2, 20.2, 17.2.

### (2R,5S,8S,9S,10S,13S,14S)-2-bromo-10,13-dimethyltetradecahydro-1H-cyclopenta[a]phenanthren-3(2H) one

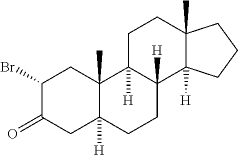

A solution of (5S,8S,9S,10S,13S,14S)-10,13-dimethyltetradecahydro-1H-cyclopenta[a]phenanthren-3(2H)-one (0.18 g, 0.66 mmol) in acetic acid (6.60 mL) was warmed to 50° C. Pyridinium tribromide (0.23 g, 0.66 mmol) was added in one portion and the solution was stirred continuously. After several seconds, a precipitate was formed. The precipitate was filtered, collected, and dried to afford (2R,5S,8S,9S,10S,13S,14S)-2-bromo-10,13-dimethyltetradecahydro-1H-cyclopenta[a]phenanthren-3(2H) one (0.15 g, 65%) as white powdery solid.

(2R,5S,8S,9S,10S,13S,14S)-2-bromo-10,13-dimethyltetradecahydro-1H-cyclopenta[a]phenanthren-3(2H)-one. IR (KBr) 2924, 2865, 2846, 1716, 1656, 1311 cm⁻¹; ¹H NMR (300 MHz, CDCl₃) δ 4.75 (dd, J=13.4, 6.3 Hz, 1H), 2.64 (dd, J=12.6, 6.3 Hz, 1H), 2.37-2.48 (m, 2H), 1.84 (d, J=13.4 Hz, 1H), 1.74-1.77 (m, 1H), 1.69-1.73 (m, 1H), 1.52-1.67 (m, 6H), 1.40-1.47 (m, 2H), 1.32-1.38 (m, 2H), 1.21-1.31 (m, 1H), 1.12-1.19 (m, 2H), 1.08 (s, 3H), 0.88-1.01 (m, 2H), 0.76-0.82 (m, 1H), 0.71 (s, 3H); ¹³C NMR (75 MzZ, CDCl₃) δ 201.5, 54.9, 54.4, 54.1, 52.1, 47.7, 44.2, 41.1, 40.5, 39.4, 38.8, 35.5, 32.1, 28.7, 25.7, 21.8, 20.7, 178, 12.4.

### (2R,5S,8S,9S,10S,13S,14S)-2-azido-10,13-dimethyltetradecahydro-1H-cyclopenta[a]phenanthren-3(2H)-one

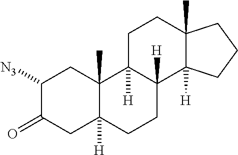

In a flamed dried round bottom flask, (2R,5S,8S,9S,10S,13S,14S)-2-bromo-10,13-dimethyltetradecahydro-1H-cyclopenta[a]phenanthren-3(2H)-one (1.89 g, 5.35 mmol) was suspended in DMF (90 mL). Sodium azide (0.42 g, 6.42 mmol) was added to the suspension and the reaction mixture was stirred continuously at 28° C. After approximately 2 h, the reaction mixture was poured onto crushed ice. After the ice melted it was extracted with ethyl acetate (3×50 mL). The organic layers were collected, combined, washed with cold water (3×50 mL) and brine, dried over magnesium sulfate, and concd under reduced pressure. The residue was recrystallized from ethyl alcohol to afford azide (2R,5S,8S,9S,10S,13S,14S)-2-azido-10,13-dimethyltetradecahydro-1H-cyclopenta[a]phenanthren-3(2H) one (1.35 g, 80%) as light brown solid.

(2R,5S,8S,95,10S,13S,14S)-2-azido-10,13-dimethyltetradecahydro-1H-cyclopenta[a]phenanthren-3(2H)-one m.p.=131.5-132.6° C. (ethyl alcohol); TLC R_{ƒ}=0.63 (hexane: ethyl acetate, 4:1); IR (KBr) 2928, 2833, 2105, 1714, 1282, 1156 cm⁻¹; ¹H NMR (300 MHz, CDCl₃) δ 3.98 (dd, J=13.1, 6.4 Hz, 1H), 2.21-2.42 (m, 3H), 1.69-1.77 (m, 2H), 1.52-1.67 (m, 6H), 1.21-1.48 (m, 6H), 1.13-1.19 (m, 2H), 1.09 (s, 3H), 0.88-1.01 (m, 2H), 0.76-0.86 (m, 1H), 0.71 (s, 3H); ¹³C NMR (75 MHz, CDCl₃) δ 205.5, 64.2, 54.4, 54.2,47.9, 45.8, 44.0, 41.1, 40.5, 38.8, 37.3, 35.4, 32.1, 28.7, 25.7, 21.8, 20.7, 17.7, 12.8.

### 2R-azido-10,13-dimethylhexadecahydro-1H-cyclopenta[a]phenanthren-3R-ol

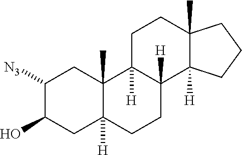

A pre-cooled solution of (2R,5S,8S,9S,10S,13S,14S)-2-azido-10,13-dimethyltetradecahydro-1H-cyclopenta[a]phenanthren-3(2H)-one (1.38 g, 4.37 mmol) in DCM (31 mL) was added to a cooled suspension of sodium borohydride (0.44 g, 11.5 mmol) in DCM: methanol (146 mL, 1:1) at −78° C. using an dry ice/acetone bath with a canula. The reaction mixture was stirred continuously at −78° C. After approximately 2 h, the reaction mixture was quenched by adding sodium hydroxide solution (40 mL, 2 M). The organic layer was collected, dried over sodium sulfate, and concd under reduced pressure. The concentrate was purified through a gravity column chromatography using 10-15-20% ethyl acetate in hexane to afford 2R-azido-10,13-dimethylhexadecahydro-1H-cyclopenta[a]phenanthren-3S-ol (0.19 g, 24%) as white solid and 2R-azido-10,13-dimethylhexadecahydro-1H-cyclopenta[a]phenanthren-3R-ol (0.61 g, 44%) as a clear colorless needle crystals.

2R-azido-10,13-dimethylhexadecahydro-1H-cyclopenta[a]phenanthren-3S-ol. m.p.=76.4-81.3° C. (DCM); TLC R_{ƒ}=0.69 (hexane:ethyl acetate, 4:1); IR (film) 3435, 2927, 2099, 1451, 1248, 1038 cm⁻¹; ¹H NMR (300 MHz, CDCl₃) δ 3.96 (bs, 3H), 3.52 (dq, J=12.5, 4.6, 3.0, Hz, 1H), 2.07 (s, 1H), 1.65-1.80 (m, 3H), 1.53-1.62 (m, 5H), 1.35-1.50 (m, 3H), 1.21-1.32 (m, 3H), 1.08-1.18 (m, 4H), 0.87-1.01 (m, 3H), 0.82 (s, 3H), 0.68 (s, 3H); ¹³C NMR (75 MHz, CDCl₃) δ 68.2, 61.2, 54.6, 54.6, 41.0, 40.6, 38.9, 38.3, 37.2, 37.1, 35.6, 34.5, 32.4, 27.9, 25.7, 21.2, 20.7, 17.8, 12.5.

2R-azido-10,13-dimethylhexadecahydro-1H-cyclopenta[a]phenanthren-3R-ol. m.p.=127.7-130.0° C. (DCM); TLC R_{ƒ}=0.67 (hexane:ethyl acetate, 4:1); IR (film) 3352, 2932, 2862, 2103, 1452, 1262 cm⁻¹, ¹H NMR (300 MHz, CDCl₃) δ 3.32-3.48 (m, 2H), 2.14 (d, J=2.9, Hz, 1H), 2.04 (dd, J=12.8, 4.4 Hz, 1H), 1.67-1.76 (m, 3H), 1.59-1.65 (m, 2H), 1.50-1.58 (m, 2H), 1.38-1.46 (m, 2H), 1.26-1.36 (m, 3H), 1.19-1.23 (m, 1H), 1.11-1.19 (m, 3H), 0.97-1.09 (m, 2H), 0.83-0.93 (m, 2H), 0.87 (s, 3H), 0.73-0.78 (m, 1H), 0.69 (s, 3H); ¹³C NMR (75 MHz, CDCl₃) δ 74.3, 64.8, 54.6, 54.6, 44.6, 42.3, 41.0, 40.6, 38.9, 37.4, 35.9, 35.5, 32.4, 28.2, 25.7, 21.6, 20.7, 17.8, 13.4.

### (2R,3R,5S,8R,9S,10S,13S,14S)-2-azido-10,13-dimethylhexadecahydro-1H-cyclopenta[a]phenanthren-3-yl methanesulfonate

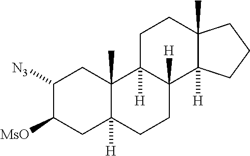

In a round bottom flask, 2R-azido-10,13-dimethylhexadecahydro-1H-cyclopenta[a]phenanthren-3R-ol (0.10 g, 0.31 mmol) was dissolved in pyridine (1.00 mL). Methanesulfonyl chloride (0.04 mL, 0.53 mmol) was added dropwise and the reaction mixture was stirred continuously for 24 h. Water (5 mL) was then added to the reaction mixture to quench the reaction. The quenched reaction mixture was extracted with DCM (3×20 mL). The organic layers were collected, washed with hydrochloric acid (2×10 mL, 6 M) followed by sat. sodium bicarbonate (2×10 mL) and water (2×10 mL). The solution was dried over sodium sulfate and concd under reduced pressure to afford (2R,3R,5S,8R,9S,10S,13S,14S)-2-azido-10,13-dimethylhexadecahydro-1H-cyclopenta[a]phenanthren-3-yl methanesulfonate as an off-white solid (0.11 g, 92%).

(2R,3R,5S,8R,9S,10S,13S,14S)-2-azido-10,13-dimethylhexadecahydro-1H-cyclopenta[a]phenanthren-3-yl methanesulfonate. m.p.=115.0-118.2° C. (DCM); IR (KBr) 2933, 2867, 2105, 1362, 1173, 947 cm⁻¹; ¹H NMR (300 MHz, CDCl₃) δ 4.35 (dt, J=7.8, 5.6 Hz, 1H), 3.57-3.66 (m, 1H), 3.09 (s, 3H), 2.10 (dd, J=13.0, 4.9 1H), 1.94 (ddd, J=13.1, 5.4, 2.3 Hz, 1H), 1.65-1.74 (m, 3H), 1.55-1.64 (m, 3H), 1.41-1.53 (m, 2H), 1.32-1.38 (m, 2H), 1.18-1.30 (m, 4H), 1.05-1.15 (m, 4H), 0.89-1.01 (m, 1H), 0.87 (s, 3H), 0.75 (dd, J=12.0, 4.1 Hz, 1H), 0.68 (s, 3H); ¹³C NMR (75 MHz, CDCl₃) δ 84.1, 60.7, 54.2, 54.1, 44.2, 42.8, 40.3, 38.6, 35.2, 34.7, 32.0, 27.4, 25.5, 21.3, 20.5, 17.5, 12.9.

### (2R,3S,5S,8R,9S,10S,13S,14S)-2,3-diazido-10,13-dimethylhexadecahydro-1H-cyclopenta[a]phenanthrene

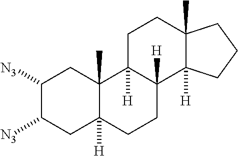

A suspension of (2R,3R,5S,8R,9S,10S,13S,14S)-2-azido-10,13-dimethylhexadecahydro-1H-cyclopenta[a]phenanthren-3-yl methanesulfonate (0.32 g, 0.30 mmol) and sodium azide (0.04 g, 0.60 mmol) in DMF (3 mL) was heated to 100° C. After 20 h, the reaction mixture was cooled and was added to crushed ice. The quenched reaction mixture was extracted with ethyl acetate (3×10 mL) and the organic layers were collected, combined, washed with water (5×10 mL), dried over sodium sulfate and concd under reduced pressure to give (2R,3S,5S,8R,9S,10S,13S,14S)-2,3-diazido-10,13-dimethylhexadecahydro-1H-cyclopenta[a]phenanthrene (0.09 g, 90%) as an off-white solid.

(2R,3S,5S,8R,9S,10S,13S,14S)-2,3-diazido-10,13-dimethylhexadecahydro-1H-cyclopenta[a]phenanthrene. m.p.=247.5° C. (ethyl acetate) (dec.); IR (KBr) 2930, 2861, 2100, 1452, 1363, 1263 cm⁻¹; ¹H NMR (300 MHz, CDCl₃) δ 3.91 (q, J=2.7 Hz, 1H), 3.49 (td, J=12.4, 3.9 Hz, 1H), 1.80 (dd, J=12.5, 3.9 Hz, 1H), 1.67-1.76 (m, 2H), 1.62-1.66 (m, 1H), 1.57-1.61 (m, 2H), 1.50-1.56 (m, 2H), 1.45-1.47 (m, 1H), 1.39-1.43 (m, 2H), 1.29-1.37 (m, 2H), 1.21-1.27 (m, 2H), 1.14-1.18 (m, 2H), 1.07-1.12 (m, 2H), 0.96-1.03 (m, 1H), 0.86-0.94 (m, 2H), 0.82 (s, 3H), 0.69 (s, 3H); ¹³C NMR (75 MHz, CDCl₃) δ 61.9, 58.9, 54.5, 54.4, 41.0, 40.5, 39.3, 38.8, 37.6, 37.1, 35.4, 32.7, 32.2, 27.7, 25.6, 21.1, 20.6, 17.7, 12.8.

### (2R,3S,5S,8R,9S,10S,13S,14S)-10,13-dimethylhexadecahydro-1H-cyclopenta[a]phenanthrene-2,3-diaminium chloride (K161)

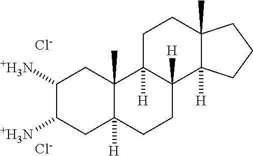

In a round bottom flask, LAH (0.16 g, 4.03 mmol) was suspended in THF (2 mL). The suspension was cooled at 0° C. using an ice/water bath. A solution of (2R,3S,5S,8R,9S,10S,13S,14S)-2,3-diazido-10,13-dimethylhexadecahydro-1H-cyclopenta[a]phenanthrene (0.21 g, 0.61 mmol) in THF (3 mL) was added. The reaction mixture was warmed to rt and then heated to reflux for 4 h. It was then cooled to rt and diluted with THE (5 mL). The diluted reaction mixture was cooled at 0° C. using ice/water bath and was quenched using the Fieser method.⁸⁵ The quenched reaction mixture was filtered through celite, dried over sodium sulfate, and concd under reduced pressure. The residue was dissolved in diethyl ether:CHCl₃ (20 mL, 1:1). The solution was heated and filtered to remove any undissolved amine. A solution of HCl in diethyl ether (1.04 mmol, 0.52 mL, 2.0 M in diethyl ether) was then added dropwise which resulted to formation of white precipitate. The precipitate was filtered, washed with diethyl ether, and dried to afford (2R,3S,5S,8R,9S,10S,13S,14S)-10,13-dimethylhexadecahydro-1H-cyclopenta[a]phenanthrene-2,3-diaminium chloride (0.17 g, 77% over 2 steps).

(2R,3S,5S,8R,9S,10S,13S,14S)-10,13-dimethylhexadecahydro-1H-cyclopenta[a]phenanthrene-2,3-diaminium chloride. m.p.=261.4° C. (diethyl ether) (dec.); IR (KBr) 3430, 2931, 2861, 1602, 1094, 1030 cm⁻¹; ¹H NMR (300 MHz, CD₃OD) δ 3.85 (app d, J=2.9 Hz, 1H), 3.70 (td, J=13.4, 3.7 Hz, 1H), 1.87-1.99 (m, 2H), 1.70-1.80 (m, 3H), 1.65-1.68 (m, 2H), 1.55-1.62 (m, 3H), 1.40-1.50 (m, 3H), 1.30-1.37 (m, 3H), 1.11-1.27 (m, 4H), 0.98-1.09 (m, 2H), 0.95 (s, 3H), 0.74 (s, 3H); ¹³C NMR (75 MHz, CD₃OD) δ 55.7, 54.9, 50.8, 49.7, 42.1, 41.5, 39.9, 39.6, 38.5, 37.6, 36.7, 33.1, 32.1, 28. 5, 26 6, 22 2, 21.5, 18.0, 12.8.

The foregoing description is intended to illustrate but not to limit the scope of the disclosure, which is defined by the scope of the appended claims. Other embodiments are within the scope of the following claims.

1. A method of activating microglial cells in a subject in need thereof comprising:
administering an effective amount of a SHIP inhibitor to the subject, wherein the SHIP inhibitor is a pan-SHIP1/2 inhibitor or a SHIP2 inhibitor.

2. The method of claim 1, wherein the SHIP inhibitor is the pan-SHIP1/2 inhibitor.

3. The method of claim 1, wherein the SHIP inhibitor is the SHIP2 inhibitor.

4. The method of claim 1, wherein the subject has an illness or condition for which microglial activation is therapeutic.

5. The method of claim 4, wherein the illness or condition is a neurodegenerative disorder.

6. The method of claim 5, wherein the neurodegenerative disorder is characterized by death of neurons.

7. The method of claim 5, wherein the neurodegenerative disorder is characterized by misfolded proteins.

8. The method of claim 7, wherein the misfolded proteins are beta-amyloid, comprising the beta-amyloid (1-42) peptide.

9. The method of claim 4, wherein the illness or condition is Alzheimer's disease.

10. The method of any of claims 1-9, wherein activating microglial cells results in one or more of the following:
promotion of the proliferation of microglial cells;
an increase in the size of the lysosomal/phagosomal compartment in microglial cells;
an increase in the phagocytosis of dead neurons by microglial cells; or
an increase in the phagocytosis of beta-amyloid protein or beta-amyloid (1-42) peptide by microglial cells.

11. The method of claim 10, wherein activating microglial cells results in an increase in the size of the lysosomal/phagosomal compartment in microglial cells.

12. The method of claim 10, wherein activating microglial cells results in an increase in the phagocytosis by of dead neurons by microglial cells.

13. The method of claim 10, wherein activating microglial cells results in an increase in the phagocytosis of beta-amyloid protein or beta-amyloid (1-42) peptide by microglial cells.

14. The method of claim 10, wherein the microglial cells showing an increase in the phagocytosis of dead neurons are CD11b+CD45 low cells.

15. The method of claim 10, wherein the microglial cells showing an increase in the phagocytosis of beta amyloid protein or beta-amyloid peptide are CD11b+CD45 low cells.

16. The method of claim 1, wherein the SHIP inhibitor is a compound of Formula (I): 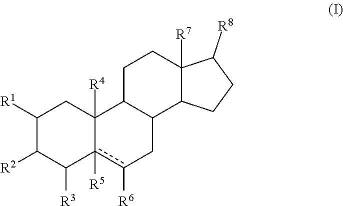
wherein [symbol]
represents a single or double bond;
R¹ is selected from the group consisting of hydrogen, hydroxy, mercapto, C₁-C₆ alkoxy, aryloxy, C₁-C₆ alkylthio, C₆-C₁₂ arylthio, C₁-C₆ alkylcarbonamido, C₆-C₁₂ arylcarbonamido, C₁-C₆ alkylsulfonamido, C₆-C₁₂ arylsulfonamido, substituted or unsubstituted amino, oxycarbonyl and C₁-C₆ aminoalkyl;
R² is selected from the group consisting of hydrogen, hydroxy, mercapto, C₁-C₆ alkoxy, aryloxy, C₁-C₆ alkylthio, C₆-C₁₂ arylthio, C₁-C₆ alkylcarbonamido, C₆-C₁₂ arylcarbonamido, C₁-C₆ alkylsulfonamido, C₆-C₁₂ arylsulfonamido, substituted or unsubstituted amino, oxycarbonyl and C₁-C₆ aminoalkyl; R³ is hydrogen, C₁-C₄ alkyl, or C₁-C₄ haloalkyl;
R³ is hydrogen, C₁-C₄ alkyl, or C₁-C₄ haloalkyl;
R⁴ is hydrogen, C₁-C₄ alkyl, or C₁-C₄ haloalkyl;
R⁵ is hydrogen, C₁-C₄ alkyl, hydroxy, C₁-C₆ alkoxy, mercapto, C₁-C₆ alkylthio, substituted or unsubstituted amino, or C₁-C₄ haloalkyl;
R⁶ is hydrogen, C₁-C₄ alkyl, hydroxy, C₁-C₆ alkoxy, mercapto, C₁-C₆ alkylthio, substituted or unsubstituted amino, or C₁-C₄ haloalkyl;
R⁷ is hydrogen, C₁-C₄ alkyl, or C₁-C₄ haloalkyl; and
R⁸ is hydrogen, C₁-C₁₂ alkyl, or C₁-C₁₂ haloalkyl, and pharmaceutically acceptable esters, salts, and prodrugs thereof.

17. The method of claim 16, wherein R¹ and R² are a substituted or unsubstituted amino.

18. The method of claim 16, wherein R¹ and R² are NH₂ or NH₃Cl.

19. The method of claim 16, wherein the compound of Formula (I) is selected from the group consisting of: 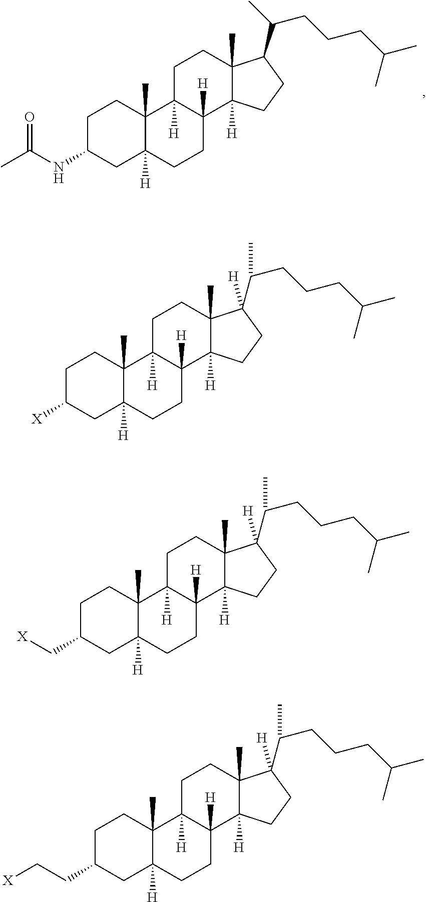 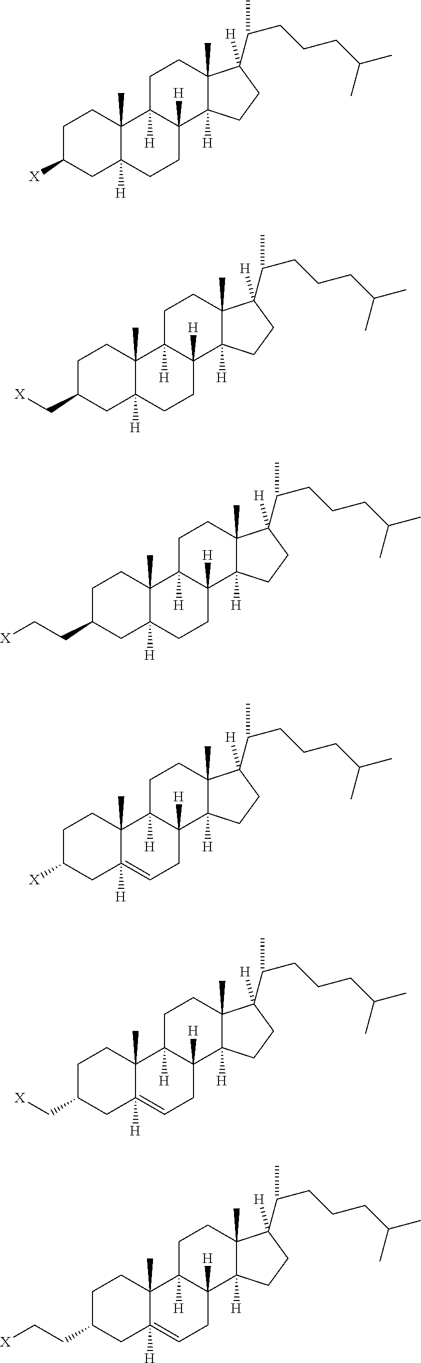 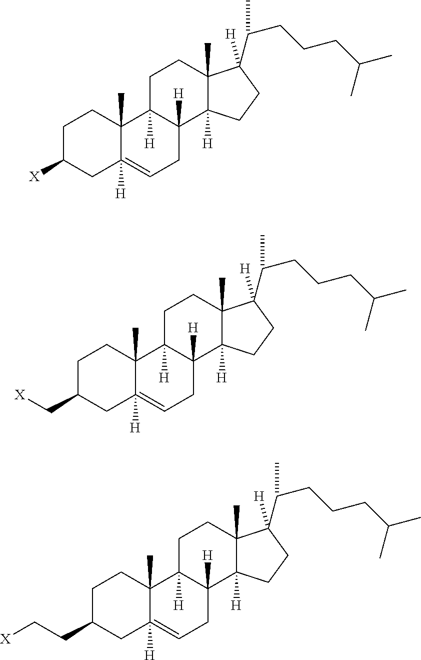 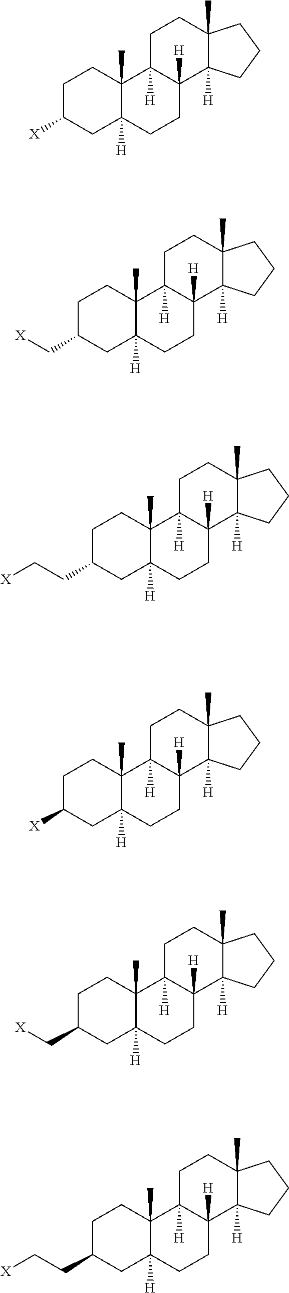 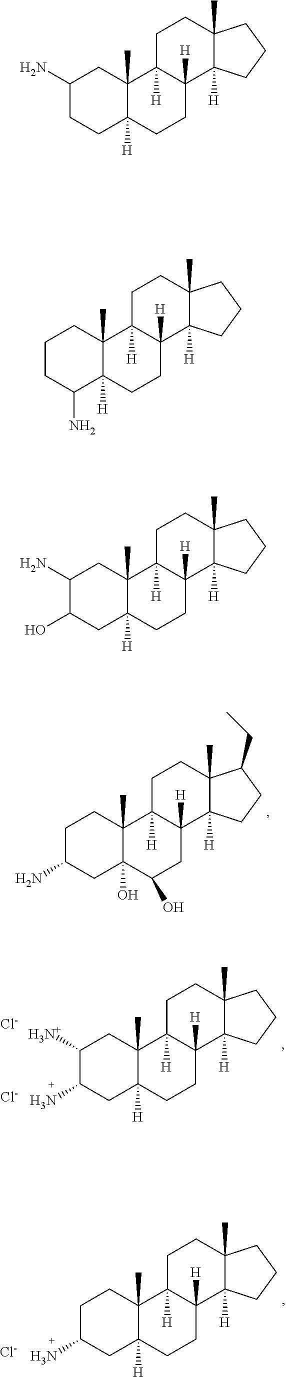 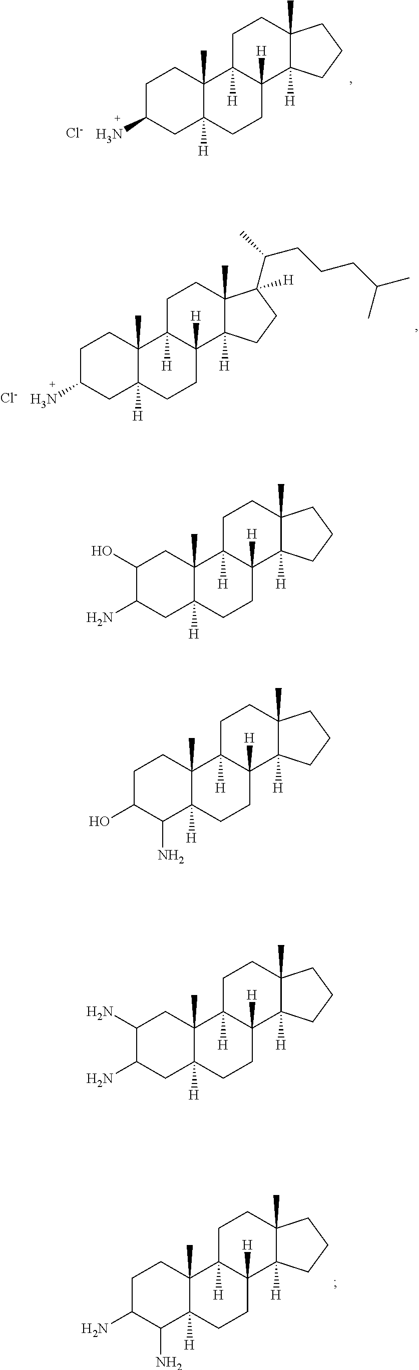
and
pharmaceutically acceptable salts thereof, wherein X=NR₂, NRCOR, NHCONR₂, OR, SR, OCOR, OCONR₂, or NHCNHNH₂, and wherein R=H, alkyl, cycloalkyl, aryl, or benzyl

20. The method of claim 16, wherein the compound of Formula (I) is a compound of Formula (II): 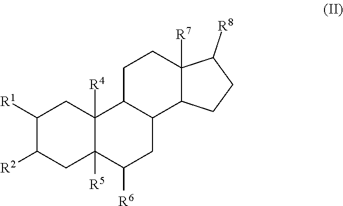
wherein
R¹ is selected from the group consisting of hydrogen, hydroxy, substituted or unsubstituted amino, and C₁-C₆ aminoalkyl;
R² is selected from the group consisting of hydrogen, hydroxy, substituted or unsubstituted amino, and C₁-C₆ aminoalkyl;
R⁴ is C₁-C₄ alkyl;
R⁵ is hydrogen, C₁-C₄ alkyl, or hydroxy;
R⁶ is hydrogen, C₁-C₄ alkyl, or hydroxy;
R⁷ is C₁-C₄ alkyl; and
R⁸ is hydrogen or C₁-C₁₂ alkyl, and pharmaceutically acceptable esters, salts, and prodrugs thereof.

21. The method of claim 20, wherein the substituted or unsubstituted amino is NH₂ or NH₃Cl.

22. The method of claim 20, wherein R¹ and R² are a substituted or unsubstituted amino.

23. The method of claim 20, wherein R¹ and R² are NH₂ or NH₃Cl.

24. The method of claim 20, wherein R⁵ and R⁶ are hydroxy.

25. The method of claim 20, wherein R⁸ is C₁-C₁₂ alkyl.

26. The method of claim 20, wherein R⁸ is hydrogen.

27. The method of claim 20 comprising administering to a subject a hydrochloride salt of the compound of Formula (II).

28. The method of claim 20, wherein the compound of Formula (II) is selected from the group consisting of:

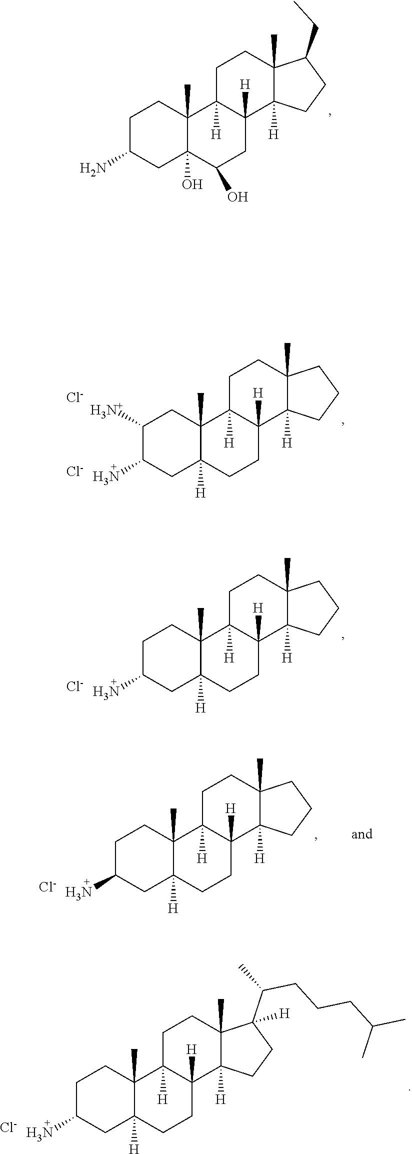

29. The method of claim 1, wherein the SHIP inhibitor is a compound of Formula (III): 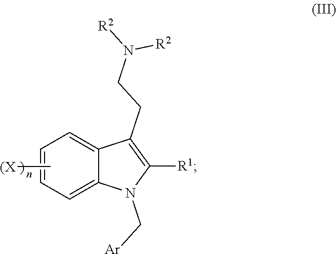
wherein,
Ar is a C₅₋₆ aryl;
R¹ is selected from the group consisting of H, C₁₋₄ alkyl, and C₅₋₆ aryl;
each R² is independently selected from the group consisting of H, C₁₋₄ alkyl, and C₅₋₆ aryl;
each X, if present, is independently selected from the group consisting of halo, C₁₋₄ alkyl, C₅₋₆ aryl, and —Y—R³;
wherein Y is selected from the group consisting of —S—, —NH—, —O—; and
R³ is H or C₁₋₄ alkyl; and
n is 0-4, and pharmaceutically acceptable esters, salts, and prodrugs thereof.

30. The method of claim 29, wherein the compound of Formula (III) is selected from the group of 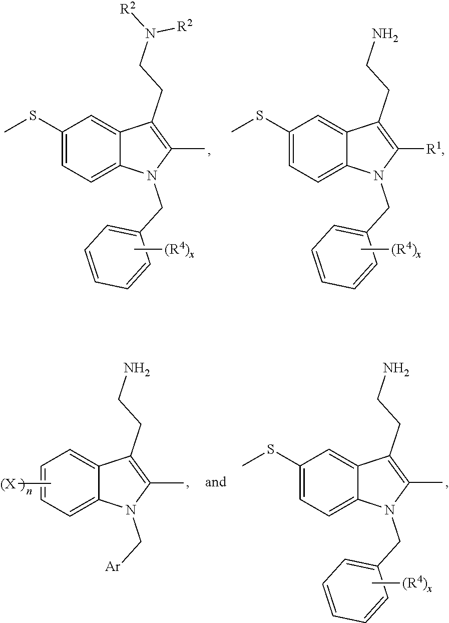
where R⁴ is halo, and x is 0-5, and pharmaceutically acceptable esters, salts, and prodrugs thereof.

31. The method of claim 29, wherein the compound of Formula (III) is selected from the group consisting of: 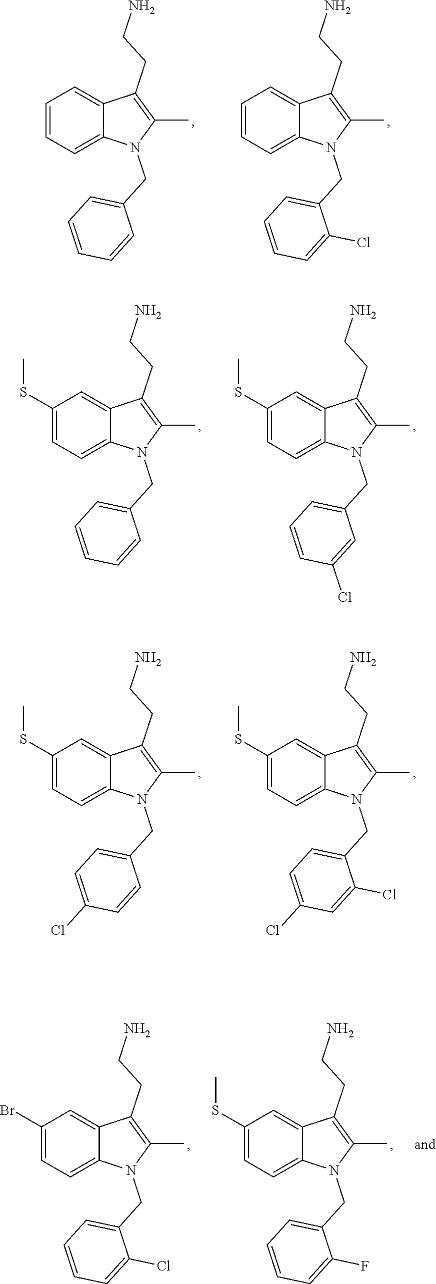 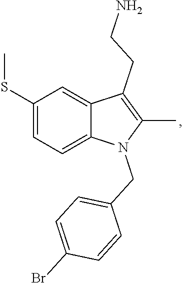 [SMILES: CSc1ccc2c(c1)c(CCN)c(C)n2Cc1ccc(Br)cc1]
and
pharmaceutically acceptable esters, salts, and prodrugs thereof.

32. A method of improving one or more symptoms of a neurodegenerative disorder in a subject in need thereof comprising:
administering a therapeutically effective amount of a SHIP inhibitor to the subject, wherein the SHIP inhibitor is a pan-SHIP1/2 inhibitor or a SHIP2 inhibitor, wherein said inhibitor activates microglial cells.

33. The method of claim 32, wherein the SHIP inhibitor is the pan-SHIP1/2 inhibitor.

34. The method of claim 32, wherein the SHIP inhibitor is the SHIP2 inhibitor.

35. The method of claim 32, wherein the neurodegenerative disorder is characterized by death of neurons.

36. The method of claim 32, wherein the neurodegenerative disorder is characterized by misfolded proteins.

37. The method of claim 36, wherein the misfolded proteins are beta-amyloid, comprising the beta-amyloid (1-42) peptide.

38. The method of claim 32, wherein the neurodegenerative disorder is Alzheimer's disease.

39. The method of any of claims 32-38, wherein activating microglial cells results in one or more of the following:
promotion of the proliferation of microglial cells;
an increase in the size of the lysosomal/phagosomal compartment in microglial cells;
an increase in the phagocytosis of dead neurons by microglial cells; and
an increase in the phagocytosis of beta-amyloid protein or beta-amyloid (1-42) peptide by microglial cells.

40. The method of claim 39, wherein activating microglial cells results in an increase in the size of the lysosomal/phagosomal compartment in microglial cells.

41. The method of claim 39, wherein activating microglial cells results in an increase in the phagocytosis of dead neurons.

42. The method of claim 39, wherein activating microglial cells results in an increase in the phagocytosis of beta-amyloid protein or beta-amyloid (1-42) peptide.

43. The method of claim 39, wherein the microglial cells showing an increase in the phagocytosis of dead neurons are CD11b+CD45 low cells.

44. The method of claim 39, wherein the microglial cells showing an increase in the phagocytosis of beta amyloid protein or beta-amyloid peptide are CD11b+CD45 low cells.

45. The method according to any of claims 32-44, wherein the pan-SHIP1/2 inhibitor is administered to a subject in a continuous manner or in a pulsatile manner.

46. The method according to any of claims 32-44, wherein the pan-SHIP1/2 inhibitor is administered to a subject at least daily for a period of at least 2, 3, 4, 5, 6, 7, 8, 9, 10, 12, 14, 16, 18, 20 or more days.

47. The method according to any of claims 32-44, wherein the pan-SHIP1/2 inhibitor is administered peripherally to the subject.

48. The method of claim 32, wherein the SHIP inhibitor is a compound of formula (I): 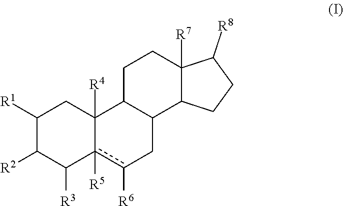
wherein [symbol]
represents a single or double bond;
R¹ is selected from the group consisting of hydrogen, hydroxy, mercapto, C₁-C₆ alkoxy, aryloxy, C₁-C₆ alkylthio, C₆-C₁₂ arylthio, C₁-C₆ alkylcarbonamido, C₆-C₁₂ arylcarbonamido, C₁-C₆ alkylsulfonamido, C₆-C₁₂ arylsulfonamido, substituted or unsubstituted amino, oxycarbonyl and C₁-C₆ aminoalkyl;
R² is selected from the group consisting of hydrogen, hydroxy, mercapto, C₁-C₆ alkoxy, aryloxy, C₁-C₆ alkylthio, C₆-C₁₂ arylthio, C₁-C₆ alkylcarbonamido, C₆-C₁₂ arylcarbonamido, C₁-C₆ alkylsulfonamido, C₆-C₁₂ arylsulfonamido, substituted or unsubstituted amino, oxycarbonyl and C₁-C₆ aminoalkyl; R³ is hydrogen, C₁-C₄ alkyl, or C₁-C₄ haloalkyl;
R³ is hydrogen, C₁-C₄ alkyl, or C₁-C₄ haloalkyl;
R⁴ is hydrogen, C₁-C₄ alkyl, or C₁-C₄ haloalkyl;
R⁵ is hydrogen, C₁-C₄ alkyl, hydroxy, C₁-C₆ alkoxy, mercapto, C₁-C₆ alkylthio, substituted or unsubstituted amino, or C₁-C₄ haloalkyl;
R⁶ is hydrogen, C₁-C₄ alkyl, hydroxy, C₁-C₆ alkoxy, mercapto, C₁-C₆ alkylthio, substituted or unsubstituted amino, or C₁-C₄ haloalkyl;
R⁷ is hydrogen, C₁-C₄ alkyl, or C₁-C₄ haloalkyl; and
R⁸ is hydrogen, C₁-C₁₂ alkyl, or C₁-C₁₂ haloalkyl, and pharmaceutically acceptable esters, salts, and prodrugs thereof.

49. The method of claim 48, wherein R¹ and R² are a substituted or unsubstituted amino.

50. The method of claim 48, wherein R¹ and R² are NH₂ or NH₃Cl.

51. The method of claim 48, having the formula of: 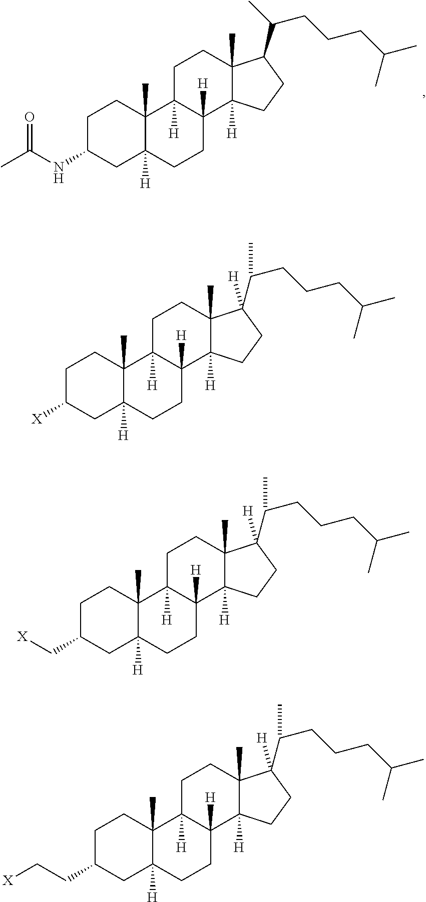 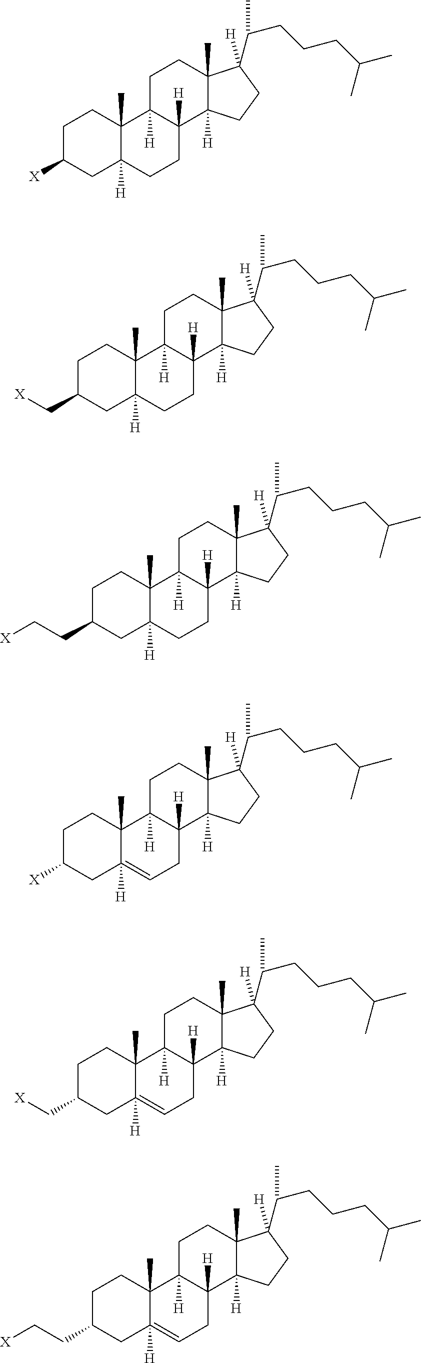 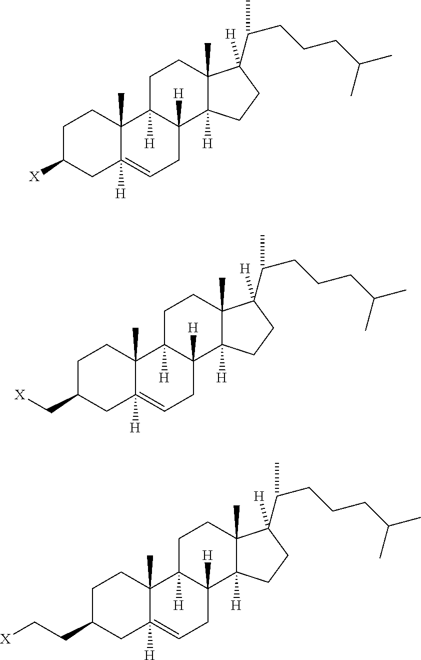 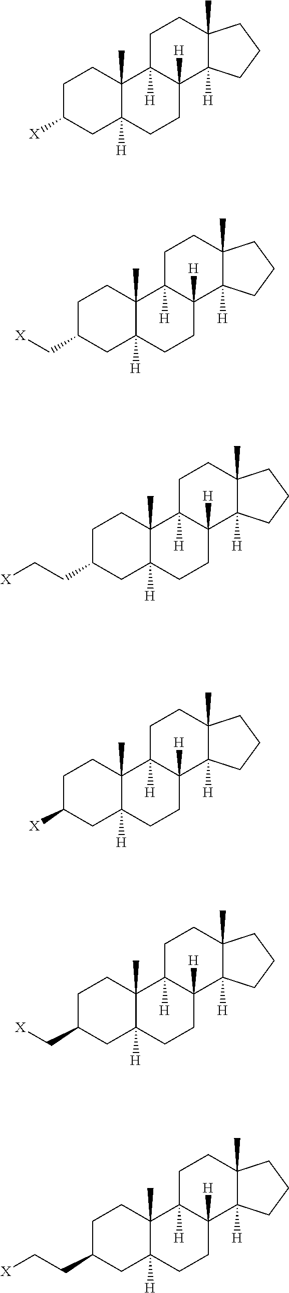 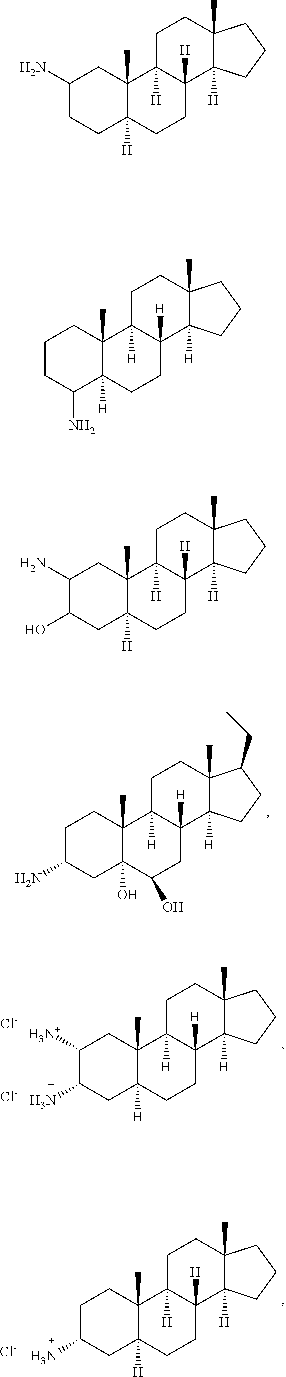 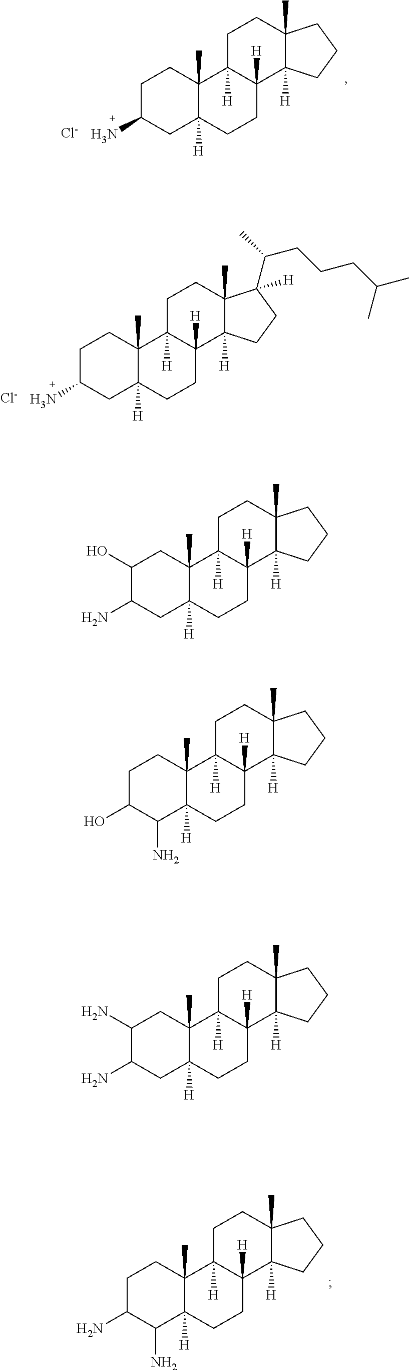
and
pharmaceutically acceptable salts thereof, wherein X=NR₂, NRCOR, NHCONR₂, OR, SR, OCOR, OCONR₂, or NHCNHNH₂, and wherein R=H, alkyl, cycloalkyl, aryl, or benzyl.

52. The method of claim 48, wherein the compound of Formula (I) is a compound of Formula (II): 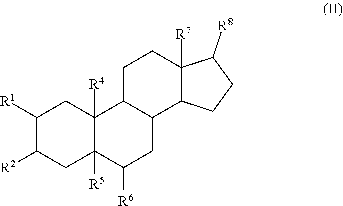
wherein
R¹ is selected from the group consisting of hydrogen, hydroxy, substituted or unsubstituted amino, and C₁-C₆ aminoalkyl;
R² is selected from the group consisting of hydrogen, hydroxy, substituted or unsubstituted amino, and C₁-C₆ aminoalkyl;
R⁴ is C₁-C₄ alkyl;
R⁵ is hydrogen, C₁-C₄ alkyl, or hydroxy;
R⁶ is hydrogen, C₁-C₄ alkyl, or hydroxy;
R⁷ is C₁-C₄ alkyl; and
R⁸ is hydrogen or C₁-C₁₂ alkyl, and pharmaceutically acceptable esters, salts, and prodrugs thereof.

53. The method of claim 52, wherein the substituted or unsubstituted amino is NH₂ or NH₃Cl.

54. The method of claim 52, wherein R¹ and R² are a substituted or unsubstituted amino.

55. The method of claim 52, wherein R¹ and R² are NH₂ or NH₃Cl.

56. The method of claim 52, wherein R⁵ and R⁶ are hydroxy.

57. The method of claim 52, wherein R⁸ is C₁-C₁₂ alkyl.

58. The method of claim 52, wherein R⁸ is hydrogen.

59. The method of claim 52 comprising administering to a subject a hydrochloride salt of the compound of Formula (II).

60. The method of claim 52, wherein the compound of Formula (II) is selected from the group consisting of:

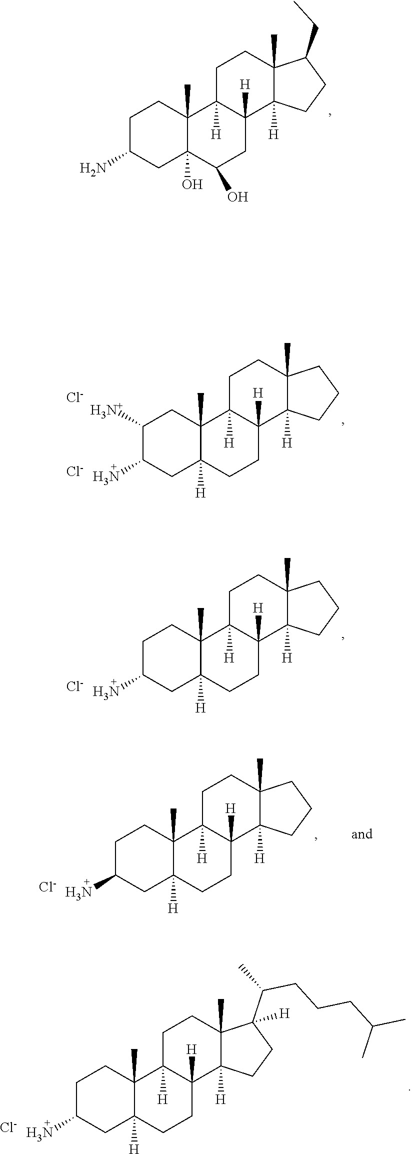

61. The method of claim 32, wherein the SHIP inhibitor is a compound of Formula (III): 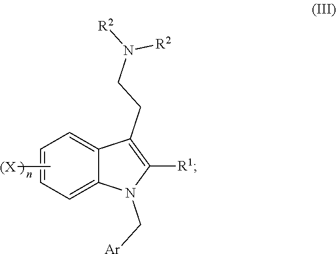
wherein,
Ar is a C₅₋₆ aryl;
R¹ is selected from the group consisting of H, C₁₋₄ alkyl, and C₅₋₆ aryl;
each R² is independently selected from the group consisting of H, C₁₋₄ alkyl, and C₅₋₆ aryl;
each X, if present, is independently selected from the group consisting of halo, C₁₋₄ alkyl, C₅₋₆ aryl, and —Y—R³;
wherein Y is selected from the group consisting of —S—, —NH—, —O—; and
R³ is H or C₁₋₄ alkyl; and
n is 0-4, and pharmaceutically acceptable esters, salts, and prodrugs thereof.

62. The method of claim 61, wherein the compound of Formula (III) is selected from the group consisting of: 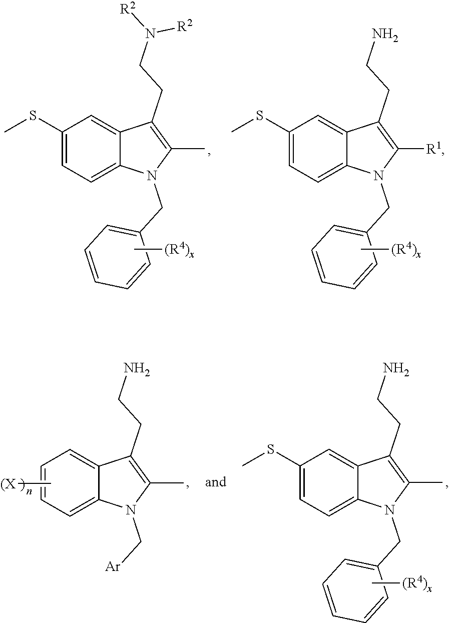
where R⁴ is halo, and×is 0-5, and pharmaceutically acceptable esters, salts, and prodrugs thereof.

63. The method of claim 61, wherein the compound of Formula (III) is selected from the group consisting of: 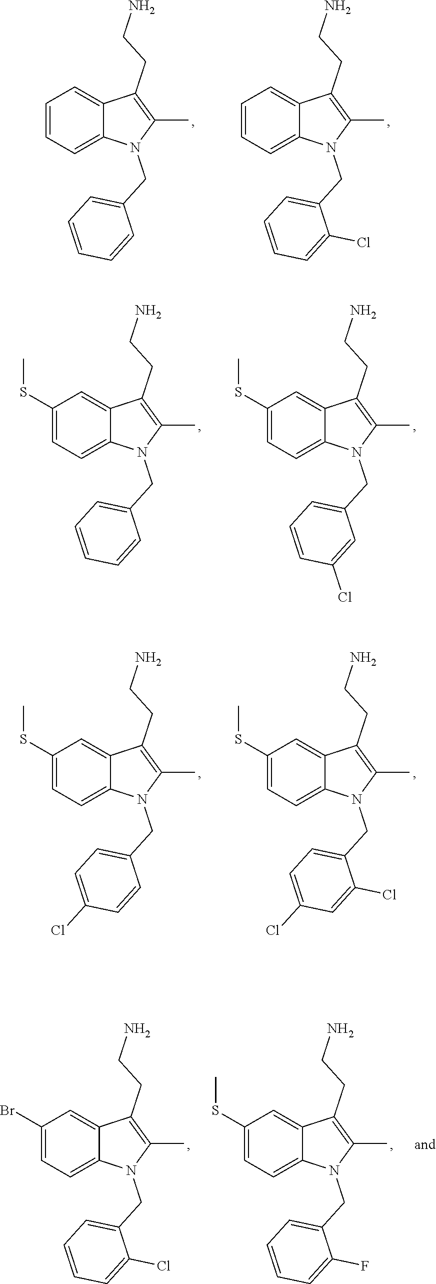 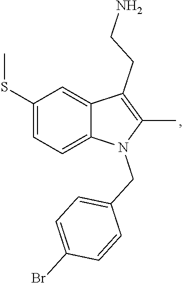 [SMILES: CSc1ccc2c(c1)c(CCN)c(C)n2Cc1ccc(Br)cc1]
and pharmaceutically acceptable esters, salts, and prodrugs thereof.
